SEQUENCE LISTING (2 sequences)

SEQ ID NO: 1 - protein, 117 aa, Homo sapiens
MNAPPAFESFLLFEGEKKITINKDTKVPNACLFTINKEDHTLGNIIKSQLLKDPQVLFAGYKVPHPLEHKIIIRVQTTPDYSPQEAFTNAITDLISELSLLEERFRVAIKDKQEGIE

SEQ ID NO: 2 - protein, 136 aa, Homo sapiens
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLARRIRGERA

(19) United States
(12) Patent Application Publication
(10) Pub. No.: US 2018/0371550 A1
(43) Pub. Date: Dec. 27, 2018

(54) LOSS OF TRANSCRIPTIONAL FIDELITY LEADS TO IMMUNOTHERAPY RESISTANCE IN CANCERS

(71) Applicant: Children's Hospital Medical Center, Cincinnati, OH (US)

(72) Inventor: Kakajan Komurov, West Chester, OH (US)

(21) Appl. No.: 15/742,475

(22) PCT Filed: Jul. 8, 2016
(86) PCT No.: PCT/US16/41607

§ 371 Date: Jan. 5, 2018

Related U.S. Application Data
(60) Provisional application No. 62/189,935, filed on Jul. 8, 2015.

(51) Int. Cl. C12Q 1/6886

(52) U.S. Cl. CPC C12Q 1/6886; C12Q 2600/158; C12Q 2600/106

(57) ABSTRACT

Methods and compositions disclosed herein generally relate to determining suitability of immunotherapy for a subject having cancer, by determining whether tumor cells from a subject having cancer or one or more symptoms thereof have a loss of transcriptional fidelity (LTF) phenotype. Embodiments of the invention relate to methods of stratifying one or more subjects in a clinical trial by determining whether tumor cells from one or more subjects having cancer or one or more symptoms thereof have an LTF phenotype. Embodiments of the invention also relate to diagnostic kits, tests, or arrays to test for presence of a loss of transcriptional fidelity (LTF) phenotype in a sample.

### CROSS REFERENCE TO RELATED APPLICATION

The present application claims the benefit of priority under 35 U.S.C. § 119(e) to U.S. Provisional Application No. 62/189,935, GLOBAL CRYPTIC TRANSCRIPTION DEFINES A NOVEL SUBCLASS IN HUMAN CANCERS, filed on Jul. 8, 2105, which is currently co-pending herewith and which is incorporated by reference in its entirety.

### FIELD OF THE INVENTION

Particular aspects of the invention disclosed herein generally relate to determination of the presence of a loss of transcriptional fidelity (LTF) phenotype in a subject, and in more particular aspects, to cancer treatment based on the determination of an LTF phenotype in a subject having cancer.

### BACKGROUND

Gene expression is a complex process that involves dynamic interplay of epigenetic and core transcriptional machineries. Proper histone modification and remodeling dynamics are essential for the positioning and kinetics of RNA Polymerase II (RNAP II) transcription along the gene, as well as for the recruitment and function of the mRNA processing machinery (Luco et al., 2010; Venkatesh and Workman, 2015). Deregulation of the histone or RNAP II post-transcriptional modifications can severely compromise transcriptional fidelity and lead to the production of spurious transcripts (Venkatesh and Workman, 2015).

Cancer pathogenicity partly relies on deregulated gene expression processes, and deregulation of mRNA transcription is a hallmark of many cancers. As such, many of the most frequently genetically altered genes in cancers, such as TP53 and MYC, encode sequence-specific transcription factors. Recently, somatic mutations in a number of generic transcriptional regulators, such as chromatin remodelers (e.g. SETD2, EP300, MLL3) and core mRNA transcription and splicing complexes (e.g. POLR2A, MED12, SF3B1, U2AF1), have also been identified (Plass et al., 2013; Watson et al., 2013).

### SUMMARY OF THE INVENTION

Embodiments of the invention encompass methods for determining suitability of immunotherapy for a subject having cancer, wherein the methods include: analyzing, by RNA analysis, a sample having tumor cells from a subject having cancer to determine whether the tumor cells have a loss of transcriptional fidelity (LTF) phenotype characterized by having a preferential expression or higher proportion of one or more aberrant or non-canonical mRNA isoforms, relative to a control value; and determining a lack of suitability of immunotherapy where the tumor cells of the subject have an LTF phenotype, or determining a suitability of immunotherapy where the tumor cells of the subject lack an LTF phenotype. In some embodiments, the LTF phenotype further includes reduced expression or reduced presence of one or more proteins selected from RNAP II Ser2, RNAP II Ser5, H3K4me3, H3K27me3, and H3K36me3.

Embodiments of the invention also encompass methods of determining suitability of immunotherapy for a subject having cancer, including: analyzing, by protein analysis, a sample having tumor cells from a subject having cancer to determine whether the tumor cells have a loss of transcriptional fidelity (LTF) phenotype characterized by reduced expression or reduced presence of one or more proteins selected from RNAP II Ser2, RNAP II Ser5, H3K4me3, H3K27me3, and H3K36me3 relative to a respective control value; and determining a lack of suitability of immunotherapy where the tumor cells of the subject have an LTF phenotype, or determining a suitability of immunotherapy where the tumor cells of the subject lack an LTF phenotype. In some embodiments, the LTF phenotype further includes a preferential expression or higher proportion, relative to that of normal cells, to that of non-LTF tumor cells, or to that of mRNA corresponding to one or more internal control genes of the tumor cells not affected by LTF, of one or more aberrant or non-canonical mRNA isoform(s) of corresponding normal or canonical mRNA isoform(s), including full-length isoforms.

In some embodiments of the methods, the control value can be that of normal cells, that of non-LTF tumor cells, or that of mRNA corresponding to one or more internal control genes of the tumor cells not affected by LTF. In some embodiments, the one or more internal control genes of the tumor cells not affected by LTF, include one or more type II genes as defined herein.

In some embodiments, the one or more aberrant or non-canonical mRNA isoform(s) include aberrant or non-canonical mRNA isoform(s) lacking exon and/or intron sequences found in the corresponding normal or canonical mRNA isoform(s), including full-length isoforms, or retaining exon and/or intron sequences not found in the corresponding normal or canonical mRNA isoform(s), including full-length isoforms. In some embodiments, the one or more aberrant or non-canonical mRNA isoform(s) include aberrant or non-canonical mRNA isoform(s) lacking 5′-exon sequences found in the corresponding normal or canonical mRNA isoform(s), including full-length isoforms, or retaining 5′exon sequences not found in the corresponding normal or canonical mRNA isoform(s), including full-length isoforms. In some embodiments, the one or more aberrant or non-canonical mRNA isoform(s) include aberrant or non-canonical mRNA isoform(s) having an increased amount of retained intron-exon junctions compared to the corresponding normal or canonical mRNA isoform(s), including full-length isoforms. In some embodiments, the one or more aberrant or non-canonical mRNA isoform(s) include an aberrant or non-canonical mRNA lacking exon sequences required for encoding a protein encoded by a corresponding normal or canonical mRNA isoform including full-length mRNA isoforms thereof.

In some embodiments, the aberrant or non-canonical mRNA isoform(s) encode one or more protein(s) that can be shorter than the corresponding full-length protein by less than 98%, less than 97%, less than 95%, less than 90%, less than 85%, less than 80%, less than 75%, less than 70%, and less than 60%. In some embodiments, for a given mRNA, greater than 50%, greater than 60%, greater than 70%, greater than 80%, greater than 90%, or greater than 95% of the mRNA can be present as corresponding aberrant or non-canonical mRNA isoforms. In some embodiments, for a given mRNA, greater than 50%, greater than 60%, greater than 70%, greater than 80%, greater than 90%, or greater than 95% of the mRNA expression can be of the corresponding aberrant or non-canonical mRNA isoform. In some embodiments, the one or more aberrant or non-canonical mRNA isoforms can be aberrant or non-canonical mRNA isoforms of corresponding normal or canonical mRNAs, including full-length mRNAs, having lengths of greater than 10 kb, greater than 25 kb, greater than 40 kb, greater than 50 kb, greater than 75 kb, greater than 100 kb, greater than 150 kb, or greater than 200 kb.

In some embodiments, the one or more aberrant or non-canonical mRNA isoforms can be encoded by one or more corresponding genes involved in RNA polymerase II (RNAP II) transcription and/or processing and/or in histone H3 modification and/or chromatin remodeling. In some embodiments, the RNAP II genes include genes involved in RNAP II phosphorylation and/or wherein the genes involved in histone H3 modification and/or chromatin remodeling include genes in involved in histone H3 methylation and/or acetylation. In some embodiments, the genes involved in RNAP II phosphorylation include genes involved in RNAP II phosphorylation at amino acid positions Ser2 and/or Ser5. In some embodiments, the genes involved in histone H3 methylation include genes involved in histone H3 methylation at amino acid positions K4, K27, and/or K36. In some embodiments, the one or more genes involved in RNA polymerase II (RNAP II) transcription and/or processing and/or histone H3 modification and/or chromatin remodeling include BAP1, CDK9, CDK7, ASXL2, REST, CCNT1, and/or SETD2.

In some embodiments, the LTF phenotype further includes reduced expression or reduced presence of one or more proteins selected from RNAP II Ser2, RNAP II Ser5, H3K4me3, H3K27me3, and H3K36me3. In some embodiments, the sample can have reduced expression or reduced presence of at least one of RNAP II Ser2 and/or RNAP II Ser5, and at least one of H3K4me3, and/or H3K27me3, and/or H3K36me3. In some embodiments, the sample can have reduced expression or reduced presence of both RNAP II Ser2 and RNAP II Ser5, and at least one of H3K4me3, and/or H3K27me3, and/or H3K36me3. In some embodiments, the sample can have reduced expression or reduced presence of at least one of RNAP II Ser2 and/or RNAP II Ser5, and at least two of H3K4me3, and/or H3K27me3, and/or H3K36me3. In some embodiments, the sample can have reduced expression or reduced presence of at least one of RNAP II Ser2 and/or RNAP II Ser5, and all three of H3K4me3, and/or H3K27me3, and/or H3K36me3. In some embodiments, the sample can have reduced expression or reduced presence of each of the RNAP II Ser2, RNAP II Ser5, H3K4me3, H3K27me3, and H3K36me3 proteins.

In some embodiments of the invention, the LTF phenotype further includes further include overexpression of PEA-15 protein and/or one or more protein synthesis pathway protein(s) and/or reduced expression of one or more proteins selected from NF-κB, EGFR, STAT3, STATS, MAPK, MEK1 (MAP2K1), and derivatives thereof, including phosphorylated derivatives thereof (e.g. phosphorylated MAPK, phosphorylated NF-κB), and inflammatory response proteins.

In some embodiments, the LTF phenotype further includes reduced expression of one or more aberrant or non-canonical mRNA isoforms selected from CCNT1, REST, ASXL2, KIF2A, PRKAR1A, NUP84, and NUP100, and/or overexpression of one or more aberrant or non-canonical mRNA isoforms selected from NDUFA3, NDUFA1, PFDN5, PFDN5, DGUOK, and MRPL11.

In some embodiments, the type of cancer includes one or more selected from cancers of the skin, breast, bladder, kidney, brain, head and neck, pancreas, prostate, liver, lung, ovary, blood, and colon.

In some embodiments of the methods, the subject can be treated based on the lack of suitability of immunotherapy where the tumor cells of the subject have an LTF phenotype, or based on the suitability of immunotherapy where the tumor cells of the subject lack an LTF phenotype. In some embodiments, the subject has the LTF phenotype, and the treatment does not include immunotherapy, but includes at least one of chemotherapy and/or targeted therapy and/or alternative therapy, provided that the targeted therapy is not an immunotherapy, or wherein the chemotherapy and/or targeted therapy includes at least one of sunitinib, everolimus, sirolimus, vemurafenib, and/or trametinib. In some embodiments, the subject lacks the LTF phenotype, and wherein the treatment includes immunotherapy. In some embodiments, the treatment further includes at least one of chemotherapy and/or targeted therapy and/or alternative therapy, or wherein the chemotherapy and/or targeted therapy includes at least one of sunitinib, everolimus, sirolimus, vemurafenib, and/or trametinib. In some embodiments, the immunotherapy includes administration of one or more interleukin, interferon (IFN), and/or small molecule indoleamine 2,3-dioxygenase (IDO) inhibitor, and/or one or more suitable antibody-based reagent, or one or more checkpoint inhibitory antibodies, including ipilimumab. In some embodiments, the immunotherapy includes administration of denileukin diftitox and/or administration of an antibody-based reagent selected from ado-trastuzumab emtansine, alemtuzumab, atezolizumab, bevacizumab, blinatumomab, brentuximab vedotin, cetuximab, catumaxomab, gemtuzumab, ibritumomab tiuxetan, ilipimumab, natalizumab, nimotuzumab, nivolumab, ofatumumab, panitumumab, pembrolizumab, rituximab, tositumomab, trastuzumab, and vivatuxin. In some embodiments, the treatment can be conducted as part of a clinical trial.

In some embodiments, the preferential expression or the higher proportion of the one or more aberrant or non-canonical mRNA isoforms can be that of one or more type I genes as defined herein.

In some embodiments, the one or more aberrant or non-canonical mRNA isoform(s) can include aberrant or non-canonical mRNA isoform(s) lacking exon sequences required for encoding a protein encoded by a corresponding normal or canonical mRNA isoform, including full-length isoforms. In some embodiments, the aberrant or non-canonical mRNA isoform(s) encode protein that is shorter than the corresponding full-length protein by an amount selected from less than 98%, less than 97%, less than 95%, less than 90%, less than 85%, less than 80%, less than 75%, less than 70%, and less than 60%.

Embodiments of the invention also encompass methods of stratifying one or more subjects in a clinical trial, including: analyzing, by RNA and/or protein analysis, a sample having tumor cells from one or more subject(s) having cancer to determine whether the tumor cells have a loss of transcriptional fidelity (LTF) phenotype, wherein the LTF phenotype is characterized by: having a preferential expression or higher proportion of one or more aberrant or non-canonical mRNA isoforms, relative to a control value for expression or proportion; and/or by reduced expression or reduced presence of one or more proteins selected from RNAP II Ser2, RNAP II Ser5, H3K4me3, H3K27me3, and H3K36me3 relative to a respective control value of expression or presence of RNAP II Ser2, RNAP II Ser5, H3K4me3, H3K27me3, and H3K36me3; and determining a lack of suitability of immunotherapy where the tumor cells of the subject have an LTF phenotype, or determining a suitability of immunotherapy where the tumor cells of the subject lack an LTF phenotype. In some embodiments, the control value for expression or proportion can be that of normal cells, that of non-LTF tumor cells, or that of mRNA corresponding to one or more internal control genes of the tumor cells not affected by LTF. In some embodiments, the one or more internal control genes of the tumor cells not affected by LTF, includes one or more type II genes as defined herein. In some embodiments, the control value of expression or presence of RNAP II Ser2, RNAP II Ser5, H3K4me3, H3K27me3, and H3K36me3 can be that of normal cells, or that of non-LTF tumor cells.

In some embodiments, in the context of a clinical trial, the subject can be treated based on the lack of suitability of immunotherapy where the tumor cells of the subject have an LTF phenotype, or based on the suitability of immunotherapy where the tumor cells of the subject lack an LTF phenotype.

Embodiments of the invention also encompass diagnostic kits, tests, or arrays to test for presence of a loss of transcriptional fidelity (LTF) phenotype in a sample, including: materials for quantification of phosphorylation at amino acid position RNAP II Ser2, and/or RNAP II Ser5; and/or materials for methylation analysis at amino acid position H3K4me3, H3K27me3, and H3K36me3 proteins; and/or materials for determining the presence or absence of transcriptional fidelity (LTF) phenotype characterized by having a preferential expression or higher proportion, relative to normal cells or to non-LTF tumor cells, of one or more aberrant or non-canonical mRNA isoform(s), relative to a control value. In some embodiments, the control value can be that of normal cells, that of non-LTF tumor cells, or that of mRNA corresponding to one or more internal control genes of the tumor cells not affected by LTF. In some embodiments, the one or more internal control genes of the tumor cells not affected by LTF, includes one or more type II genes as defined herein.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts the frequency of gene isoform occurrence.

FIG. 2A-2F. A) Expression characteristics of a gene at the level of its isoforms can be differentiated from its gene-level expression characteristics. B) Left: A 3-dimensional scatter plot of indicated expression parameters of genes. Every point in the plot represents a gene, represented by its ID value as indicated in the key. Right: 2-dimensional projections of the 3-dimensional plot on the left to indicated axes. C) All-against-all correlation heatmap matrices of (left) transcript isoforms from genes that are predicted to be regulated at the level of alternative transcription. Right: all-against-all expression correlation heatmap of gene-level (as opposed to transcript isoform-level) expression of the same genes. D) mRNA lengths of transcript isoforms in the clusters 1 and 2 in FIG. 2C (left panel). E) The transcript shortening (TS) phenotype observed in A-D is commonly observed in human cancers. KIRC: clear cell renal cell carcinoma, LUAD: lung adenocarcinoma, SKCM: skin cutaneous melanoma. Heatmaps on the left in each row show all-against-all expression correlations of mRNA isoforms of genes with alternative expression patterns. Boxplots on the right are same as in FIG. 2D for the indicated cancers and corresponding isoforms. F) Frequencies of occurrences of TS in various cancers.

FIG. 3A-3G shows that a subset of cancers is characterized by widespread loss of transcription fidelity. A. Relative expression level of short- and full-length transcript isoforms in 813 breast cancer samples. Relative isoform expression indicates relative expression of the given transcript isoform to the sum of expression values of all isoforms for its corresponding gene: 0 indicates that the given isoform is not being expressed by that gene, and 1 indicates that the given isoform is the only isoform being expressed for the given gene. The set of samples where the shorter isoforms of genes are dominantly expressed is underlined. B. Differential exon expression heatmap of 10,448 genes at the level of their exons. Difference in the expression of every exon between TS+ and TS-KIRC samples was calculated by t-test to obtain t-values of difference (t-statistic), and displayed in a heatmap format by dividing the exons of genes into 20 exon bins. The indicated gene sets (Types I-III) represent clusters of interest based on peculiar exon expression characteristics. C. Exon and intron coverage plots of RNA-seq reads from representative LTF+ and LTF− samples in KIRC for a STAT1 and a Type I gene (From Integrative Genome Viewer (Thorvaldsdottir et al., 2013)). Only a portion of the gene corresponding to the 3′ end is shown (top). The portion on top is shown in more detail on the bottom to highlight the early termination. D) Same as in (C), for TRAF1, highlighting poor exon definition in the 3′-most part of the gene. E) For each exon-exon junction (e-e), the corresponding exon-intron (e-i) and intron-exon junctions (i-e), and the ratios ([e-i+i-e]/e-e) were calculated. The boxplot shows the distributions of all such ratio values in TS+ and TS-KIRC samples for Type I (E) and Type II (F) genes. G) Median of genome-wide intron/exon ratio values for TS+ and TS-samples in KIRC. The p-value is for Wilcoxon test.

FIG. 4 shows intronic and spurious transcription in samples with TS. A) Exon and intron coverage of RNAseq reads of 3 representative TS+ and TS− samples from portions of indicated genes.

FIGS. 5A and 5B. A) Differential exon expression heatmaps in LTF+vs. LTF− cancers in BRCA, GBM and LUAD. The top lines to the left of the graphic in BRCA and LUAD represent Type I genes, while the bottom lines to the left of the graphic in BRCA and LUAD represent Type II genes. The top line to the left of the graphic in GBM represents Type II genes, while the bottom line to the left of the graphic in GBM represents Type II genes. B) Correlation heatmap of LTF mRNA signatures in different cancers. A LTF mRNA signature is the distribution of t-statistic values reflecting difference in the expression of every gene in LTF+vs. LTF− samples.

FIG. 6A-6H shows that LTF is observed in cell lines and involves defective mRNA transcription and splicing. A) Relative expression of truncated and full-length transcript isoforms in breast cancer cell lines (done the same way as in FIG. 3C). Two cell lines (UACC-812 and MDA-MB-415) with relatively increased expression of truncated isoforms are highlighted. B) Scatterplot of t-values of difference for every gene's expression in LTF+vs. LTF− samples in The Cancer Genome Atlas (TCGA), CCLE and our independent RNAseq data. Every point represents a gene, coloring reflects the t-values in TCGA BRCA samples. Pearson's r for correlation of t-values from TCGA and our RNAseq data is 0.40 (P<100-300). C) Western blots of RNAP II marks in indicated cell lines. D) Levels of mRNAs that are capped (according to m7G-mRNA pull-down) or uncapped in the indicated cell lines after depletion of rRNA. E) Levels of mRNAs that are poly-adenylated (according to oligo-dT pull-down) in the indicated cell lines. F) A network plot of some of the most consistently repressed genes in LTF+ cancers that are involved in chromatin remodeling and RNAP II-mediated transcription. G) Western blots of indicated histone marks and corresponding enzymes in the indicated cell lines. (H) A model of epigenetic and transcriptional defects in LTF. Histone modifications direct proper positioning and elongation of RNAP II along the gene and assembly of mRNA processing machinery (left). Loss of histone and DNA methylations in LTF leads to spurious transcription by RNAP II and improper mRNA processing (right). The error bars in D and E are S.D. of triplicate measurements, and are representative of two independent experiments.

FIG. 7A-7D. A) Differential exon expression heatmap of LTF+ and LTF− breast cancer cell lines from Cancer Cell Line Encyclopedia (CCLE) RNAseq data. Produced the same way as in FIG. 3B. B) Intron/Exon expression ratios of all expressed genes in the indicated breast cancer cell lines, based on RNAseq data from CCLE. Potential LTF+ cell lines (based on analyses in FIG. 6A-C) are indicated. C) Correlation of differential genomewide expression signature of UACC-812 and MDA-MB415 cells with the LTF signature in different cancers from TCGA. D) Intron retention ratio (calculated same way as in FIG. 3E) for Type I genes based on our independent RNAseq data.

FIG. 8. Differential chromatin mark enrichment profiles of down- and up-regulated genes (Type I and II, respectively) in LTF+ cancers in promoter (−1k:+1), exon and intron regions of genes (see method and materials section, following the examples). The heatmap shows the marks with the most significant difference in enrichment (difference in the z-score of enrichment). Zup: z-score of enrichment in up-regulated genes (Type II). Notice the enrichment of up-regulated genes for active chromatin and related marks (e.g. H2A.Z, POLR2A, histone acetylations), while down-regulated genes (Type I) are enriched for poised promoters, characterized by repressive (e.g. H3K27me3) and activating marks.

FIG. 9A-9F shows that LTF affects long gene expression and pathway activity. A) Gene length distributions of Type I, II and III genes from FIG. 3B. B) Gene length distributions of pathways that are most enriched in Type I (in bold rectangles) and Type II (in regular, non-bold rectangles) genes. C) Expression difference of every protein between LTF+ and LTF− samples in the indicated cancer datasets was calculated by t-test using the reverse-phase protein array (RPPA) data. The figure shows the clustered heatmap of the resultant t-values. Proteins with consistent down- and up-regulation in LTF+ tumors are highlighted (clusters 1 and 2, respectively). D) Gene, mRNA and protein lengths of proteins in clusters 1 and 2 in (C). Only total (i.e. not post-translationally modified) proteins were included in the boxplots. The p-values reflect Wilcoxon test. E) Some of the most significantly and consistently altered proteins in LTF+ cancers. F) Western blots of indicated proteins in the indicated cell lines.

FIG. 10A-10B. Correlation of expression differences of individual exon-exon junctions in LTF+vs. LTF− samples with the corresponding intron gaps between the exons. a) An illustration of the concept: RNAP II that has low fidelity will transcribe long DNA segments less efficiently, manifesting in less coverage of the exon-exon junctions spanning longer introns. Importantly, this analysis is independent of the mRNA length and only depends on the DNA length, which is important to exclude the possibility of mRNA degradation in the LTF phenotype. B) Distribution of intron lengths of exon-exon junctions classified based on the t-statistic of difference of the junction expression between LTF+ and LTF samples in KIRC. Only the most terminal exon-exon junction for each gene was included in this analysis.

FIG. 11A-11C. A) Pathway enrichment profiles of Type I (solid black) and Type II (grey) genes in the indicated cancers. X-axes show p-values of enrichment (−log 10) from hypergeometric distribution. B) Heatmap of correlations of LTF protein signatures in different cancers. A LTF protein signature is the distribution of t-statistic values of difference of every protein measured in RPPA data between LTF+vs. LTF− samples. C) The LTF protein signature of LTF+ breast cancer cell lines (UACC-812 and MDA-MB-415). The x-axis shows the significance of change. For example, −5 means a given protein is down-regulated in LTF+ cells relative to all other breast cancer cell lines with a p-value of P=10-5, while 5 would indicate upregulation with the same p-value. The most significant (P<=0.01) differences are shown. Data are based on published RPPA data for breast cancer cell lines.

FIG. 12A-12C shows that LTF weakly correlates with mutations in some histone modifiers in KIRC. A) LTF+ fraction in KIRC patients with and without BAP1 mutations. B) Boxplots of SETD2 mRNA (left) and protein (right, based on RPPA data) levels in KIRC samples with no or indicated SETD2 mutations. P values reflect multivariate linear regression. c) Fraction of tumors with indicated SETD2 mutations that are also GCT+ in KIRC. P-value reflects Fisher's exact test. C) Immunoblots of indicated histone and RNAP II marks in bone marrow cells from Setd2 wild type, missense mutation knock-in and hemizygous knock-out mice.

FIG. 13A-13H shows that LTF confers clinical resistance to immunotherapy. A) Kaplan-meier survival curves of LTF+ and LTF− KIRC patients. Left: all patients, middle: all patients that received treatment, right: those that received immunotherapy. B) Same as in (A) in SKCM patients. Middle: patients treated with immunotherapy other than immune checkpoint inhibitors, right: patients that received immune checkpoint inhibitor therapy. The p-values reflect log-rank test. C) Left: LTF was scored in the RNAseq samples from 42 ipilimumab-treated melanoma patients as global retention of exon-intron junctions in Type I genes (same as in FIG. 3G), and were compared between responding, non-responding and long-survival patient groups, as defined in the original study. Right: Kaplan-meier curves for PFS and OS in LTF+ and LTF− patients. D) Kaplan-meier curves of OS and PFS in the same patients stratified according to LTF and tumor infiltration by lymphocytes (TIL) status (see Methods). E) A diagram of CTL/NK-Tumor cell interaction through FasL/Fas signaling. F) Levels of cleaved Caspase 7 (measured by RPPA) in KIRC and SKCM samples stratified by LTF and GZMB expression (*: P<0.05; **: P<0.01). G) Relative viability of indicated cell lines after 24 hour FasL treatment. H) Immunoblot of Caspase 8 and Caspase 3 levels in the indicated cell lines. The Caspase 3 blot was later probed with the GAPDH antibody. H) Caspase 8 activity levels in indicated cell lines before and after stimulation with FasL for 6 hours. The error bars in this figure reflect S.D. of at least 3 replicate conditions.

FIGS. 14A and 14B. A) LTF+ KIRC patients respond better to targeted therapy compared to immunotherapy. Kaplan-meier survival curves of LTF+(right) and LTF− (left) patients that were treated with immunotherapy or targeted therapy. B) Immune infiltration in LTF+ tumors. Difference in the expression of indicated marker genes for cytotoxic T lymphocytes and natural killer cells was calculated by t-test in KIRC and SKCM. Heatmap colors show −log 10 P values of difference with the sign indicating direction of difference (i.e. negative: reduced; positive: increased, expression in LTF+ tumors). Some of the genes' common names are indicated on the right.

FIG. 15A-15K shows that loss of gene body histone methylation or transcription elongation causes LTF-like defects in transcription and immune response. A) Immunoblots of indicated histone and RNAP II marks in T47D and Cal51 cells with and without SETD2 knock-down (shSETD2). B) Relative levels of capped and uncapped mRNAs in indicated cell lines with and without stable shSETD2. C) Relative levels of poly-adenylated mRNAs in equal amount of total RNA in indicated cells with and without shSETD2. D) Immunoblot of indicated proteins showing response to TNF-α and IFN-α stimulations in shSETD2 and control cells. E) Relative viability of indicated cell lines after 24 hours of treatment with FasL (10 ng/mL). F) Caspase 8 activity levels in indicated cells before and after FasL treatment for 6 hours. G) RNAP II and histone marks in Cal51 and T47D cells treated with increasing doses of flavopiridol for 48 hours. H) STAT1 activation levels in Cal51 cells treated with indicated doses of flavopiridol for 48 hours, and treated with IFN-α for 30 minutes. I) Relatively viability of Cal51 cells after 24 hours of FasL stimulation, with and without 48 hour pre-treatment with 100 μM flavopiridol. J) In vivo assessment of resistance to NK-mediated anti-tumor response: mice are injected intravenously with 2×10⁵ B16/F10 cells constitutively expressing the chick ovalbumin (OVA) gene (B16-OVA). Bottom: Relative levels of OVA (normalized to GAPDH) in the lungs of mice after 1 hour of injection were measured by qPCR under the indicated 4 conditions. ΔNK:NK cell depletion by subcutaneous pre-injection of mice with anti-asialo GM antibody 1 day prior to tumor cell injection. K) A model of the role of intact epigenetic and transcriptional fidelity in the tumor cell response to anti-tumor immune attacks and immunotherapy. Error bars in this figure (except in (J)): S.D. of triplicate measurements, representative of at least 2 independent experiments. In (J), the error bars reflect S.D. of 6 replicates per group.

FIG. 16. Correlation of protein levels of different cleaved caspases (based on RPPA data) with cytolytic lymphocyte infiltration (based on GZMB expression) in different cancers. ***: P<10-10, *: P<0.05.

FIG. 17. Indicated cells were stimulated with IFN-α or TNF-α for 30 minutes, and assessed for the indicated response markers.

### DETAILED DESCRIPTION OF THE INVENTION

Unless otherwise noted, terms are to be understood according to conventional usage by those of ordinary skill in the relevant art.

As used herein, the term “sample” encompasses a sample obtained from a subject or patient. The sample can be of any biological tissue or fluid and can be fresh, frozen, or otherwise preserved (e.g. paraffin-embedded). Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue (including cancerous tissue, tumor tissue, etc.), core or fine needle biopsy samples, cell-containing body fluids, free floating nucleic acids, urine, stool, peritoneal fluid, and pleural fluid, liquor cerebrospinalis, tear fluid, or cells therefrom. Samples can also include sections of tissues such as frozen or fixed sections taken for histological purposes or microdissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human (including a human having cancer/tumor).

As used herein, the term “diagnosing” or “monitoring” with reference to a disease state or condition refers to a method or process of determining if a subject has or does not have a particular disease state or condition or determining the severity or degree of the particular disease state or condition.

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition. The term “treatment” is used in some embodiments to refer to administration of a compound of the present invention to mitigate a disease or a disorder in a host, preferably in a mammalian subject, more preferably in humans. Thus, the term “treatment” can include includes: preventing a disorder from occurring in a host, particularly when the host is predisposed to acquiring the disease, but has not yet been diagnosed with the disease; inhibiting the disorder; and/or alleviating or reversing the disorder. Insofar as the methods of the present invention are directed to preventing disorders, it is understood that the term “prevent” does not require that the disease state be completely thwarted (see Webster's Ninth Collegiate Dictionary). Rather, as used herein, the term preventing refers to the ability of the skilled artisan to identify a population that is susceptible to disorders, such that administration of the compounds of the present invention can occur prior to onset of a disease. The term does not mean that the disease state must be completely avoided.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The terms “over-expressed”, “highly expressed”, “high expression”, “under-expressed”, and “low expression” refer to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain pattern or combination of expression levels can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the invention, a reference pattern of expression levels is, for example, an average pattern of expression levels observed in a group of healthy or diseased individuals, serving as a reference group.

As used herein, the term “canonical”, in the context of a sequence of residues, for example, residues of nucleotides, amino acids, and the like, refers to the most commonly found sequence at the respective positions. Such canonical sequences can therefore be used as reference sequences when determining whether a sample sequence differs relative to a corresponding canonical sequence(s), of when determining whether a sample sequence is an aberrant or non-canonical sequence.

As used herein, an “aberrant” sequence is one which differs in any way from the corresponding canonical sequence. Such aberrant sequences can differ in individual residues, in folding, in length, etc.

As used herein, an mRNA “isoform” is an alternative transcript for a specific mRNA or gene. This term includes pre-mRNA, immature mRNA, mature mRNA, cleaved or otherwise truncated, shortened, or aberrant mRNA, modified mRNA (e.g. containing any residue modifications, capping variants, polyadenylation variants, etc.), and the like.

“Antibody” or “antibody peptide(s)” refer to an intact antibody, or a binding fragment thereof that competes with the intact antibody for specific binding; this definition also encompasses monoclonal and polyclonal antibodies. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody, for example, substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

### Loss of Transcriptional Fidelity (LTF) in Cancers

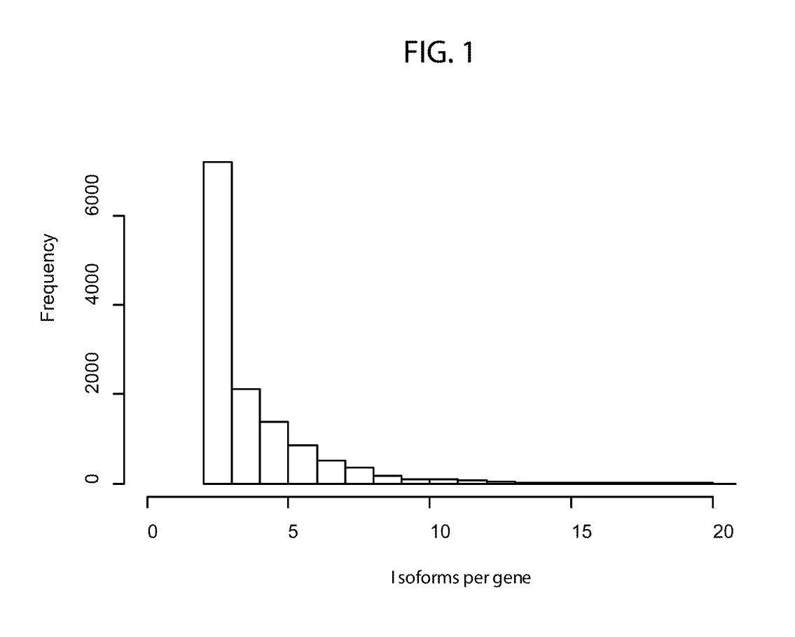

Greater than 90% of human genes have been found to have alternative transcripts (FIG. 1), or isoforms. Genes that are subject to regulation at the level of transcript (isoform) switching are usually long genes (>10 exons), and involve genes in certain pathways, such as mRNA splicing/processing, chromatin remodeling and inflammatory pathways. A phenotype with widespread spurious transcription and mRNA processing defects is described herein as “Loss of Transcriptional Fidelity” (LTF).

The alternative transcripts of long genes are coordinately regulated in cancers, but not normal tissues. Mutations in the core epigenetic and transcriptional machinery can have more widespread effects than sequence-specific transcription factors, potentially deregulating transcription at the genome level. For example, such widespread defects in mRNA transcription, splicing and poly-adenylation have been reported in kidney tumors with mutations in SETD2, a key enzyme in the tri-methylation of H3 histones at lysine 36 within gene bodies (Simon et al., 2014). It is, therefore, clear that at least some cancers have widespread defects in their epigenetic and transcriptional programs, perhaps reflecting a tumorigenic advantage of such global deregulations. Indeed, widespread 3′ shortening of untranslated regions (UTRs) in cancers due to alternative poly-adenylation has been shown to allow tumor cells to escape miRNA-mediated repression of oncogenic pathways (Mayr and Bartel, 2009). Recent studies have also uncovered widespread deregulations in the transcriptional and mRNA splicing processes that did not necessarily correlate with any known somatic mutations (Dvinge and Bradley, 2015; Sowalsky et al., 2015), indicating the non-genetic origin of some core transcriptional and splicing defects in cancers. Overall, although much has been learnt on the mechanisms of transcription and post-transcriptional mRNA processing, the nature, mechanisms and clinical consequences of their aberrations in cancers have heretofore not been fully understood.

As described herein, a comprehensive analysis of aberrant alternative transcription events in human cancers was conducted. The mRNA sequencing datasets from The Cancer Genome Atlas (TCGA) were used to provide an unprecedented interrogation regarding aberrant transcription events in human cancers and assessment of their clinical relevance. To identify most prominent and widespread aberrant transcription events in human cancers, a pan-cancer analysis of the TCGA mRNA-seq datasets was performed. The RNA-seq datasets contain information for >25 cancers, with separate gene-, exon-, junction- and transcript-level quantitation of expression. These data were analyzed for global mRNA splicing errors.

Some cancers were found to have severe loss of epigenetic and mRNA transcriptional fidelity, characterized by widespread spurious transcription and mRNA processing defects (i.e. “Loss of Transcriptional Fidelity”, or LTF). Close to 10% of all human cancers were characterized by severely defective genic histone methylations as well as transcriptional and mRNA processing machineries, resulting in widespread defects in the transcription of long genes, i.e. truncated transcripts, including preferential expression of only terminal exons for a large number of genes.

Importantly, these transcriptional defects had a highly specific impact on the functional landscape of these tumors, which led to impaired response to pro-inflammatory death stimuli, resistance to immune-mediated attacks and, consequently, to immunotherapy in the clinic. Because LTF impairs transcriptional elongation and imposes a highly specific molecular phenotype where pathways regulated by long genes, such as those involved in the inflammatory response, are consistently impaired in LTF+(i.e. those with LTF) tumors, LTF+ cancer patients have specific poor response to immunotherapeutic drugs, drugs in renal cell carcinoma and melanoma patients.

Genetic or chemical perturbation of the gene body histone methylation or of transcriptional elongation can recapitulate LTF-like widespread epigenetic, transcriptional and mRNA processing defects, impair cellular response to pro-inflammatory stimuli, and impose resistance to immune-mediated anti-tumor mechanisms in vitro and in vivo. Therefore, severe epigenetic and transcriptional defects in a subset of cancers confers resistance to anti-tumor immune attacks.

### LTF Phenotype

The studies detailed herein describe LTF as a previously unknown clinically significant phenotype in cancers and demonstrate a clinically significant novel subclass of human tumors with specific pathway activation and therapeutic response profiles. LTF can therefore be utilized in cancer patients for proper assignment of therapy, particularly therapies involving immunotherapy. In particular, LTF can be assessed in cancer patients undergoing immunotherapy in order to determine and/or predict response.

In some embodiments of the invention, an LTF phenotype can be characterized by having a preferential expression or higher proportion of one or more aberrant or non-canonical mRNA isoforms, relative to a control value. For example, in some embodiments, the control value can be that of normal cells, that of non-LTF tumor cells, or that of mRNA corresponding to one or more internal control genes of the tumor cells not affected by LTF. In some embodiments, the one or more internal control genes of the tumor cells not affected by LTF, can include one or more type II genes.

In some embodiments, the aberrant or non-canonical mRNA isoforms include aberrant or non-canonical mRNA isoforms lacking exon and/or intron sequences found in the corresponding normal or canonical mRNA isoforms, including full-length isoforms, or retaining exon and/or intron sequences not found in the corresponding normal or canonical mRNA isoforms, including full-length isoforms. In some embodiments, the one or more aberrant or non-canonical mRNA isoforms include aberrant or non-canonical mRNA isoforms lacking 5′-exon sequences found in the corresponding normal or canonical mRNA isoforms, including full-length isoforms, or retaining 5′exon sequences not found in the corresponding normal or canonical mRNA isoforms, including full-length isoforms. In some embodiments, the one or more aberrant or non-canonical mRNA isoforms include aberrant or non-canonical mRNA isoforms having an increased amount of retained intron-exon junctions compared to the corresponding normal or canonical mRNA isoform(s), including full-length isoforms. In some embodiments, the one or more aberrant or non-canonical mRNA isoforms include aberrant or non-canonical mRNA isoforms lacking exon sequences required for encoding a protein encoded by a corresponding normal or canonical mRNA isoform including full-length mRNA isoforms thereof.

In some embodiments, an LTF phenotype can be characterized by reduced expression or reduced presence of one or more proteins selected from the group consisting of RNAP II Ser2, RNAP II Ser5, H3K4me3, H3K27me3, and H3K36me3 relative to a respective control value. For example, in some embodiments, the control value can be that of normal cells, or that of non-LTF tumor cells.

In some embodiments, the sample has reduced expression or reduced presence of: at least one of RNAP II Ser2 and/or RNAP II Ser5, and at least one of H3K4me3, and/or H3K27me3, and/or H3K36me3; or of both RNAP II Ser2 and RNAP II Ser5, and at least one of H3K4me3, and/or H3K27me3, and/or H3K36me3; or of at least one of RNAP II Ser2 and/or RNAP II Ser5, and at least two of H3K4me3, and/or H3K27me3, and/or H3K36me3; or at least one of RNAP II Ser2 and/or RNAP II Ser5, and all three of H3K4me3, and/or H3K27me3, and/or H3K36me3; or of each of the RNAP II Ser2, RNAP II Ser5, H3K4me3, H3K27me3, and H3K36me3.

In some embodiments, the LTF phenotype includes a preferential expression or higher proportion, relative to that of normal cells, to that of non-LTF tumor cells, or to that of mRNA corresponding to one or more internal control genes of the tumor cells not affected by LTF, of one or more aberrant or non-canonical mRNA isoforms of corresponding normal or canonical mRNA isoforms, including full-length isoforms.

In some embodiments, an LTF phenotype can be characterized by having both: a) a preferential expression or higher proportion of one or more aberrant or non-canonical mRNA isoforms, relative to a control value, and b) reduced expression or reduced presence of one or more proteins selected from the group consisting of RNAP II Ser2, RNAP II Ser5, H3K4me3, H3K27me3, and H3K36me3 relative to a respective control value.

In some embodiments, the sample can be processed to obtain RNAseq data. In some embodiments, the RNAseq data can be poly-A-selected RNAseq data or total RNAseq data. In embodiments involving poly-A-selected RNAseq data, the one or more aberrant or non-canonical pre-mRNA and/or mRNA isoform(s) can include non-canonical pre-mRNA and/or mRNA isoform(s) lacking 5′-exon sequences found in the corresponding normal or canonical pre-mRNA and/or mRNAs, including full-length isoforms, and/or the one or more aberrant or non-canonical pre-mRNA and/or mRNA isoform(s) can include normal or non-canonical pre-mRNA and/or mRNA isoform(s) having an increased amount of retained intron-exon junctions. In embodiments involving total RNAseq data, the one or more aberrant or non-canonical pre-mRNA and/or mRNA isoform(s) can include normal or non-canonical pre-mRNA and/or mRNA isoform(s) having an increased amount of retained intron-exon junctions.

In some embodiments, the aberrant or non-canonical mRNA isoform(s) encode one or more protein(s) that are shorter than the corresponding full-length protein. For example, in some embodiments, the shortened protein can be shorter than the corresponding full-length protein by an amount selected from the group consisting of less than 98%, less than 97%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 79%, less than 78%, less than 77%, less than 76%, less than 75%, less than 74%, less than 73%, less than 72%, less than 71%, less than 70%, less than 65%, less than 60%, less than 55%, less than 50%, less than 45%, less than 40%, less than 35%, less than 30%, and less than 25%. In some embodiments, the aberrant or non-canonical mRNA isoforms correspond to type I genes, as defined in Table 1 herein. Accordingly, in some embodiments, the one or more protein(s) that are shorter than the corresponding full-length protein relate to the products of the respective corresponding type I genes.

In some embodiments, for a given mRNA, a large portion or majority of the mRNA is present as corresponding aberrant or non-canonical mRNA isoforms. For example, in some embodiments, for a given mRNA, greater than 10%, greater than 11%, greater than 12%, greater than 13%, greater than 14%, greater than 15%, greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, greater than 40%, greater than 41%, greater than 42%, greater than 43%, greater than 44%, greater than 45%, greater than 46%, greater than 47%, greater than 48%, greater than 49%, greater than 50%, greater than 51%, greater than 52%, greater than 53%, greater than 54%, greater than 55%, greater than 56%, greater than 57%, greater than 58%, greater than 59%, greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99% of the mRNA can be present as corresponding aberrant or non-canonical mRNA isoforms. In some embodiments, the aberrant or non-canonical mRNA isoforms correspond to type I genes, as defined in Table 1 herein. Accordingly, in some embodiments, for a given type I gene mRNA, a large portion or majority of the mRNA is present as corresponding aberrant or non-canonical mRNA isoforms.

In some embodiments, for a given mRNA, a large portion or a majority of the mRNA expression is of corresponding aberrant or non-canonical mRNA isoforms. For example, in some embodiments, for a given mRNA, greater than 10%, greater than 11%, greater than 12%, greater than 13%, greater than 14%, greater than 15%, greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, greater than 40%, greater than 41%, greater than 42%, greater than 43%, greater than 44%, greater than 45%, greater than 46%, greater than 47%, greater than 48%, greater than 49%, greater than 50%, greater than 51%, greater than 52%, greater than 53%, greater than 54%, greater than 55%, greater than 56%, greater than 57%, greater than 58%, greater than 59%, greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99% of the mRNA expression can be of corresponding aberrant or non-canonical mRNA isoforms. In some embodiments, the aberrant or non-canonical mRNA isoforms correspond to type I genes, as defined in Table 1 herein. Accordingly, in some embodiments, a large portion or a majority of the mRNA expression for type I genes is of corresponding aberrant or non-canonical mRNA isoforms.

In some embodiments, a large portion or a majority of total mRNA is present as aberrant or non-canonical mRNA isoforms. For example, in some embodiments, greater than 10%, greater than 11%, greater than 12%, greater than 13%, greater than 14%, greater than 15%, greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, greater than 40%, greater than 41%, greater than 42%, greater than 43%, greater than 44%, greater than 45%, greater than 46%, greater than 47%, greater than 48%, greater than 49%, greater than 50%, greater than 51%, greater than 52%, greater than 53%, greater than 54%, greater than 55%, greater than 56%, greater than 57%, greater than 58%, greater than 59%, greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99% of total mRNA can be present as aberrant or non-canonical mRNA isoforms. In some embodiments, a large portion or a majority of total type I gene mRNA is present as aberrant or non-canonical mRNA isoforms.

In some embodiments, the one or more aberrant or non-canonical mRNA isoforms correspond to long genes. For example, in some embodiments, the one or more aberrant or non-canonical mRNA isoforms can correspond to normal or canonical mRNAs, including full-length mRNAs, having lengths of greater than 10 kb (kilobase pairs), greater than 25 kb, greater than 30 kb, greater than 35 kb, greater than 40 kb, greater than 345 kb, greater than 50 kb, greater than 60 kb, greater than 70 kb, greater than 75 kb, greater than 80 kb, greater than 90 kb, greater than 100 kb, greater than 110 kb, greater than 120 kb, greater than 130 kb, greater than 140 kb, greater than 150 kb, greater than 160 kb, greater than 170 kb, greater than 180 kb, greater than 190 kb, greater than 200 kb, greater than 225 kb, or greater than 250 kb.

In some embodiments, the aberrant or non-canonical mRNA isoforms have retained intron-exon junctions. For example, in some embodiments, greater than 5%, greater than 10%, greater than 11%, greater than 12%, greater than 13%, greater than 14%, greater than 15%, greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, greater than 40%, greater than 41%, greater than 42%, greater than 43%, greater than 44%, greater than 45%, greater than 46%, greater than 47%, greater than 48%, greater than 49%, greater than 50%, greater than 51%, greater than 52%, greater than 53%, greater than 54%, greater than 55%, greater than 56%, greater than 57%, greater than 58%, greater than 59%, greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99% of aberrant or non-canonical mRNA can have one or more retained intron-exon junctions.

In some embodiments, the mRNA has retained a large portion or a majority of intron-exon junctions. For example, in some embodiments, greater than 5%, greater than 10%, greater than 11%, greater than 12%, greater than 13%, greater than 14%, greater than 15%, greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, greater than 40%, greater than 41%, greater than 42%, greater than 43%, greater than 44%, greater than 45%, greater than 46%, greater than 47%, greater than 48%, greater than 49%, greater than 50%, greater than 51%, greater than 52%, greater than 53%, greater than 54%, greater than 55%, greater than 56%, greater than 57%, greater than 58%, greater than 59%, greater than 60%, greater than 61%, greater than 62%, greater than 63%, greater than 64%, greater than 65%, greater than 66%, greater than 67%, greater than 68%, greater than 69%, greater than 70%, greater than 71%, greater than 72%, greater than 73%, greater than 74%, greater than 75%, greater than 76%, greater than 77%, greater than 78%, greater than 79%, greater than 80%, greater than 81%, greater than 82%, greater than 83%, greater than 84%, greater than 85%, greater than 86%, greater than 87%, greater than 88%, greater than 89%, greater than 90%, greater than 91%, greater than 92%, greater than 93%, greater than 94%, greater than 95%, greater than 96%, greater than 97%, greater than 98%, or greater than 99% of intron-exon junctions can be retained compared to the corresponding normal or canonical mRNA isoforms, including full-length isoforms.

In some embodiments, the retained intron-exon junctions can be expressed as a ratio of intron-exon to exon-exon junctions, or vice versa (i.e. the ratio can be reversed). For example, intron to exon expression ratios can be calculated for a given gene by taking the ratio of total intron expression to that of exon expression. For example, for each exon-exon junction (e-e), and corresponding exon-intron (e-i) and intron-exon junctions (i-e), the exon-intron junction inclusion ratio can be calculated as ([e-i+i-e]/e-e). For example, in some embodiments, the exon-intron junction inclusion ratio of the aberrant or non-canonical mRNA isoform is greater than 0.01, greater than 0.011, greater than 0.012, greater than 0.013, greater than 0.014, greater than 0.015, greater than 0.016, greater than 0.017, greater than 0.018, greater than 0.019, greater than 0.020, greater than 0.021, greater than 0.022, greater than 0.023, greater than 0.024, greater than 0.025, greater than 0.026, greater than 0.027, greater than 0.028, greater than 0.029, greater than 0.030, greater than 0.031, greater than 0.032, greater than 0.033, greater than 0.034, greater than 0.035, greater than 0.036, greater than 0.037, greater than 0.038, greater than 0.039, greater than 0.040, greater than 0.041, greater than 0.042, greater than 0.043, greater than 0.044, greater than 0.045, greater than 0.046, greater than 0.047, greater than 0.048, greater than 0.049, greater than 0.050, greater than 0.051, greater than 0.052, greater than 0.053, greater than 0.054, greater than 0.055, greater than 0.056, greater than 0.057, greater than 0.058, greater than 0.059, greater than 0.060, greater than 0.061, greater than 0.062, greater than 0.063, greater than 0.064, greater than 0.065, greater than 0.066, greater than 0.067, greater than 0.068, greater than 0.069, greater than 0.070, greater than 0.071, greater than 0.072, greater than 0.073, greater than 0.074, greater than 0.075, greater than 0.076, greater than 0.077, greater than 0.078, greater than 0.079, greater than 0.080, greater than 0.081, greater than 0.082, greater than 0.083, greater than 0.084, greater than 0.085, greater than 0.086, greater than 0.087, greater than 0.088, greater than 0.089, greater than 0.090, greater than 0.091, greater than 0.092, greater than 0.093, greater than 0.094, greater than 0.095, greater than 0.096, greater than 0.097, greater than 0.098, greater than 0.099, greater than 0.10, greater than 0.11, greater than 0.12, greater than 0.13, greater than 0.14, greater than 0.15, greater than 0.16, greater than 0.17, greater than 0.18, greater than 0.19, greater than 0.20, greater than 0.25, greater than 0.30, greater than 0.35, greater than 0.40, greater than 0.45, or greater than 0.50, wherein the exon-intron junction inclusion ratio can be calculated as ([e-i+i-e]/e-e).

In some embodiments, the one or more aberrant or non-canonical mRNA isoform mRNA isoforms are encoded by one or more corresponding genes associated with RNA polymerase II (RNAP II) (e.g., GenBank Accession No. AAD05361; GI: 1220358; SEQ ID NO: 1) and/or histone H3 (e.g., GenBank Accession No. AAN39284; GI: 23664260; SEQ ID NO: 2). For example, in some embodiments, the one or more aberrant or non-canonical mRNA isoforms correspond to genes involved in RNAP II transcription and/or processing, H3 modification, chromatin remodeling, and the like. Such genes include, for example, BAP1, CDK9, CDK7, ASXL2, REST, CCNT1, and/or SETD2, and the like. For example, the RNAP II genes can include genes involved in RNAP II phosphorylation, and/or the genes involved in histone H3 modification and/or chromatin remodeling can include genes in involved in histone H3 methylation and/or acetylation. Genes involved in RNAP II phosphorylation include genes involved in RNAP II phosphorylation at amino acid positions Ser2 and/or Ser5, and the like. Genes involved in histone H3 methylation include genes involved in histone H3 methylation at amino acid positions K4, K27, and/or K36, and the like.

An LTF phenotype can also include reduced expression of corresponding full-length proteins. For example, the under-expressed full length proteins can include RNAP II Ser2, RNAP II Ser5, H3K4me3, H3K27me3, and H3K36me3, NF-κB, EGFR, STAT3, STATS, MAPK, MEK1 (MAP2K1), and derivatives thereof, particularly phosphorylated derivatives thereof (e.g. phosphorylated MAPK, phosphorylated NF-κB), and inflammatory response proteins. In some embodiments, 1, 2, 3, 4, or 5 of the full length proteins RNAP II Ser2, RNAP II Ser5, H3K4me3, H3K27me3, and H3K36me3 can have reduced expression. In some embodiments, certain full-length proteins can be overexpressed. For example, the over-expressed full length proteins can include PEA-15 protein and/or one or more protein synthesis pathway protein(s), and the like. In some embodiments, 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 75 or more, 100 or more, 150 or more, 200 or more, 250 or more, 300 or more, 350 or more, 400 or more, 450 or more, or more than 500 full-length proteins can have reduced or increased expression, associated with an LTF phenotype.

### LTF in Cancer Treatment

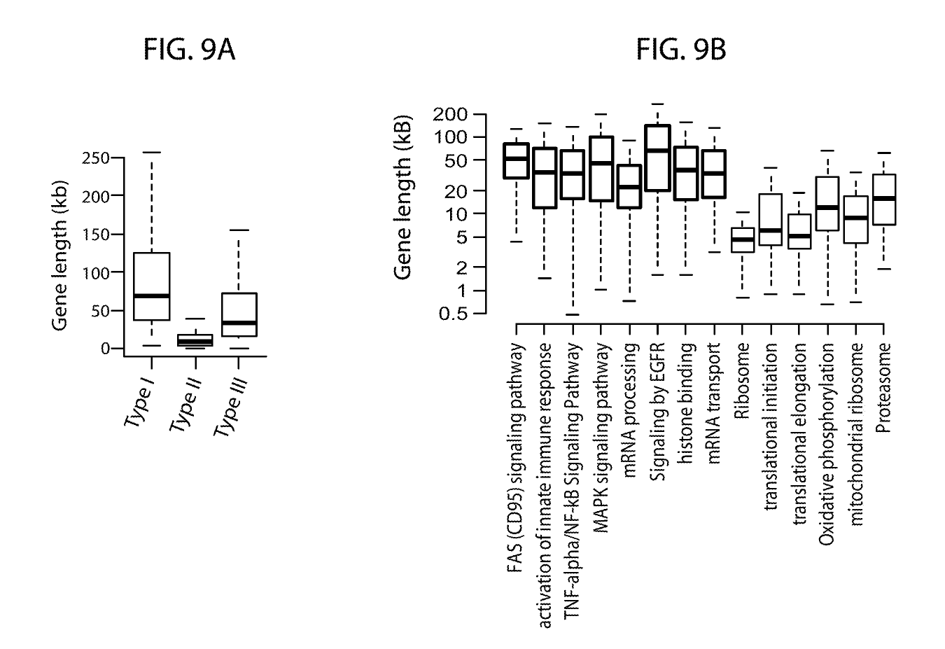

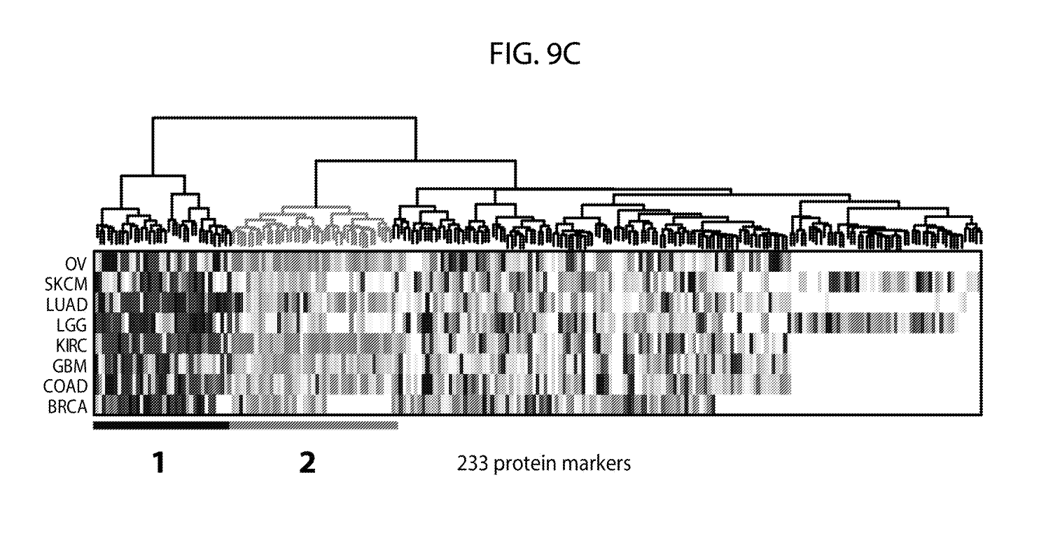

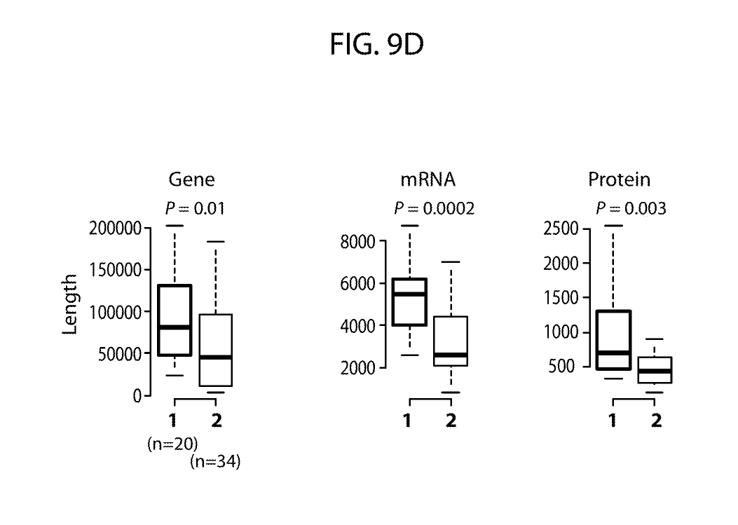

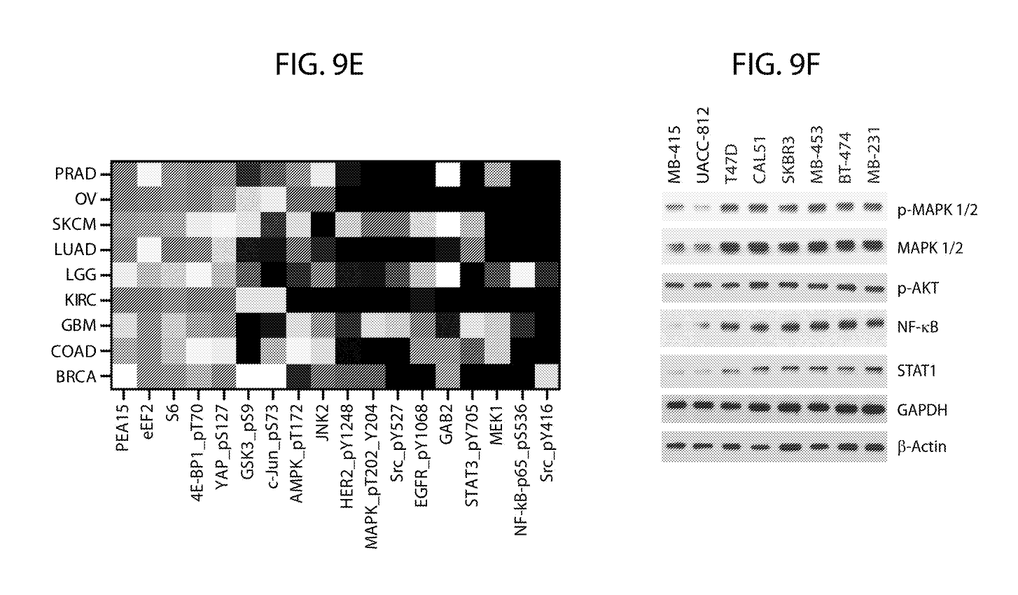

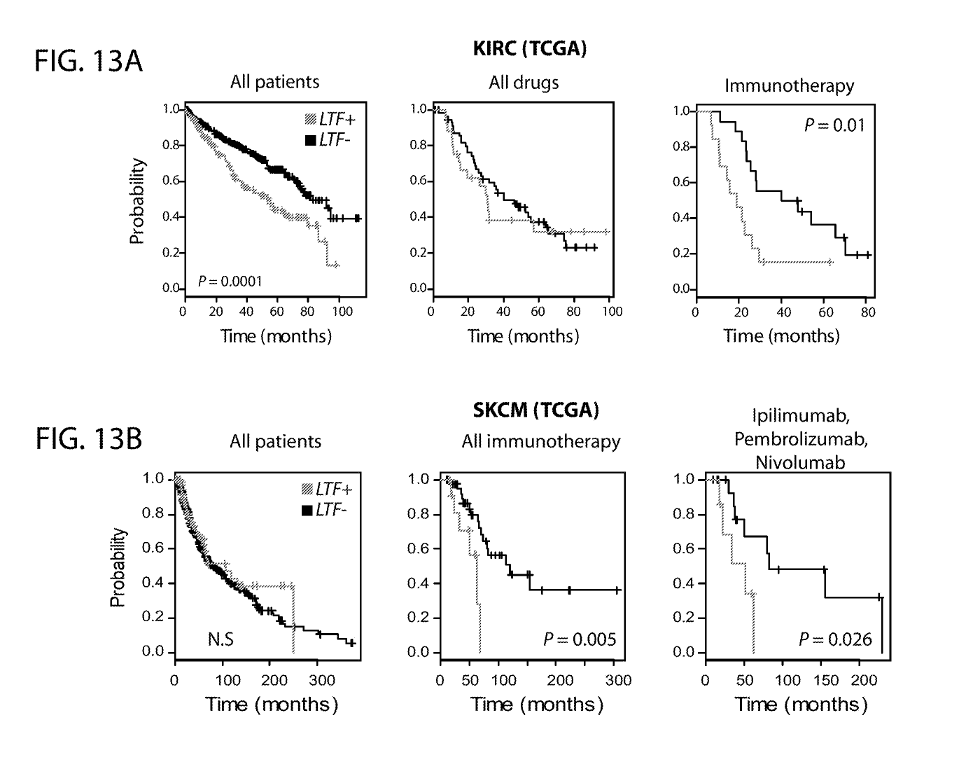

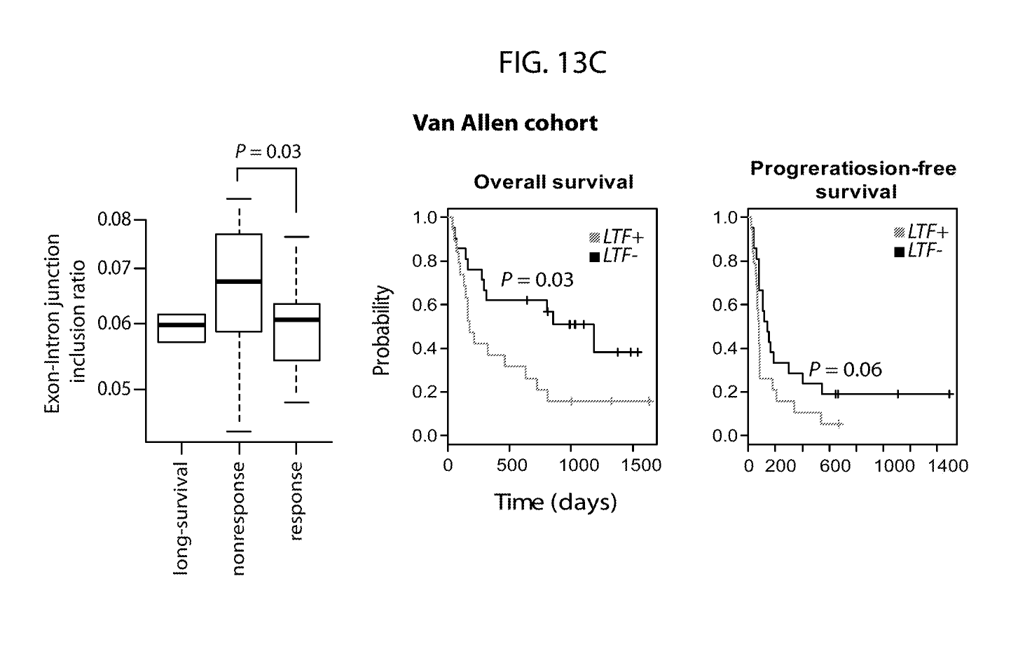

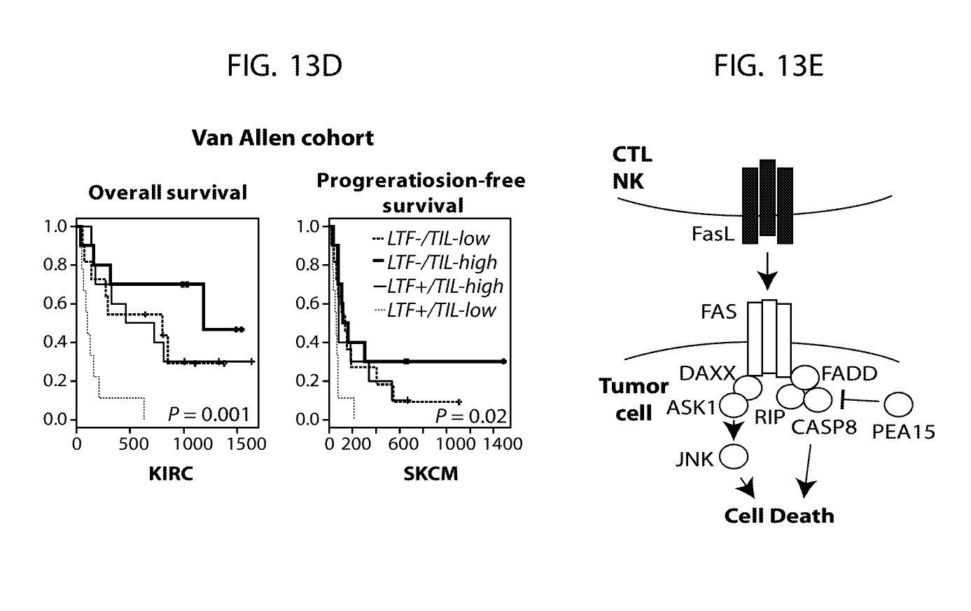

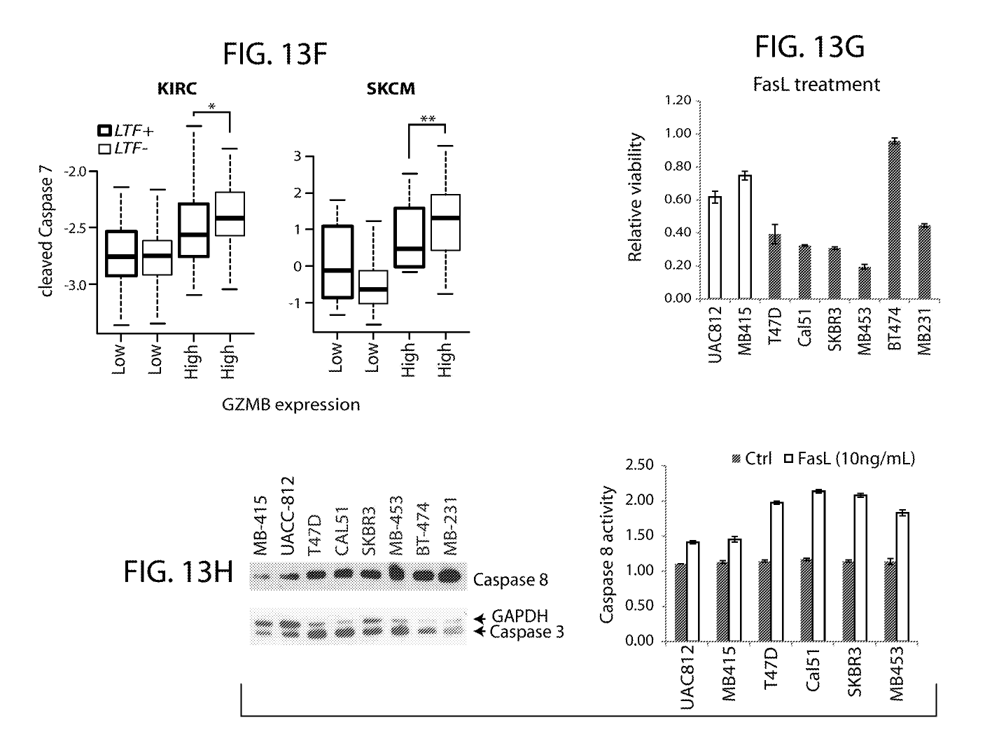

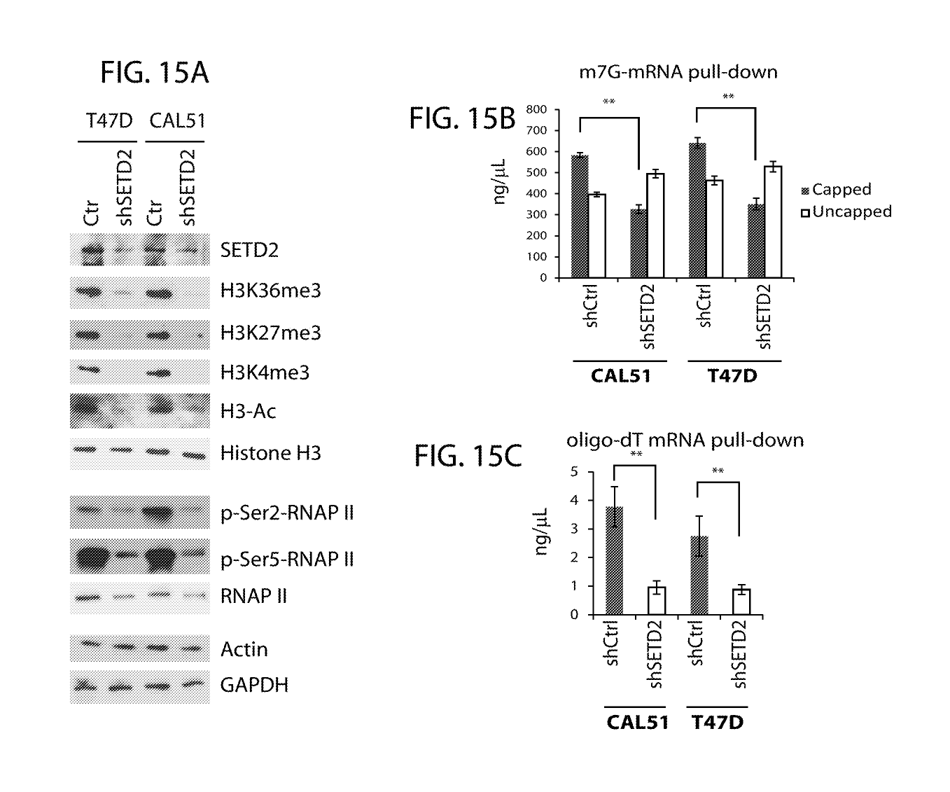

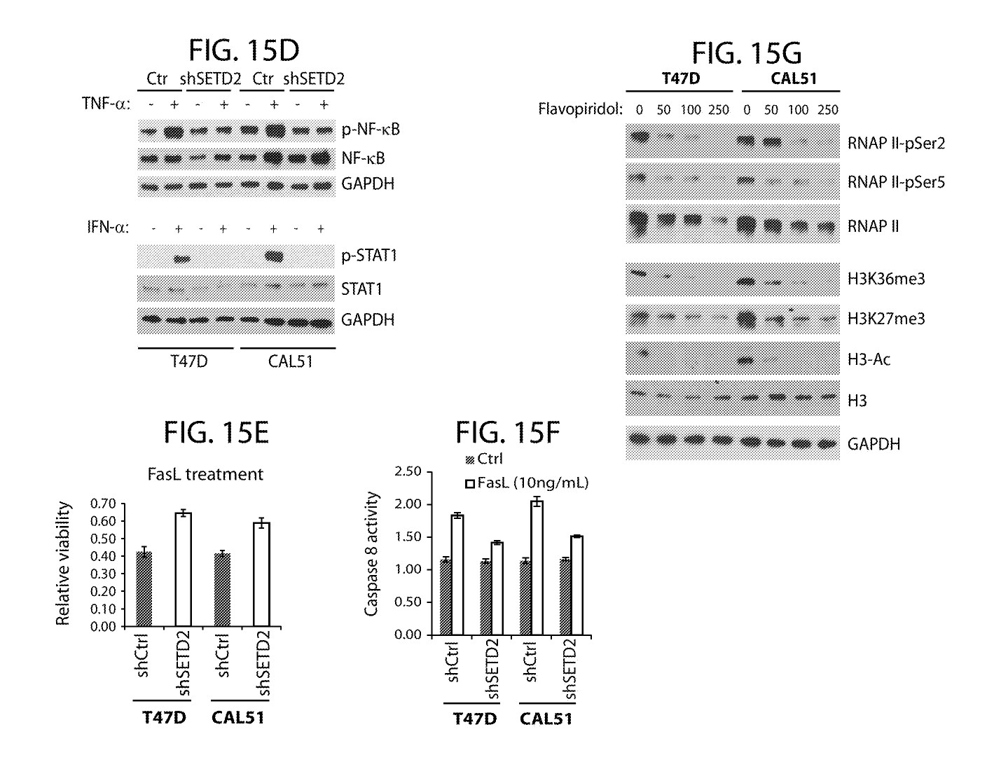

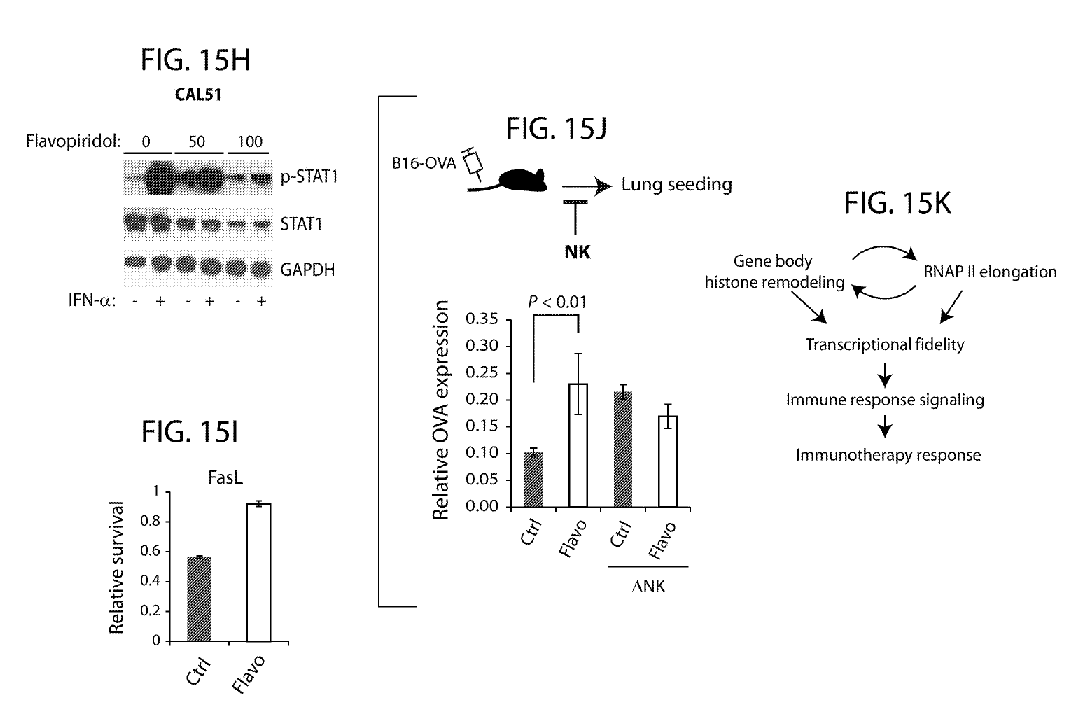

LTF is a previously uncharacterized phenotype that is observed more than 10% of all cancers, where defects in almost the entire epigenetic and transcriptional apparatus leads to a highly conserved molecular phenotype. Due to defective transcriptional elongation by RNA polymerase II (RNAP II), the transcription of long genes in the genome is impaired in LTF+ tumor cells. Interestingly, the inflammatory response pathways, including TNFα, Fas and interferon signaling, are mostly regulated by longer genes; and thus, their expression is severely reduced at both mRNA and protein levels (Example 5, FIG. 9). As such, LTF+ cells were defective in their response to pro-inflammatory cytotoxic stimuli, resisted anti-tumor innate responses in vivo, and correlated with worse prognosis in immunotherapy-, but not chemo- or targeted therapy-, treated patients (Examples 7 and 9, FIGS. 13 and 15). Therefore, widespread loss of epigenetic and transcriptional functions in tumors can impose a stable immune-ignorant state, which renders them resistant to tumor-priming inflammatory cytokines and anti-tumor immune attack mechanisms.

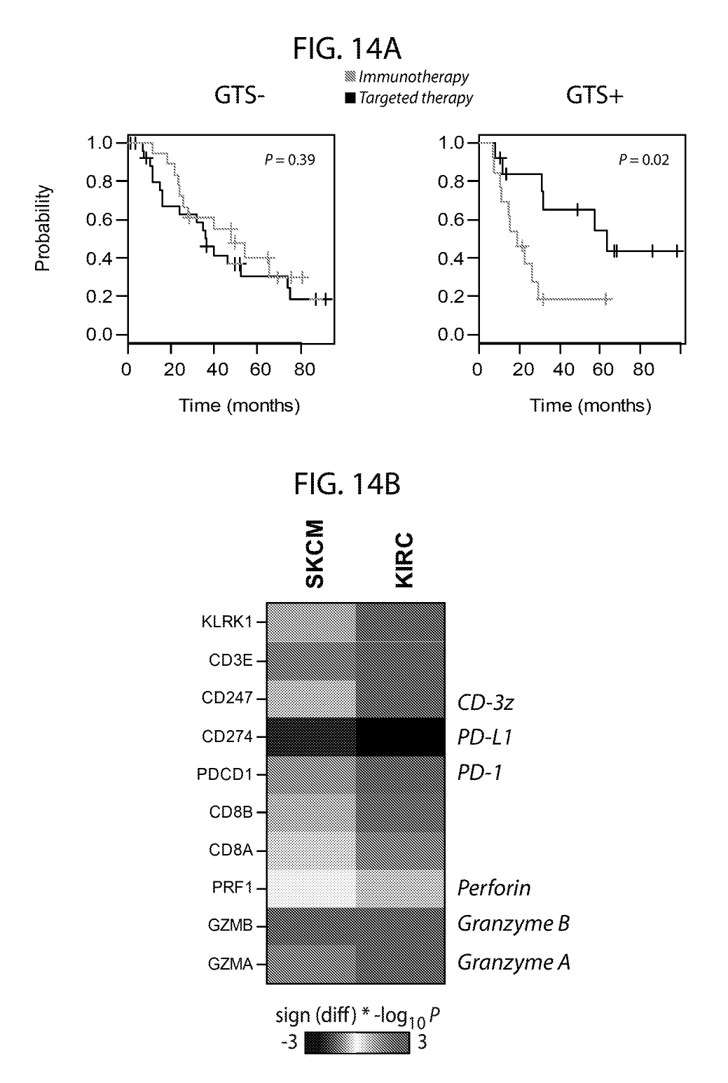

Mutations in genes involved in chromatin remodeling are common in clear cell renal cell carcinoma (KIRC) (Watson et al., 2013), but not as frequent in other adult cancers, especially in SETD2, whose nonsense mutations correlated with LTF in KIRC. As such, no strong correlates for LTF were found among somatic alterations, including mutations in other chromatin modifiers such as EP300, ARID1A and MLL, in other cancers. Therefore, the majority of LTF cases may not be genetically defined. Given the complex and widespread aberrations in LTF, and the highly inter-dependent nature of the epigenetic and transcriptional machineries, LTF can be induced by multiple, even combinations of, different initiating mechanisms, selected for a tumorigenic advantage. LTF can be an adaptive mechanism of tumor cells to evade the host anti-tumor response, similar to mutations in the initiator caspases 8 and 10 observed in high-tumor infiltration by lymphocytes (TIL) tumors. This is supported by the observation of higher immune cell infiltration in LTF+ tumors (see Example 13, FIG. 14B), possibly as a result of the immune response to genomic instability in these tumors, which in turn is an expected outcome of defective chromatin remodeling (Kanu et al., 2015; Pfister et al., 2014).

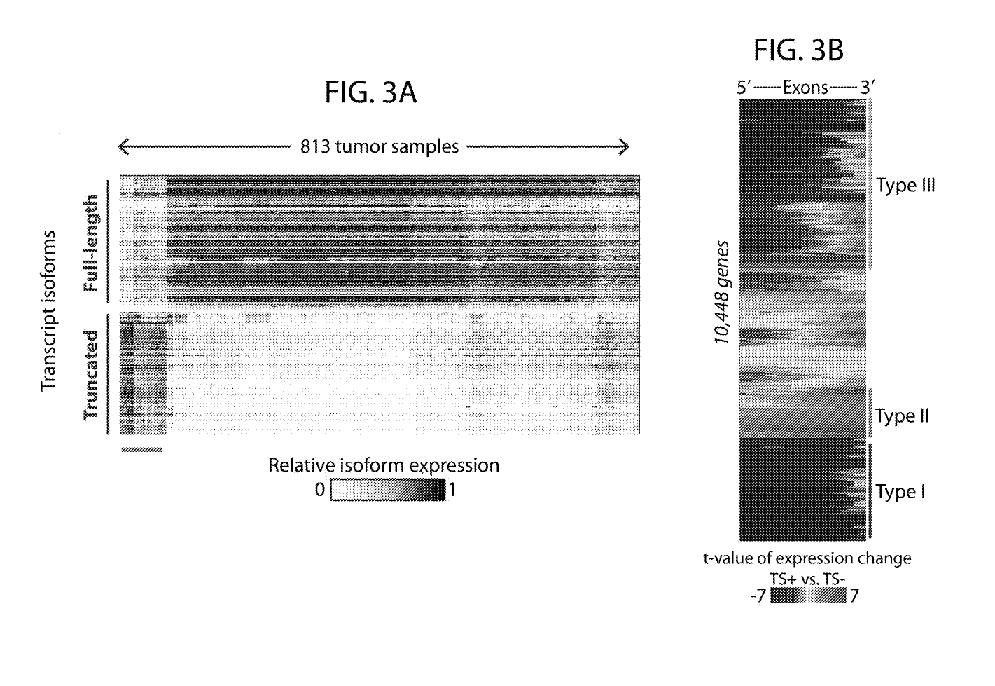

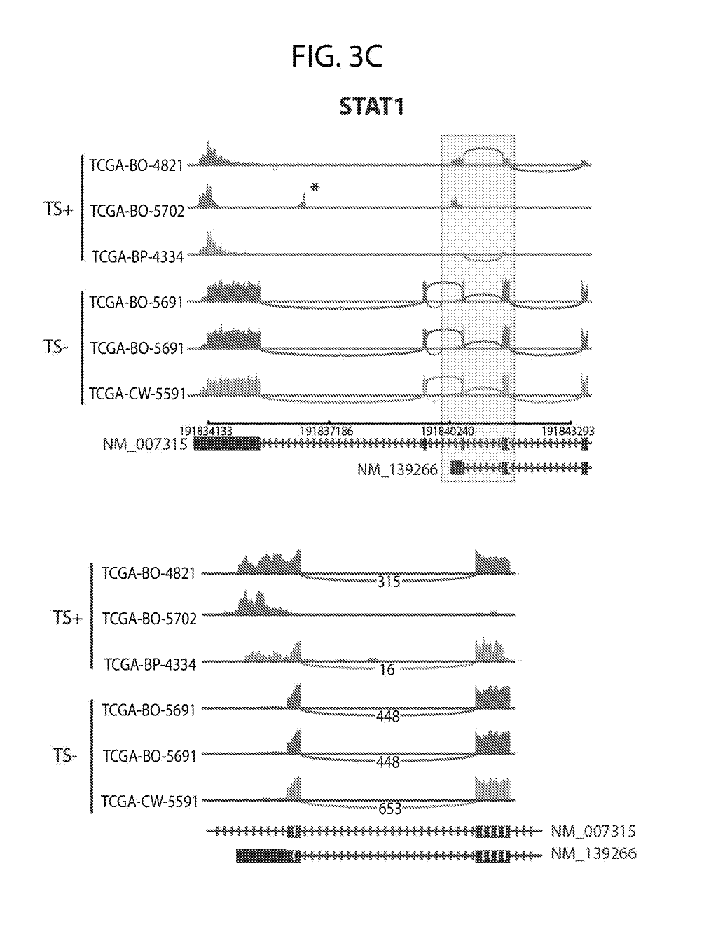

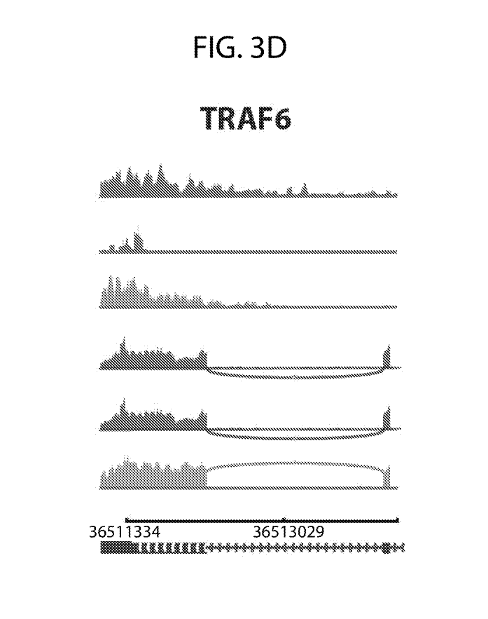

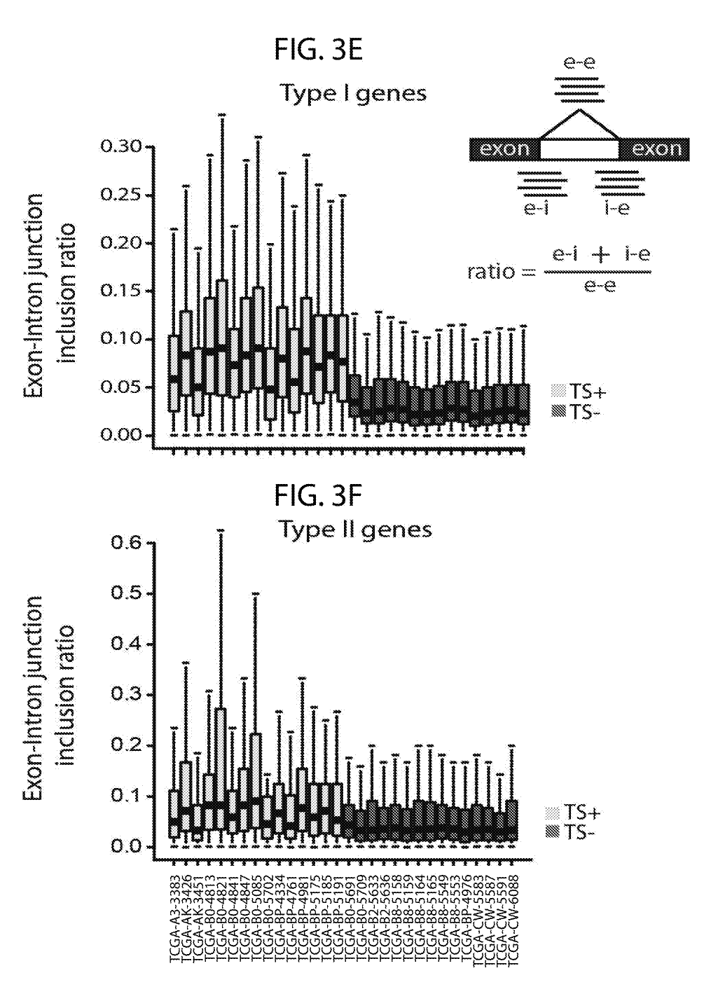

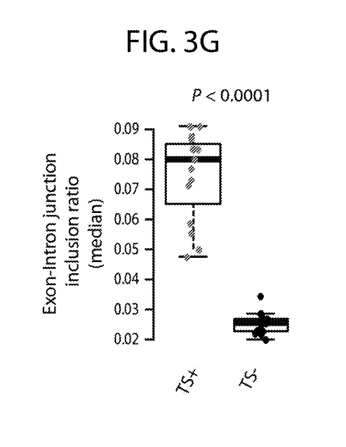

Loss of 5′exon expression in LTF is reminiscent of poly-A selection bias in the sequencing of degraded tissue RNA, indicating that LTF may be an artifact of poor RNA quality. However, cryptic expression of introns and defective splicing, as well as highly consistent non-RNA aberrations observed in LTF+ cancers, such as DNA methylation defects and protein-level signaling pathway changes that are consistent with mRNA expression changes, cannot be explained by tissue RNA degradation. Moreover, a highly similar phenotype was observed in cell lines, where many of the epigenetic and functional implications of the LTF phenotype observed in tissue samples were experimentally validated. However, the cryptic random transcription along the gene bodies, as predicted by this model of LTF, would falsely manifest in the observed loss of 5′ exon expression (see Example 2, FIG. 3B) upon poly-A mRNA selection, as only those rare mRNAs that were properly terminated at the 3′ end would be selected for sequencing. Therefore, detection of widespread 5′-shortening of transcripts has more value as a marker of LTF in poly-A-selected samples, rather than as a mechanistic view of mRNA transcription defects in LTF+ cells. Nevertheless, the computational and experimental analyses described herein demonstrate that the main molecularly and clinically significant phenotypes, including widespread cryptic transcription and immune resistance, are true biological phenomena. Knowledge of the mechanisms of induction of LTF and its sustenance in cancers can enable the design of therapeutic strategies to reverse it in cancer treatment, including in treatments involving chemotherapy and/or targeted therapy and/or alternative therapy, as well as in treatments involving immunotherapy. In addition, the specific vulnerabilities imposed by the LTF phenotype can be identified and exploited to have high translational value for cancer therapy, given that LTF is observed in a substantial portion of cancers.

In some embodiments, an LTF phenotype can be associated with a type of cancer, such as cancers of the skin, bone, breast, kidney, brain, head and neck, lung, ovary, uterus, cervix, blood, bladder, pancreas, liver, stomach, esophagus, prostate, colon, thyroid, and the like.

### Immunotherapy.

Immunotherapy, wherein a disease is treated by inducing, enhancing, or suppressing an immune response, is revolutionizing cancer care with a promise of cure for a select population of patients (Sharma and Allison, 2015). Unfortunately, there are no clear biomarkers to differentiate between potentially responding and non-responding patients. To date, infiltration of tumors by lymphocytes has been one of the strongest markers of later response, although many patients with high TIL do not respond (Tumeh et al., 2014; Van Allen et al., 2015b). Importantly, LTF predicted immunotherapy response independent of TIL, as LTF correlated with higher TIL expression in most cases, indicating that LTF can be a tumor-intrinsic mechanism of resistance to TIL-mediated anti-tumor attack. Accordingly, combining LTF and TIL status significantly improved the prognostic power in immunotherapy-treated patients (see Example 7, FIG. 13D). Therefore, LTF is an important tumor-intrinsic marker of immunotherapy response and can be used alone or in combination with the existing TIL-based markers for improved prediction of response.

In some embodiments, a subject having cancer or at least one symptom thereof can be treated based on the lack of suitability of immunotherapy where the tumor cells of the subject have an LTF phenotype, or based on the suitability of immunotherapy where the tumor cells of the subject lack an LTF phenotype. For example, a subject having an LTF phenotype can be administered or assigned a treatment which does not include immunotherapy, but does include one or more different forms of cancer therapy. For example, this includes chemotherapy, targeted therapy, alternative therapy, and the like. Conversely, a subject lacking an LTF phenotype can be administered or assigned a treatment which includes immunotherapy. The immunotherapy treatment can additionally include one or more different forms of cancer therapy. For example, this includes chemotherapy, targeted therapy, alternative therapy, and the like. In some embodiments, the treatment can be conducted as part of a clinical trial.

In some embodiments, immunotherapies include cell-based immunotherapies, such as those involving cells which effect an immune response (such as, for example, lymphocytes, macrophages, natural killer (NK) cells, dendritic cells, cytotoxic T lymphocytes (CTL), antibodies and antibody derivatives (such as, for example, monoclonal antibodies, conjugated monoclonal antibodies, polyclonal antibodies, antibody fragments, radiolabeled antibodies, chemolabeled antibodies, etc.), immune checkpoint inhibitors, vaccines (such as, for example, cancer vaccines (e.g. tumor cell vaccines, antigen vaccines, dendritic cell vaccines, vector-based vaccines, etc.), e.g. oncophage, sipuleucel-T, and the like), immunomodulators (such as, for example, interleukins, cytokines, chemokines, etc.), topical immunotherapies (such as, for example, imiquimod, and the like), injection immunotherapies, adoptive cell transfer, oncolytic virus therapies (such as, for example, talimogene laherparepvec (T-VEC), and the like), immunosuppressive drugs, helminthic therapies, other non-specific immunotherapies, and the like. Immune checkpoint inhibitor immunotherapies are those that target one or more specific proteins or receptors, such as PD-1, PD-L1, CTLA-4, and the like. Immune checkpoint inhibitor immunotherapies include ipilimumab (Yervoy), nivolumab (Opdivo), pembrolizumab (Keytruda), and the like. Non-specific immunotherpaies include cytokines, interleukins, interferons, and the like. In some embodiments, an immunotherapy assigned or administered to a subject can include an interleukin, and/or interferon (IFN), and/or one or more suitable antibody-based reagent, such as denileukin diftitox and/or administration of an antibody-based reagent selected from the group consisting of ado-trastuzumab emtansine, alemtuzumab, atezolizumab, bevacizumab, blinatumomab, brentuximab vedotin, cetuximab, catumaxomab, gemtuzumab, ibritumomab tiuxetan, ilipimumab, natalizumab, nimotuzumab, nivolumab, ofatumumab, panitumumab, pembrolizumab, rituximab, tositumomab, trastuzumab, vivatuxin, and the like. In some embodiments, an immunotherapy assigned or administered to a subject can include an indoleamine 2,3-dioxygenase (IDO) inhibitor, adoptive T-cell therapy, virotherapy (T-VEC), and/or any other immunotherapy whose efficacy extensively depends on anti-tumor immunity. Those skilled in the art can determine appropriate immunotherapy options, including treatments that have been approved and those that in clinical trials or otherwise under development.

In some embodiments, a subject having cancer or at least one symptom thereof can be stratified in a clinical trial based on whether the subject as an LTF phenotype. For example, a subject can be deemed unsuitable for immunotherapy where the tumor cells of the subject have an LTF phenotype, or a subject can be deemed suitable for immunotherapy where the tumor cells of the subject lack an LTF phenotype. Where a subject is deemed suitable for immunotherapy, the subject can be administered or assigned an immunotherapy treatment, alone or in combination with one or more different forms of cancer therapy.

### Chemotherapy/Targeted Therapy/Alternative Therapy

Cancers are commonly treated with chemotherapy and/or targeted therapy and/or alternative therapy. Chemotherapies act by indiscriminately targeting rapidly dividing cells, including healthy cells as well as tumor cells, whereas targeted cancer therapies rather act by interfering with specific molecules, or molecular targets, which are involved in cancer growth and progression. Targeted therapy generally targets cancer cells exclusively, having minimal damage to normal cells. Chemotherapies and targeted therapies which are approved and/or in the clinical trial stage are known to those skilled in the art. Any such compound can be utilized in the practice of the present invention.

For example, approved chemotherapies include abitrexate (Methotrexate Injection), abraxane (Paclitaxel Injection), adcetris (Brentuximab Vedotin Injection), adriamycin (Doxorubicin), adrucil Injection (5-FU (fluorouracil)), afinitor (Everolimus), afinitor Disperz (Everolimus), alimta (PEMETREXED), alkeran Injection (Melphalan Injection), alkeran Tablets (Melphalan), aredia (Pamidronate), arimidex (Anastrozole), aromasin (Exemestane), arranon (Nelarabine), arzerra (Ofatumumab Injection), avastin (Bevacizumab), beleodaq (Belinostat Injection), bexxar (Tositumomab), BiCNU (Carmustine), blenoxane (Bleomycin), blincyto (Blinatumoma b Injection), bosulif (Bosutinib), busulfex Injection (Busulfan Injection), campath (Alemtuzumab), camptosar (Irinotecan), caprelsa (Vandetanib), casodex (Bicalutamide), CeeNU (Lomustine), CeeNU Dose Pack (Lomustine), cerubidine (Daunorubicin), clolar (Clofarabine Injection), cometriq (Cabozantinib), cosmegen (Dactinomycin), cotellic (Cobimetinib), cyramza (Ramucirumab Injection), cytosarU (Cytarabine), cytoxan (Cytoxan), cytoxan Injection (Cyclophosphamide Injection), dacogen (Decitabine), daunoXome (Daunorubicin Lipid Complex Injection), decadron (Dexamethasone), depoCyt (Cytarabine Lipid Complex Injection), dexamethasone Intensol (Dexamethasone), dexpak Taperpak (Dexamethasone), docefrez (Docetaxel), doxil (Doxorubicin Lipid Complex Injection), droxia (Hydroxyurea), DTIC (Decarbazine), eligard (Leuprolide), ellence (Ellence (epirubicin)), eloxatin (Eloxatin (oxaliplatin)), elspar (Asparaginase), emcyt (Estramustine), erbitux (Cetuximab), erivedge (Vismodegib), erwinaze (Asparaginase Erwinia chrysanthemi), ethyol (Amifostine), etopophos (Etoposide Injection), eulexin (Flutamide), fareston (Toremifene), farydak (Panobinostat), faslodex (Fulvestrant), femara (Letrozole), firmagon (Degarelix Injection), fludara (Fludarabine), folex (Methotrexate Injection), folotyn (Pralatrexate Injection), FUDR (FUDR (floxuridine)), gazyva (Obinutuzumab Injection), gemzar (Gemcitabine), gilotrif (Afatinib), gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Ifex (Ifosfamide), Imbruvica (Ibrutinib), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Keytruda (Pembrolizumab Injection), Kyprolis (Carfilzomib), Lanvima (Lenvatinib), Leukeran (Chlorambucil), Leukine (Sargramostim), Leustatin (Cladribine), Lonsurf (Trifluridine and Tipiracil), Lupron (Leuprolide), Lupron Depot (Leuprolide), Lupron DepotPED (Leuprolide), Lynparza (Olaparib), Lysodren (Mitotane), Marquibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Odomzo (Sonidegib), Oncaspar (Pegaspargase), Oncovin (Vincristine), Ontak (Denileukin Diftitox), onxol (Paclitaxel Injection), opdivo (Nivolumab Injection), panretin (Alitretinoin), paraplatin (Carboplatin), perj eta (Pertuzumab Injection), platinol (Cisplatin), platinol (Cisplatin Injection), platinolAQ (Cisplatin), platinolAQ (Cisplatin Injection), pomalyst (Pomalidomide), prednisone Intensol (Prednisone), proleukin (Aldesleukin), purinethol (Mercaptopurine), reclast (Zoledronic acid), revlimid (Lenalidomide), rheumatrex (Methotrexate), rituxan (Rituximab), roferonA alfaa (Interferon alfa-2a), rubex (Doxorubicin), sandostatin (Octreotide), sandostatin LAR Depot (Octreotide), soltamox (Tamoxifen), sprycel (Dasatinib), sterapred (Prednisone), sterapred DS (Prednisone), stivarga (Regorafenib), supprelin LA (Histrelin Implant), sutent (Sunitinib), sylatron (Peginterferon Alfa-2b Injection (Sylatron)), sylvant (Siltuximab Injection), synribo (Omacetaxine Injection), tabloid (Thioguanine), taflinar (Dabrafenib), tarceva (Erlotinib), targretin Capsules (Bexarotene), tasigna (Decarbazine), taxol (Paclitaxel Injection), taxotere (Docetaxel), temodar (Temozolomide), temodar (Temozolomide Injection), tepadina (Thiotepa), thalomid (Thalidomide), theraCys BCG (BCG), thioplex (Thiotepa), TICE BCG (BCG), toposar (Etoposide Injection), torisel (Temsirolimus), treanda (Bendamustine hydrochloride), trelstar (Triptorelin Injection), trexall (Methotrexate), trisenox (Arsenic trioxide), tykerb (lapatinib), unituxin (Dinutuximab Injection), valstar (Valrubicin Intravesical), vantas (Histrelin Implant), vectibix (Panitumumab), velban (Vinblastine), velcade (Bortezomib), vepesid (Etoposide), vepesid (Etoposide Injection), vesanoid (Tretinoin), vidaza (Azacitidine), vincasar PFS (Vincristine), vincrex (Vincristine), votrient (Pazopanib), vumon (Teniposide), wellcovorin IV (Leucovorin Injection), xalkori (Crizotinib), xeloda (Capecitabine), xtandi (Enzalutamide), yervoy (Ipilimumab Injection), yondelis (Trabectedin Injection), zaltrap (Ziv-aflibercept Injection), zanosar (Streptozocin), zelboraf (Vemurafenib), zevalin (Ibritumomab Tiuxetan), zoladex (Goserelin), zolinza (Vorinostat), zometa (Zoledronic acid), zortress (Everolimus), zydelig (Idelalisib), zykadia (Ceritinib), zytiga (Abiraterone), and the like, in addition to analogs and derivatives thereof. For example, approved targeted therapies include ado-trastuzumab emtansine (Kadcyla), afatinib (Gilotrif), aldesleukin (Proleukin), alectinib (Alecensa), alemtuzumab (Campath), axitinib (Inlyta), belimumab (Benlysta), belinostat (Beleodaq), bevacizumab (Avastin), bortezomib (Velcade), bosutinib (Bosulif), brentuximab vedotin (Adcetris), cabozantinib (Cabometyx [tablet], Cometriq [capsule]), canakinumab (Ilaris), carfilzomib (Kyprolis), ceritinib (Zykadia), cetuximab (Erbitux), cobimetinib (Cotellic), crizotinib (Xalkori), dabrafenib (Tafinlar), daratumumab (Darzalex), dasatinib (Sprycel), denosumab (Xgeva), dinutuximab (Unituxin), elotuzumab (Empliciti), erlotinib (Tarceva), everolimus (Afinitor), gefitinib (Iressa), ibritumomab tiuxetan (Zevalin), ibrutinib (Imbruvica), idelalisib (Zydelig), imatinib (Gleevec), ipilimumab (Yervoy), ixazomib (Ninlaro), lapatinib (Tykerb), lenvatinib (Lenvima), necitumumab (Portrazza), nilotinib (Tasigna), nivolumab (Opdivo), obinutuzumab (Gazyva), ofatumumab (Arzerra, HuMax-CD20), olaparib (Lynparza), osimertinib (Tagrisso), palbociclib (Ibrance), panitumumab (Vectibix), panobinostat (Farydak), pazopanib (Votrient), pembrolizumab (Keytruda), pertuzumab (Perj eta), ponatinib (Iclusig), ramucirumab (Cyramza), rapamycin, regorafenib (Stivarga), rituximab (Rituxan, Mabthera), romidepsin (Istodax), ruxolitinib (Jakafi), siltuximab (Sylvant), sipuleucel-T (Provenge), sirolimus, sonidegib (Odomzo), sorafenib (Nexavar), sunitinib, tamoxifen, temsirolimus (Torisel), tocilizumab (Actemra), tofacitinib (Xeljanz), tositumomab (Bexxar), trametinib (Mekinist), trastuzumab (Herceptin), vandetanib (Caprelsa), vemurafenib (Zelboraf), venetoclax (Venclexta), vismodegib (Erivedge), vorinostat (Zolinza), ziv-aflibercept (Zaltrap), and the like, in addition to analogs and derivatives thereof. Those skilled in the art can determine appropriate chemotherapy and/or targeted therapy and/or alternative therapy options, including treatments that have been approved and those that in clinical trials or otherwise under development.

In some embodiments, a subject having an LTF phenotype can be administered or assigned a treatment which does not include immunotherapy, but does include one or more different forms of cancer therapy, whereas a subject lacking an LTF phenotype can be administered or assigned a treatment which includes immunotherapy. The immunotherapy treatment can additionally include one or more different forms of cancer therapy. For example, a treatment which includes one or more different forms of cancer therapy can include chemotherapy, targeted therapy, alternative therapy, and the like. In some embodiments, the treatment can be conducted as part of a clinical trial.

Some targeted therapies are also immunotherapies. In embodiments of the present invention, immunotherapy is not suitable for a subject having an LTF phenotype. Therefore, in such subjects, a targeted therapy to be administered is not an immunotherapy.

### Other Cancer Treatments

In addition to immunotherapies, chemotherapies, and targeted therapies, cancer can additionally be treated by other strategies. These include surgery, radiation therapy, hormone therapy, stem cell transplant, precision medicine, and the like; such treatments and the compounds and compositions utilized therein are known to those skilled in the art. Any such treatment strategies can be utilized in the practice of the present invention.

Alternative treatment strategies have also been used with various types of cancers. Such treatment can be used alone or in combination with any other treatment modality. These include exercise, massage, relaxation techniques, yoga, acupuncture, aromatherapy, hypnosis, music therapy, dietary changes, nutritional and dietary supplements, and the like; such treatments are known to those skilled in the art. Any such treatment strategies can be utilized in the practice of the present invention.

### Administration

Particular aspects of the invention relate to the use of cancer treatments, in the form of compounds and/or compositions, directly administered to a subject. Such compounds and/or compositions and/or their physiologically acceptable salts or esters, for the preparation of a medicament (pharmaceutical preparation). They can be converted into a suitable dosage form together with at least one solid, liquid and/or semiliquid excipient or assistant and, if desired, in combination with one or more further active ingredients.

Particular aspects of the invention furthermore include medicaments comprising at least one therapeutic compound or composition suitable for treatment of cancer, and/or its pharmaceutically usable derivatives, solvates and stereoisomers, including mixtures thereof in all ratios, and optionally excipients and/or assistants.

According to particular aspects, the therapeutic compounds and compositions can be administered by any conventional method available for use in conjunction with pharmaceutical drugs, either as individual therapeutic agents or in a combination of therapeutic agents. Such therapeutics can be administered by any pharmaceutically acceptable carrier, including, for example, any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional medium or agent is incompatible with the active compound, such media can be used in the compositions of the invention. Supplementary active compounds can also be incorporated into the compositions. A pharmaceutical composition in particular aspects of the invention is formulated to be compatible with its intended route of administration. Routes of administration include for example, but are not limited to, intravenous, intramuscular, and oral, and the like. Additional routes of administration include, for example, sublingual, buccal, parenteral (including, for example, subcutaneous, intramuscular, intraarterial, intradermal, intraperitoneal, intracisternal, intravesical, intrathecal, or intravenous), transdermal, oral, transmucosal, and rectal administration, and the like.

Solutions or suspensions used for appropriate routes of administration, including, for example, but not limited to parenteral, intradermal, or subcutaneous application, and the like, can include, for example, the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates, or phosphates and agents for the adjustment of tonicity such as sodium chloride or dextrose, and the like. The pH can be adjusted with acids or bases, such as, for example, hydrochloric acid or sodium hydroxide, and the like. The parenteral preparation can be enclosed in, for example, ampules, disposable syringes, or multiple dose vials made of glass or plastic, and the like.

Exemplary pharmaceutical compositions suitable for injectable use include, for example, sterile aqueous solutions (where water soluble) or dispersions and sterile powders for the extemporaneous preparation of sterile injectable solutions or dispersion, and the like. For intravenous administration, suitable carriers include, for example, physiological saline, bacteriostatic water, Cremophor ELTM (BASF, Parsippany, N.J.) or phosphate buffered saline (PBS), and the like. In all cases, the composition should be fluid to the extent that easy syringability exists. The carrier can be a solvent or dispersion medium containing, for example, water, ethanol, polyol (for example, glycerol, propylene glycol, and liquid polyethylene glycol, and the like), and suitable mixtures thereof, and the like. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, such as, for example, parabens, chlorobutanol, phenol, ascorbic acid, thimerosal, and the like. In many cases, it can be preferable to include isotonic agents, such as, for example, sugars, polyalcohols such as mannitol, sorbitol, and sodium chloride, and the like, in the composition. Prolonged absorption of the injectable compositions can be brought about by including in the composition an agent which delays absorption such as, for example, aluminum monostearate and gelatin, and the like.

Exemplary sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Exemplary oral compositions generally include an inert diluent or an edible carrier. They can be enclosed in gelatin capsules or compressed into tablets, for example. For oral administration, the agent can be contained in enteric forms to survive the stomach or further coated or mixed to be released in a particular region of the gastrointestinal (GI) tract by known methods. For the purpose of oral therapeutic administration, the active compound can be incorporated with excipients and used in the form of tablets, troches, or capsules, or the like. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following exemplary ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring, or the like. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatin, carbohydrates, such as lactose or starch, magnesium stearate, talc or VASELINE®. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders or also as nasal sprays. The novel compounds may also be lyophilized and the resultant lyophilizates used, for example, to prepare injection preparations. The preparations indicated may be sterilized and/or comprise assistants, such as lubricants, preservatives, stabilizers and/or wetting agents, emulsifying agents, salts for modifying the osmotic pressure, buffer substances, colorants and flavors and/or a plurality of further active ingredients, for example one or more vitamins.

For administration by inhalation, the compositions can be delivered in the form of an aerosol spray from pressured container or dispenser, which contains a suitable propellant, e.g., a gas such as carbon dioxide, or a nebulizer, or the like. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives, and the like. Transmucosal administration can be accomplished through the use of nasal sprays or suppositories. For transdermal administration, the active compounds are formulated into ointments, salves, gels, or creams as generally known in the art.

The compositions can also be prepared in the form of suppositories (e.g., with conventional suppository bases such as cocoa butter and other glycerides) or retention enemas for rectal delivery.

In particular embodiments, therapeutic compounds and/or compositions are prepared with carriers that will protect the compound against rapid elimination from the body, such as a controlled release formulation, including implants and microencapsulated delivery systems, and the like. Biodegradable, biocompatible polymers can be used, such as, for example, ethylene vinyl acetate, polyanhydrides, polyglycolic acid, collagen, polyorthoesters, and polylactic acid, and the like. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, which is incorporated herein by reference in its entirety.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The details for the dosage unit forms of the invention are dictated by and directly dependent on the unique characteristics of the active compound and the particular therapeutic effect to be achieved, and the limitations inherent in the art of compounding such an active compound for the treatment of individuals. Such details are known to those of skill in the art.

The dosage administered will, of course, vary depending upon known factors, such as the pharmacodynamic characteristics of the particular agent and its mode and route of administration; the age, health, sex, weight, and diet of the recipient; the nature and extent of the symptoms; the kind of concurrent treatment; the time and frequency of treatment; the excretion rate; and the effect desired. A daily dosage of active ingredient can be expected to be about 0.001 to 1000 milligrams (mg) per kilogram (kg) of body weight, with the preferred dose being 0.01 to about 30 mg/kg.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Expression of Truncated mRNA Isoforms in Cancers

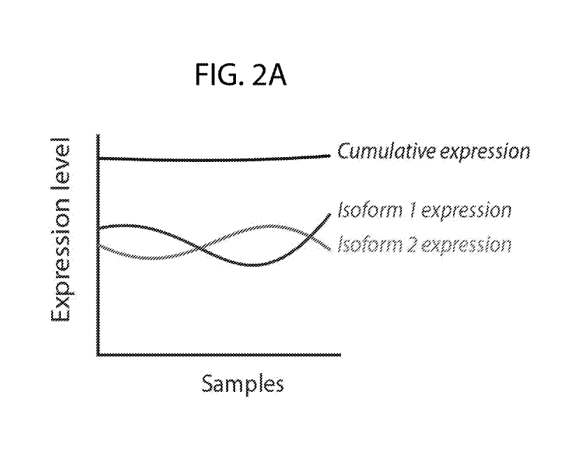

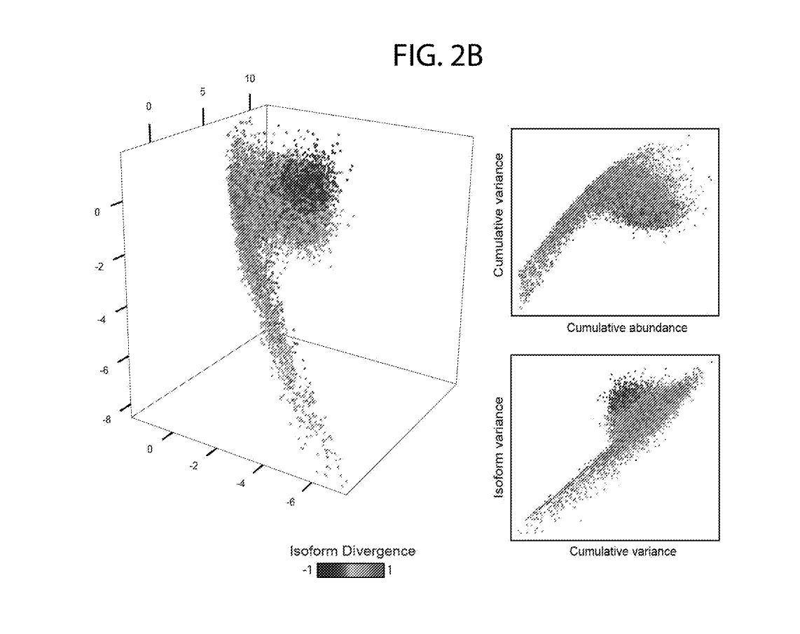

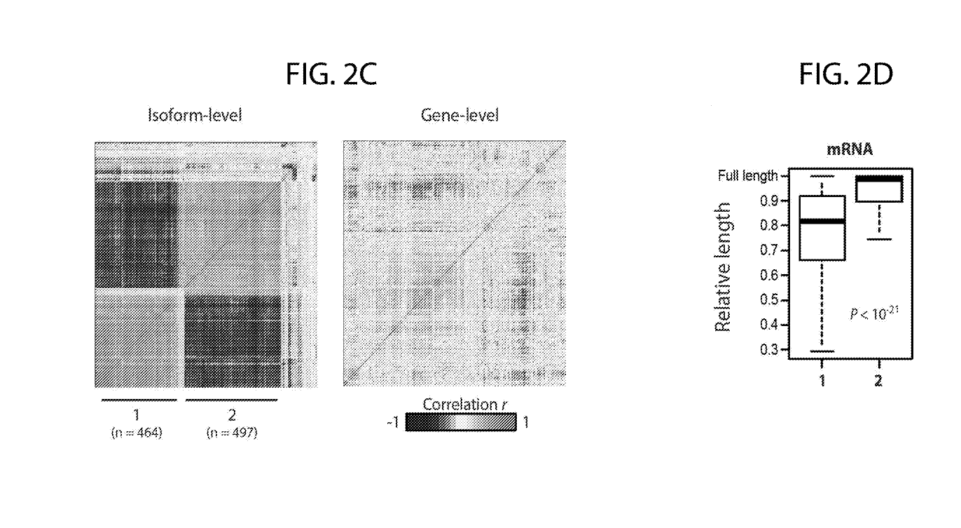

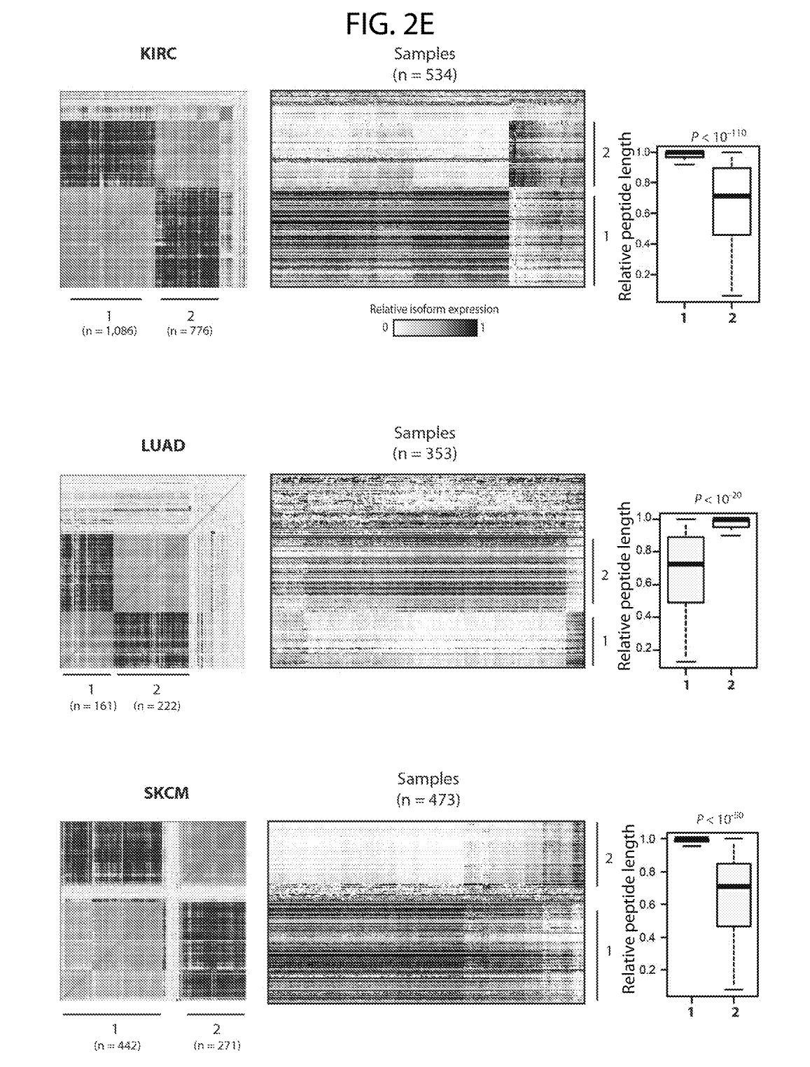

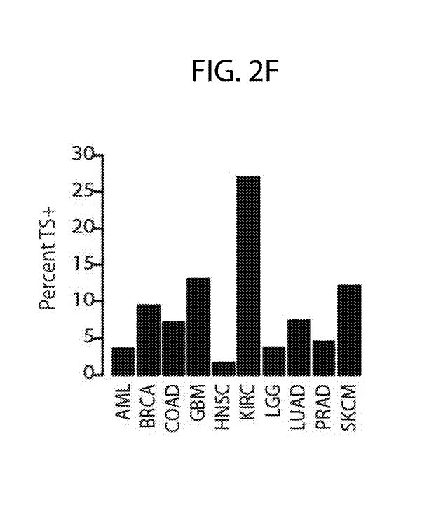

To gain insight into patterns of global transcriptional aberrations, the transcript isoform expression quantitation data from TCGA datasets were used to determine if there are aberrant patterns of alternative transcript expression in cancers, which could potentially indicate widespread transcriptional defects. Four gene-level metrics were defined (FIG. 2A): 1) cumulative expression (CE) as the sum of individual isoform expression levels for a gene in a given sample, 2) cumulative abundance (CA) as a measure of the average gene CE across samples, 3) cumulative variance (CV) as the variance in the CE, 4) isoform variance (IV) as the variance in the expression of an individual mRNA isoform, and 4) isoform divergence (ID) as the most negative correlation (Pearson's r) between the expressions of mRNA isoforms for a given gene. A strong negative ID (e.g. <−0.5) indicates that at least two isoforms of a gene have a mutually exclusive expression pattern, and hence, implies that the gene is at least partially regulated at the level of isoform switching where the expression of one mRNA isoform is substituted by another. A 3-dimensional plot relating these measures to each other for all genes expressed in breast cancer samples is shown in FIG. 2B.

Next, it was determined whether alternate transcripts of genes were co-regulated in trans; that is, if mRNA isoforms of a gene were differentially co-regulated with mRNA isoforms of another, perhaps reflecting a coordinate alternative transcript expression program. To test this, all pair-wise expression correlations of mRNA isoforms of genes that had ID values of lower than −0.2 in breast cancers (n=1,146 transcripts from 696 genes) were calculated. Strikingly, most mRNA transcripts clustered into two highly negatively correlated (i.e. mutually exclusively expressed) groups, which was not observed at the level of gene expression (FIG. 2C). Intriguingly, while one of the isoform groups largely represented the mRNA isoforms that coded for full-length proteins, the other group was almost exclusively characterized by mRNAs predicted to code for shorter truncated proteins (FIG. 2D).

The pattern of bimodal distribution of expressions of short and long isoforms for genes with negative ID values was observed in every cancer that was analyzed (FIG. 2E-F).

### Example 2

### Some Cancers Display Widespread Loss of Transcriptional Fidelity

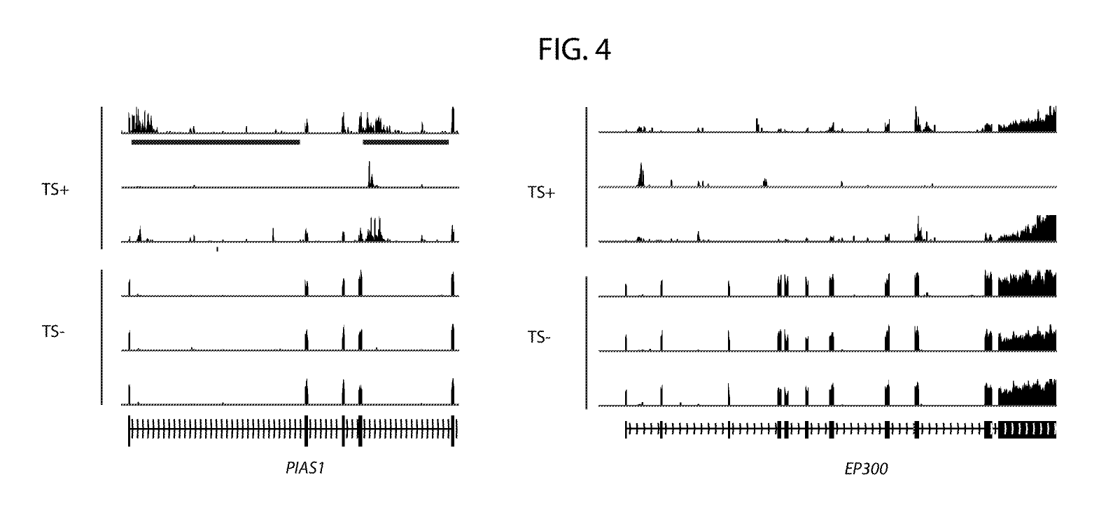

Through extensive pan-cancer analyses of isoform-specific mRNA expression patterns, a subset of almost every cancer type was found to preferentially express shorter truncated (aberrant or non-canonical) mRNA isoforms (see Example 1, FIG. 3A), indicating defective mRNA transcription or processing in these samples (transcript shortening: TS). To gain deeper insight into the transcriptional aberrations in these tumors, an analysis of differential expression in these tumors at the level of exons was performed. To enable a visual intuitive analysis of differential exon expression events in a matrix heatmap, every gene was binned into 20 exon bins, and a heatmap matrix was constructed, showing relative expression of the exon bins for each of the genes in tumors with TS. Remarkably, the exon t-value heatmap of the 10,448 genes that were expressed (i.e. 90%-ile expression level >30 normalized counts) in clear cell renal carcinomas (KIRC) shows that almost two thirds of all measured genes had a widespread significant loss in the expressions of their 5′ exons to variable degrees, while still many were significantly overexpressed in tumors with TS (FIG. 3B, see also Table 1, listing Type I and Type II genes; Type III genes not listed). Furthermore, a visual analysis of read mappings along genes indicated that Type I genes (see FIG. 3B), in addition to reduced 5′ exon expression levels, also frequently had markedly increased presence of intronic reads, premature transcription termination and poor exon definition (FIGS. 3C-D and FIG. 4).

TABLE 1
Type I and Type II genes (official gene names
from HUGO Gene Nomenclature Committee (HGNC)).

| Type I | Type II |
|---|---|
| AASDHPPT | AAAS |
| ABCA1 | AARSD1 |
| ABCB10 | ABCB8 |
| ABCB7 | ABCC10 |
| ABCC1 | ABHD11 |
| ABCC4 | ABHD14A |
| ABCD3 | ACAP1 |
| ABCE1 | ACBD4 |
| ABCF1 | ACCS |
| ABHD2 | ACOT8 |
| ABI1 | ACTR5 |
| ABI2 | ADRM1 |
| ABL1 | AGPAT2 |
| ABLIM1 | AIFM2 |
| ACADM | AIP |
| ACADSB | AKR1A1 |
| ACAP2 | ALG3 |
| ACBD3 | ALKBH2 |
| ACBD5 | ALKBH6 |
| ACLY | ALKBH7 |
| ACOX1 | ANAPC11 |
| ACSL3 | ANKRD13D |
| ACSL4 | ANKRD37 |
| ACTR2 | ANKRD39 |
| ACTR3 | ANKRD54 |
| ACVR1 | ANKS3 |
| ACVR1B | AP2S1 |
| ADAM10 | AP4M1 |
| ADAM17 | APBA3 |
| ADAM9 | APBB3 |
| ADAMTSL3 | APOA1BP |
| ADAR | APOO |
| ADARB1 | APRT |
| ADAT1 | ARF5 |
| ADCY6 | ARL2 |
| ADCY9 | ARL6IP4 |
| ADD1 | ARMC6 |
| ADD3 | ARPC3 |
| ADIPOR1 | ARRDC1 |
| ADIPOR2 | ASPSCR1 |
| ADNP | ATAD3B |
| ADPGK | ATG4D |
| ADSS | ATOX1 |
| AEBP2 | ATP5D |
| AFAP1 | ATP5E |
| AFF1 | ATP5G1 |
| AFF4 | ATP5G2 |
| AFTPH | ATP5H |
| AGAP1 | ATP5I |
| AGFG1 | ATP5J2 |
| AGGF1 | ATP5O |
| AGL | ATP6V0C |
| AGPAT1 | ATP6V1G1 |
| AGPAT5 | ATPIF1 |
| AGPS | AUP1 |
| AGRN | AURKAIP1 |
| AHCTF1 | AXIN2 |
| AHCYL1 | B3GAT3 |
| AHR | B4GALT7 |
| AIDA | B9D2 |
| AIM1 | BAD |
| AKAP10 | BANF1 |
| AKAP11 | BAX |
| AKAP13 | BCAP31 |
| AKIRIN1 | BCAT2 |
| AKT1 | BCL2L12 |
| AKT3 | BCL7C |
| ALAD | BCS1L |
| ALCAM | BGLAP |
| ALDH5A1 | BID |
| ALDH9A1 | BLOC1S1 |
| ALG9 | BLVRB |
| ALKBH5 | BOLA3 |
| AMD1 | BRMS1 |
| AMOT | BSCL2 |
| AMOTL1 | BSG |
| ANAPC1 | BUD31 |
| ANK3 | C10orf11 |
| ANKFY1 | C11orf1 |
| ANKIB1 | C11orf10 |
| ANKLE2 | C11orf31 |
| ANKMY2 | C11orf48 |
| ANKRD12 | C11orf51 |
| ANKRD13A | C11orf59 |
| ANKRD13C | C11orf67 |
| ANKRD17 | C11orf73 |
| ANKRD27 | C11orf74 |
| ANKRD28 | C11orf83 |
| ANKRD40 | C12orf10 |
| ANKRD46 | C12orf45 |
| ANKRD52 | C12orf57 |
| ANKRD6 | C12orf62 |
| ANO6 | C13orf27 |
| ANP32E | C14orf153 |
| ANTXR2 | C14orf156 |
| AP1AR | C14orf179 |
| AP1B1 | C14orf2 |
| AP1G1 | C15orf63 |
| AP2A2 | C16orf13 |
| AP2B1 | C16orf42 |
| AP3B1 | C16orf48 |
| AP3D1 | C16orf53 |
| AP3M1 | C16orf61 |
| AP3M2 | C16orf68 |
| APBB2 | C17orf106 |
| APH1A | C17orf37 |
| API5 | C17orf49 |
| APOL6 | C17orf61 |
| APP | C17orf70 |
| APPBP2 | C17orf79 |
| APPL1 | C17orf81 |
| APPL2 | C17orf89 |
| AQR | C18orf21 |
| ARCN1 | C19orf10 |
| ARFGAP2 | C19orf24 |
| ARFGAP3 | C19orf33 |
| ARFGEF1 | C19orf43 |
| ARFGEF2 | C19orf48 |
| ARFIP1 | C19orf53 |
| ARHGAP12 | C19orf56 |
| ARHGAP17 | C19orf60 |
| ARHGAP21 | C19orf66 |
| ARHGAP24 | C19orf70 |
| ARHGAP26 | C1orf151 |
| ARHGAP29 | C1orf31 |
| ARHGAP31 | C1orf35 |
| ARHGAP42 | C1orf54 |
| ARHGAP5 | C1orf66 |
| ARHGEF10 | C1orf86 |
| ARHGEF12 | C21orf57 |
| ARHGEF18 | C22orf32 |
| ARHGEF3 | C2orf28 |
| ARHGEF6 | C2orf7 |
| ARHGEF7 | C2orf76 |
| ARID1A | C2orf79 |
| ARID1B | C3orf75 |
| ARID2 | C4orf27 |
| ARID4A | C4orf47 |
| ARID4B | C6orf1 |
| ARID5B | C6orf108 |
| ARIH1 | C6orf125 |
| ARL5A | C6orf129 |
| ARMC1 | C6orf136 |
| ARMC8 | C6orf203 |
| ARMCX3 | C6orf26 |
| ARNT | C7orf11 |
| ARPP19 | C7orf47 |
| ARRDC3 | C7orf50 |
| ARRDC4 | C7orf55 |
| ARSD | C7orf59 |
| ASAP1 | C8orf30A |
| ASAP2 | C8orf38 |
| ASB8 | C8orf40 |
| ASCC1 | C8orf41 |
| ASCC3 | C8orf59 |
| ASH1L | C9orf100 |
| ASH2L | C9orf116 |
| ASPH | C9orf119 |
| ASXL1 | C9orf142 |
| ATAD1 | C9orf3 |
| ATE1 | C9orf89 |
| ATF1 | CAMTA1 |
| ATF2 | CAPG |
| ATF6 | CAPN12 |
| ATF7 | CARD16 |
| ATF7IP | CBR1 |
| ATG9A | CCDC101 |
| ATL3 | CCDC106 |
| ATMIN | CCDC107 |
| ATP10D | CCDC12 |
| ATP11A | CCDC124 |
| ATP11B | CCDC142 |
| ATP11C | CCDC159 |
| ATP13A3 | CCDC23 |
| ATP2A2 | CCDC24 |
| ATP2B1 | CCDC53 |
| ATP2B4 | CCDC56 |
| ATP2C1 | CCDC57 |
| ATP6V0A1 | CCDC58 |
| ATP6V1B2 | CCDC59 |
| ATP6V1C1 | CCDC61 |
| ATP8A1 | CCDC72 |
| ATP9A | CCDC84 |
| ATP9B | CCDC86 |
| ATRN | CCDC94 |
| ATRX | CCS |
| ATXN1L | CD320 |
| ATXN7L3 | CD63 |
| AVL9 | CDC34 |
| AXL | CDK2AP2 |
| AZIN1 | CDK5 |
| B3GALNT1 | CENPT |
| B4GALT1 | CEP290 |
| B4GALT5 | CHCHD1 |
| BACE1 | CHCHD5 |
| BACH1 | CHCHD8 |
| BAHD1 | CHEK2 |
| BAT2 | CHKB |
| BAT2L1 | CHMP2A |
| BAT2L2 | CHRNE |
| BAT3 | CIB1 |
| BAZ1B | CINP |
| BAZ2A | CISD3 |
| BAZ2B | CKS2 |
| BBS1 | CLPP |
| BBX | CLTA |
| BCAS3 | CLTB |
| BCL2L11 | CNPY2 |
| BCL7A | COASY |
| BCL9L | COMMD1 |
| BCLAF1 | COMMD4 |
| BCOR | COMMD6 |
| BDP1 | COMTD1 |
| BECN1 | COPE |
| BEND7 | COPS5 |
| BHLHE41 | COPS6 |
| BICC1 | COQ4 |
| BICD1 | CORO1B |
| BICD2 | COX17 |
| BIRC6 | COX4I1 |
| BIVM | COX5A |
| BLZF1 | COX5B |
| BMP2K | COX6A1 |
| BMPR2 | COX6B1 |
| BMS1 | COX6C |
| BNIP2 | COX7C |
| BPNT1 | COX8A |
| BPTF | CPSF3L |
| BRAF | CPT1B |
| BRCC3 | CREB3 |
| BRD2 | CRLS1 |
| BRD3 | CROCC |
| BRPF3 | CSNK2B |
| BSDC1 | CST3 |
| BTBD1 | CTDP1 |
| BTBD10 | CTU2 |
| BTBD3 | CUL9 |
| BZW1 | CUTA |
| C10orf119 | CWC15 |
| C10orf26 | CYB561D2 |
| C10orf46 | CYBA |
| C10orf72 | DAD1 |
| C10orf76 | DBI |
| C11orf54 | DCI |
| C12orf23 | DCXR |
| C13orf23 | DDAH2 |
| C14orf21 | DDT |
| C14orf43 | DDX39 |
| C16orf62 | DDX49 |
| C16orf70 | DDX56 |
| C16orf72 | DGCR6 |
| C17orf85 | DGCR6L |
| C18orf19 | DGUOK |
| C19orf2 | DHPS |
| C1orf106 | DHRS7B |
| C1orf107 | DHX34 |
| C1orf144 | DHX58 |
| C1orf25 | DMAP1 |
| C1orf27 | DMPK |
| C1orf55 | DNAJC17 |
| C1orf58 | DNAJC4 |
| C1orf9 | DNLZ |
| C1QTNF9 | DNTTIP1 |
| C20orf194 | DPM2 |
| C22orf13 | DPM3 |
| C3orf17 | DPP7 |
| C3orf63 | DPY30 |
| C3orf64 | DRAP1 |
| C4orf34 | DRG2 |
| C4orf41 | DTNB |
| C5orf22 | DTX2 |
| C5orf33 | DTYMK |
| C5orf4 | DUS1L |
| C5orf41 | DUS3L |
| C6orf106 | DUSP23 |
| C6orf192 | DYNLL1 |
| C6orf211 | DYNLRB1 |
| C6orf62 | DYNLT1 |
| C6orf89 | DYRK4 |
| C7orf42 | E4F1 |
| C8orf83 | EBP |
| C9orf129 | ECH1 |
| C9orf25 | EDF1 |
| C9orf5 | EEF1D |
| C9orf64 | EFHD2 |
| CAB39 | EGFL8 |
| CACNA2D1 | EIF3F |
| CADPS2 | EIF3G |
| CALCRL | EIF3I |
| CALD1 | EIF3K |
| CAMK2D | EIF4EBP1 |
| CAMK2G | EIF4EBP3 |
| CAMKK2 | EIF5A |
| CAMSAP1 | EIF6 |
| CAMSAP1L1 | ELOF1 |
| CAND1 | EMG1 |
| CANX | EMP3 |
| CAP1 | ENDOG |
| CAP2 | EPOR |
| CAPN2 | ERCC1 |
| CAPN7 | ERH |
| CAPRIN1 | ETFB |
| CAPZA1 | ETHE1 |
| CASC3 | EXOSC1 |
| CASC4 | EXOSC4 |
| CASD1 | EXOSC5 |
| CASP10 | EXOSC7 |
| CASP2 | F8A1 |
| CASP7 | FADS3 |
| CASP8 | FAHD2A |
| CAST | FAM100A |
| CBFB | FAM100B |
| CBLL1 | FAM108A1 |
| CBX6 | FAM113A |
| CCDC132 | FAM128A |
| CCDC25 | FAM128B |
| CCDC43 | FAM158A |
| CCDC47 | FAM162A |
| CCDC6 | FAM165B |
| CCDC69 | FAM173A |
| CCDC93 | FAM176B |
| CCND1 | FAM195A |
| CCNG2 | FAM195B |
| CCNT1 | FAM3A |
| CCNT2 | FAM50A |
| CCNY | FAM58A |
| CCNYL1 | FAM96B |
| CCPG1 | FAM98C |
| CD109 | FANCA |
| CD164 | FASTK |
| CD2AP | FAU |
| CD302 | FBF1 |
| CD4 | FBXL15 |
| CD46 | FBXL6 |
| CD84 | FBXW5 |
| CD99L2 | FDX1L |
| CDC14B | FER1L4 |
| CDC23 | FIBP |
| CDC27 | FKBP2 |
| CDC40 | FKBP8 |
| CDC42 | FLAD1 |
| CDC42BPA | FLJ35220 |
| CDC42BPB | FLYWCH2 |
| CDC42SE1 | FUT5 |
| CDC42SE2 | G6PC3 |
| CDC5L | GABARAP |
| CDC73 | GADD45GIP1 |
| CDH2 | GALK1 |
| CDH6 | GALT |
| CDK12 | GAMT |
| CDK13 | GCAT |
| CDK14 | GEMIN6 |
| CDK17 | GEMIN7 |
| CDK19 | GGTLC2 |
| CDKAL1 | GIYD2 |
| CECR1 | GLRX |
| CELF1 | GLRX2 |
| CELSR1 | GLTSCR2 |
| CEP120 | GMPPA |
| CEP57 | GNAS |
| CEP68 | GNB2L1 |
| CEPT1 | GNG5 |
| CERK | GNPTG |
| CHD1 | GPAT2 |
| CHD2 | GPATCH1 |
| CHD4 | GPR137 |
| CHD8 | GPR172A |
| CHD9 | GPS2 |
| CHM | GPX1 |
| CHMP7 | GPX4 |
| CHST9 | GSDMD |
| CHTF8 | GSTK1 |
| CHUK | GSTO1 |
| CKAP5 | GTF2E2 |
| CLASP1 | GTF2H5 |
| CLASP2 | GTF3A |
| CLCC1 | GTF3C6 |
| CLCN3 | GUK1 |
| CLCN5 | GZMA |
| CLDN12 | GZMH |
| CLIC4 | HAGH |
| CLINT1 | HAUS1 |
| CLIP1 | HAX1 |
| CLOCK | HBXIP |
| CLPX | HCFC1R1 |
| CLSTN1 | HCST |
| CLTC | HDDC3 |
| CMIP | HDHD3 |
| CMPK1 | HEATR7A |
| CNBP | HEBP2 |
| CNKSR3 | HEMK1 |
| CNN3 | HES4 |
| CNNM3 | HEXIM2 |
| CNOT1 | HHLA3 |
| CNOT2 | HIGD2A |
| CNOT4 | HINT2 |
| CNOT6 | HLA-F |
| CNOT6L | HMBS |
| CNOT7 | HMG20B |
| CNOT8 | HMOX2 |
| CNST | HN1 |
| COG2 | HOMER3 |
| COG3 | HRAS |
| COG5 | HSBP1 |
| COG6 | HSCB |
| COL4A3BP | HSD17B10 |
| COPA | HSPB1 |
| COPB1 | HSPB11 |
| COPS2 | HSPBP1 |
| CORO1C | HSPE1 |
| COX15 | HYI |
| CPA3 | ICAM3 |
| CPD | ICT1 |
| CPEB2 | IDUA |
| CPEB4 | IF127 |
| CPNE3 | IFI27L1 |
| CPNE8 | IFI27L2 |
| CPSF2 | IFI35 |
| CPSF6 | IFITM3 |
| CPT1A | IFRD2 |
| CPT2 | IFT20 |
| CRCP | IFT27 |
| CREB1 | IL18BP |
| CREB3L2 | IL32 |
| CREBBP | ILKAP |
| CREBL2 | IMMP1L |
| CREG1 | INPP5E |
| CRIM1 | IRF3 |
| CRK | IRF7 |
| CRKL | ISG15 |
| CRLF3 | ISG20 |
| CRNKL1 | ISOC2 |
| CRTC3 | ISYNA1 |
| CS | ITGAE |
| CSDE1 | ITGB3BP |
| CSE1L | ITPA |
| CSF1R | JOSD2 |
| CSGALNACT2 | JTB |
| CSNK1A1 | KAT2A |
| CSNK1G3 | KCTD13 |
| CSNK2A1 | KIAA1731 |
| CTBP2 | KIF12 |
| CTBS | KIFC2 |
| CTCF | KNTC1 |
| CTDSP2 | KRT18 |
| CTDSPL2 | KRT8 |
| CTNNA1 | KRTCAP2 |
| CTNNB1 | LENG1 |
| CTNND1 | LGALS1 |
| CTR9 | LIMD2 |
| CTSO | LIME1 |
| CTSS | LIN37 |
| CTTN | LOC100288778 |
| CTTNBP2NL | LOC150381 |
| CUBN | LOC150776 |
| CUL2 | LOC375190 |
| CUL3 | LOC388789 |
| CUL4A | LOC388796 |
| CUL4B | LOC440957 |
| CUL5 | LOC550643 |
| CWC22 | LOC728855 |
| CWF19L2 | LOC728875 |
| CXorf38 | LRRC23 |
| CXorf56 | LRRC45 |
| CYB5D1 | LRWD1 |
| CYB5R1 | LSM1 |
| CYBASC3 | LSM10 |
| CYBB | LSM3 |
| CYFIP1 | LSM4 |
| CYLD | LSM7 |
| CYP4V2 | LSMD1 |
| CYP7B1 | LST1 |
| CYTH1 | LTB |
| CYTH3 | LUC7L |
| CYTSA | LZTS2 |
| DAB2 | MACROD1 |
| DAPK1 | MAD2L2 |
| DARS | MAL1 |
| DARS2 | MAGOH |
| DAZAP2 | MAGOHB |
| DBT | MANF |
| DCAF10 | MAP2K2 |
| DCAF12 | MAPK7 |
| DCAF5 | 2-Mar |
| DCAF6 | MCTS1 |
| DCAF7 | MDP1 |
| DCAF8 | ME3 |
| DCTD | MEA1 |
| DCTN1 | MED11 |
| DCTN4 | MED25 |
| DCTN5 | MED27 |
| DCUN1D1 | METRN |
| DCUN1D4 | METTL1 |
| DDAH1 | METTL11A |
| DDB1 | METTL3 |
| DDHD2 | METTL5 |
| DDX1 | MFSD3 |
| DDX17 | MGMT |
| DDX18 | MGST2 |
| DDX19A | MGST3 |
| DDX21 | MIA |
| DDX23 | MICALL2 |
| DDX3X | MIF |
| DDX42 | MIIP |
| DDX46 | MITD1 |
| DDX50 | MLST8 |
| DDX58 | MORN2 |
| DDX6 | MPG |
| DDX60 | MPV17 |
| DEK | MPV17L2 |
| DENND1A | MRPL11 |
| DENND4C | MRPL12 |
| DENND5A | MRPL14 |
| DENR | MRPL16 |
| DGCR2 | MRPL17 |
| DHTKD1 | MRPL18 |
| DHX15 | MRPL2 |
| DHX36 | MRPL20 |
| DHX38 | MRPL21 |
| DHX40 | MRPL22 |
| DHX8 | MRPL23 |
| DHX9 | MRPL24 |
| DIAPH1 | MRPL27 |
| DICER1 | MRPL28 |
| DIDO1 | MRPL36 |
| DIP2B | MRPL38 |
| DIP2C | MRPL40 |
| DIS3 | MRPL41 |
| DIS3L | MRPL47 |
| DIXDC1 | MRPL48 |
| DLAT | MRPL51 |
| DLC1 | MRPL52 |
| DLG1 | MRPL53 |
| DMD | MRPL54 |
| DMXL1 | MRPL55 |
| DNAJA2 | MRPS11 |
| DNAJB14 | MRPS15 |
| DNAJC10 | MRPS21 |
| DNAJC11 | MRPS24 |
| DNAJC13 | MRPS26 |
| DNAJC16 | MRPS34 |
| DNAJC3 | MRPS36 |
| DNAJC5 | MSH5 |
| DNM1L | MSRB2 |
| DOCK1 | MTCP1NB |
| DOCK4 | MTERFD1 |
| DOCK7 | MTX1 |
| DOCK8 | MUTYH |
| DOCK9 | MXD3 |
| DPP4 | MYEOV2 |
| DPP8 | MYL12B |
| DPY19L1 | MYL5 |
| DPY19L4 | MYL6 |
| DPYD | MYL6B |
| DPYSL2 | N6AMT2 |
| DSC2 | NAA10 |
| DSCR3 | NANS |
| DSP | NAPRT1 |
| DST | NARFL |
| DTX3L | NCAPH2 |
| DUSP16 | NCRNA00116 |
| DUSP3 | NCRNA00152 |
| DYM | NDUFA1 |
| DYNC1H1 | NDUFA11 |
| DYNC1I2 | NDUFA12 |
| DYNC1LI2 | NDUFA13 |
| DYNLL2 | NDUFA2 |
| DYRK1A | NDUFA3 |
| DYSF | NDUFA7 |
| DYX1C1 | NDUFA8 |
| EAF1 | NDUFAF2 |
| EARS2 | NDUFAF3 |
| EDEM1 | NDUFB1 |
| EDEM3 | NDUFB10 |
| EDIL3 | NDUFB11 |
| EEA1 | NDUFB2 |
| EFNB2 | NDUFB4 |
| EFR3A | NDUFB6 |
| EFTUD1 | NDUFB7 |
| EFTUD2 | NDUFB8 |
| EGFR | NDUFB9 |
| EGLN1 | NDUFC1 |
| EHBP1 | NDUFC2 |
| EHHADH | NDUFS5 |
| EHMT1 | NDUFS6 |
| EI24 | NDUFS7 |
| EIF2AK1 | NDUFS8 |
| EIF2AK2 | NDUFV2 |
| EIF2AK3 | NEDD8 |
| EIF2AK4 | NENF |
| EIF2C1 | NEURL4 |
| EIF2S1 | NFKBIB |
| EIF2S3 | NHP2 |
| EIF3A | NHP2L1 |
| EIF3J | NIT2 |
| EIF4B | NME1-NME2 |
| EIF4E3 | NME1 |
| EIF4EBP2 | NME2 |
| EIF4ENIF1 | NME3 |
| EIF4G1 | NME4 |
| EIF4G2 | NMRAL1 |
| EIF4G3 | NOC4L |
| EIF4H | NOL12 |
| EIF5 | NOP10 |
| ELAVL1 | NOP16 |
| ELF1 | NOSIP |
| ELF2 | NOXA1 |
| ELF4 | NPR2 |
| ELK3 | NPRL2 |
| ELMOD2 | NR2C2AP |
| ELOVL5 | NR2F6 |
| ELP2 | NSUN5 |
| EML1 | NSUN5P1 |
| EML4 | NSUN5P2 |
| ENAH | NT5C |
| ENPEP | NUBP2 |
| ENPP4 | NUDC |
| EP300 | NUDT1 |
| EP400 | NUDT14 |
| EPAS1 | NUDT2 |
| EPB41L2 | NUDT22 |
| EPB41L4A | NUDT5 |
| EPC1 | NUDT8 |
| EPC2 | NUPR1 |
| EPHA7 | NUTF2 |
| EPN2 | OAF |
| EPRS | OAZ1 |
| EPS15 | OCEL1 |
| ERAP1 | ODF3B |
| ERBB2IP | ORMDL2 |
| ERC1 | OSGEP |
| ERGIC1 | OST4 |
| ERLIN1 | OTUB1 |
| ERLIN2 | P2RX5 |
| ERMP1 | P4HTM |
| ERO1LB | PABPC1L |
| ESYT1 | PAFAH1B3 |
| ESYT2 | PARK7 |
| ETF1 | PCBP4 |
| ETNK1 | PCGF1 |
| ETS1 | PCNT |
| ETV5 | PCYT2 |
| EVC | PDCD5 |
| EXD2 | PDE6D |
| EXOC1 | PDRG1 |
| EXOC2 | PDZD11 |
| EXOC4 | PEX16 |
| EXOC5 | PFDN2 |
| EXOC6 | PFDN4 |
| EXOC6B | PFDN5 |
| EZH1 | PFDN6 |
| F11R | PFN1 |
| FAF1 | PGLS |
| FAF2 | PHKG2 |
| FAM107B | PHLDA3 |
| FAM114A2 | PHPT1 |
| FAM115A | PIH1D1 |
| FAM116A | PIN1 |
| FAM120A | PIN4 |
| FAM120B | PIR |
| FAM122B | PKMYT1 |
| FAM129A | PL-5283 |
| FAM129B | PLA2G16 |
| FAM134C | PLEKHO1 |
| FAM13A | PLP2 |
| FAM13B | PMF1 |
| FAM149B1 | PNKP |
| FAM160B1 | POLD1 |
| FAM168A | POLD4 |
| FAM168B | POLE4 |
| FAM171A1 | POLG2 |
| FAM172A | POLR2F |
| FAM175B | POLR2G |
| FAM178A | POLR2H |
| FAM18B | POLR2I |
| FAM190B | POLR2J |
| FAM198B | POLR2L |
| FAM199X | POLRMT |
| FAM20B | POMP |
| FAM21C | POP5 |
| FAM35A | POP7 |
| FAM38A | PPAN-P2RY11 |
| FAM53C | PPAN |
| FAM59A | PPDPF |
| FAM73A | PPIA |
| FAM8A1 | PPIB |
| FAM91A1 | PPIH |
| FAM98A | PPOX |
| FAM98B | PPP1CA |
| FANCC | PPP1R14B |
| FAR1 | PPP1R16A |
| FAS | PPP4C |
| FAT1 | PPPDE2 |
| FBXL17 | PQBP1 |
| FBXL3 | PQLC2 |
| FBXL4 | PRDX1 |
| FBXL5 | PRDX5 |
| FBXO11 | PRELID1 |
| FBXO21 | PRICKLE4 |
| FBXO28 | PRKCDBP |
| FBXO3 | PRMT1 |
| FBXO38 | PRR5 |
| FBXW11 | PSENEN |
| FBXW2 | PSMA3 |
| FCF1 | PSMA4 |
| FCHO2 | PSMA7 |
| FCHSD2 | PSMB1 |
| FECH | PSMB10 |
| FERMT2 | PSMB3 |
| FEZ2 | PSMB4 |
| FGD4 | PSMB5 |
| FGFRL1 | PSMB6 |
| FGL2 | PSMB8 |
| FKBP15 | PSMB9 |
| FKBP9 | PSMC3 |
| FLI1 | PSMC3IP |
| FLNA | PSMC4 |
| FLOT2 | PSMC5 |
| FLT1 | PSMD13 |
| FMNL2 | PSMD6 |
| FMR1 | PSMD8 |
| FNBP1 | PSMD9 |
| FNBP1L | PSME1 |
| FNDC3A | PSME2 |
| FNDC3B | PSMG4 |
| FNIP1 | PTCD1 |
| FNIP2 | PTGES2 |
| FOXJ2 | PTOV1 |
| FOXJ3 | PTPRCAP |
| FOXN3 | PTRH1 |
| FPGT | PTTG1 |
| FPR3 | PUS1 |
| FRMD3 | PYCARD |
| FRMD4A | QTRT1 |
| FRMD4B | RAB24 |
| FRYL | RAB4B |
| FSTL1 | RABAC1 |
| FTO | RABEP2 |
| FTSJD2 | RABGGTA |
| FUBP1 | RAD9A |
| FUBP3 | RAG1AP1 |
| FUCA1 | RALY |
| FURIN | RANGRF |
| FXR2 | RARRES2 |
| FYCO1 | RARRES3 |
| FYTTD1 | RASSF7 |
| G3BP1 | RBM42 |
| G3BP2 | RBPMS |
| GAB1 | RBX1 |
| GAB2 | RDBP |
| GABPA | RDH5 |
| GALC | REXO1 |
| GALNT1 | RFNG |
| GALNT10 | RFXANK |
| GANAB | RGS14 |
| GAPVD1 | RHOC |
| GART | RHOD |
| GAS2L3 | RHPN1 |
| GATAD2A | RILP |
| GBE1 | RNASEH2C |
| GBF1 | RNASEK |
| GCC2 | RNASET2 |
| GCLC | RNF181 |
| GCLM | RNF25 |
| GCN1L1 | RNF7 |
| GDI2 | ROBLD3 |
| GEMIN5 | ROGDI |
| GFM1 | ROMO1 |
| GFPT1 | RP9 |
| GGA2 | RPAP1 |
| GGCX | RPL11 |
| GGNBP2 | RPL12 |
| GIGYF2 | RPL13 |
| GIT2 | RPL13A |
| GLE1 | RPL14 |
| GLG1 | RPL17 |
| GLUL | RPL18 |
| GLYR1 | RPL18A |
| GM2A | RPL19 |
| GMCL1 | RPL23 |
| GMFB | RPL23A |
| GNA12 | RPL24 |
| GNA13 | RPL26L1 |
| GNAI1 | RPL27 |
| GNAI3 | RPL27A |
| GNAQ | RPL28 |
| GNB1 | RPL29 |
| GNB4 | RPL3 |
| GNE | RPL30 |
| GNG12 | RPL32 |
| GNPDA2 | RPL35 |
| GNPTAB | RPL35A |
| GNS | RPL36 |
| GOLGA2 | RPL36A |
| GOLGA3 | RPL37 |
| GOLIM4 | RPL37A |
| GOLPH3 | RPL38 |
| GOLPH3L | RPL39 |
| GOPC | RPL5 |
| GORASP2 | RPL6 |
| GOSR1 | RPL7A |
| GPBP1 | RPL8 |
| GPBP1L1 | RPL9 |
| GPC6 | RPLP1 |
| GPD2 | RPLP2 |
| GPR107 | RPP21 |
| GPR125 | RPP30 |
| GPR126 | RPS10 |
| GPR155 | RPS11 |
| GPR89A | RPS13 |
| GRAMD3 | RPS14 |
| GRB2 | RPS15 |
| GRLF1 | RPS16 |
| GRSF1 | RPS17 |
| GSK3B | RPS19 |
| GSPT1 | RPS19BP1 |
| GTF2A1 | RPS2 |
| GTF2H3 | RPS20 |
| GTF2I | RPS21 |
| GTF3C1 | RPS23 |
| GTF3C2 | RPS25 |
| GTF3C3 | RPS26 |
| GTPBP1 | RPS27L |
| GUCY1A3 | RPS3 |
| GUF1 | RPS6 |
| H6PD | RPS6KB2 |
| HADHA | RPS7 |
| HAT1 | RPS8 |
| HBS1L | RPS9 |
| HCFC1 | RPSA |
| HCFC2 | RPUSD3 |
| HDAC2 | RRAS |
| HDHD2 | RRP7A |
| HDLBP | RRP7B |
| HEATR5B | RRP8 |
| HECTD1 | RRP9 |
| HECW2 | RTEL1 |
| HEG1 | RTN4IP1 |
| HELZ | RUVBL2 |
| HERC1 | RWDD3 |
| HERC3 | S100A10 |
| HERC4 | S100A11 |
| HERPUD2 | S100A13 |
| HFE | S100A6 |
| HGSNAT | SARNP |
| HIAT1 | SAT2 |
| HIATL1 | SCNM1 |
| HIF1A | SCO2 |
| HIF1AN | SCRN2 |
| HINT3 | SDF2L1 |
| HIP1 | SDR39U1 |
| HIPK1 | SEC13 |
| HIPK2 | SEC31B |
| HIPK3 | SEC61B |
| HIRA | SELK |
| HIVEP2 | SELM |
| HK1 | SELO |
| HK2 | 1-Sep |
| HLTF | SEPW1 |
| HMG20A | SEPX1 |
| HMGCR | SERF2 |
| HMGCS1 | SF3A2 |
| HMGXB3 | SFI1 |
| HMGXB4 | SFRS12IP1 |
| HN1L | SFRS16 |
| HNRNPF | SFRS8 |
| HNRNPK | SH3BGRL3 |
| HNRNPM | SHARPIN |
| HNRNPR | SHFM1 |
| HNRNPU | SIGIRR |
| HNRNPUL1 | SIRT6 |
| HOOK1 | SIRT7 |
| HP1BP3 | SIVA1 |
| HS2ST1 | SLC22A18 |
| HSD17B4 | SLC25A1 |
| HSP90AA1 | SLC25A39 |
| HSP90AB1 | SLC2A4RG |
| HSPA12A | SLC39A4 |
| HSPA13 | SMPD2 |
| HSPA4L | SNAPC2 |
| HSPC159 | SNHG6 |
| HTATSF1 | SNHG9 |
| HTT | SNORA8 |
| HUWE1 | SNRNP25 |
| HYOU1 | SNRNP35 |
| IARS | SNRPA |
| IARS2 | SNRPB |
| IBTK | SNRPB2 |
| ICMT | SNRPD2 |
| IDE | SNRPF |
| IDS | SNRPG |
| IFNAR1 | SOD1 |
| IFT88 | SPA17 |
| IGF1R | SPAG4 |
| IGF2R | SPSB3 |
| IKBKAP | SRA1 |
| IL13RA1 | SRM |
| IL17RA | SRP14 |
| IL1R1 | SS18L2 |
| IL4R | SSBP1 |
| IL6R | SSBP4 |
| IL6ST | SSNA1 |
| ILF3 | SSR4 |
| IMMT | SSSCA1 |
| IMPACT | SSU72 |
| INADL | STARD10 |
| ING3 | STOML1 |
| INO80 | STOML2 |
| INPP5A | STUB1 |
| INSR | STX10 |
| INTS6 | STX8 |
| IP6K1 | STYXL1 |
| IPO11 | SULT1A1 |
| IPO5 | SULT1A3 |
| IPO7 | SURF1 |
| IPO8 | SURF2 |
| IPO9 | SYCE1L |
| IQGAP1 | TACC3 |
| IQGAP2 | TAF10 |
| IQSEC1 | TALDO1 |
| IREB2 | TARBP1 |
| ITCH | TARBP2 |
| ITFG1 | TAX1BP3 |
| ITGA1 | TAZ |
| ITGA2 | TBC1D10C |
| ITGA4 | TBC1D24 |
| ITGA6 | TBC1D3B |
| ITGA8 | TBC1D3C |
| ITGAV | TBC1D3G |
| ITGB1 | TBC1D3H |
| ITGB8 | TBCA |
| ITSN1 | TBCB |
| ITSN2 | TBRG4 |
| JAG1 | TCEA2 |
| JAK1 | TCEB1 |
| JAZF1 | TCEB2 |
| JKAMP | TCTEX1D2 |
| JMJD1C | TECR |
| JOSD1 | TELO2 |
| JUB | TEX264 |
| KAT2B | TFPT |
| KAZ | THAP4 |
| KBTBD2 | THAP7 |
| KCNIP4 | THOC6 |
| KCNJ16 | THOC7 |
| KCNJ3 | THOP1 |
| KCNMA1 | THYN1 |
| KCTD10 | TIMM10 |
| KCTD18 | TIMM13 |
| KCTD2 | TIMM16 |
| KCTD20 | TIMM17B |
| KCTD3 | TIMM44 |
| KCTD5 | TIMM50 |
| KCTD9 | TM4SF5 |
| KDELC2 | TM7SF2 |
| KDM1B | TMED1 |
| KDM2A | TMED3 |
| KDM3A | TMEM115 |
| KDM3B | TMEM134 |
| KDM4A | TMEM141 |
| KDM4C | TMEM147 |
| KDM5A | TMEM149 |
| KDM5B | TMEM176A |
| KDM5C | TMEM179B |
| KDM6A | TMEM191A |
| KDR | TMEM205 |
| KDSR | TMEM208 |
| KHDRBS1 | TMEM219 |
| KHSRP | TMEM223 |
| KIAA0040 | TMEM39B |
| KIAA0090 | TMEM54 |
| KIAA0100 | TMEM60 |
| KIAA0146 | TMSB10 |
| KIAA0174 | TMUB1 |
| KIAA0196 | TNFRSF6B |
| KIAA0232 | TOMM40 |
| KIAA0247 | TOMM5 |
| KIAA0317 | TOMM6 |
| KIAA0319L | TP53I13 |
| KIAA0355 | TP53I3 |
| KIAA0368 | TP53TG1 |
| KIAA0427 | TPRA1 |
| KIAA0430 | TPRN |
| KIAA0494 | TRAPPC1 |
| KIAA0528 | TRAPPC2L |
| KIAA0562 | TRAPPC2P1 |
| KIAA0652 | TRAPPC4 |
| KIAA0776 | TRAPPC6A |
| KIAA0892 | TRMT1 |
| KIAA1012 | TRMT112 |
| KIAA1033 | TRMT2A |
| KIAA1109 | TRMU |
| KIAA1147 | TRPM4 |
| KIAA1191 | TRPT1 |
| KIAA1217 | TSEN54 |
| KIAA1267 | TSPO |
| KIAA1279 | TSSC4 |
| KIAA1370 | TST |
| KIAA1429 | TSTA3 |
| KIAA1430 | TSTD1 |
| KIAA1462 | TTLL1 |
| KIAA1598 | TUSC2 |
| KIAA1671 | TXN |
| KIAA1715 | TXNDC17 |
| KIDINS220 | TXNL4A |
| KIF13A | U2AF1L4 |
| KIF16B | UBA52 |
| KIF1B | UBE2M |
| KIF3B | UBE2S |
| KIF5B | UBL5 |
| KIFAP3 | UBL7 |
| KIRREL | UBXN1 |
| KITLG | UFD1L |
| KL | ULK3 |
| KLF11 | UQCR10 |
| KLF3 | UQCR11 |
| KLF6 | UQCRB |
| KLHDC10 | UQCRC1 |
| KLHL12 | UQCRQ |
| KLHL2 | UROD |
| KLHL20 | USE1 |
| KLHL24 | UXT |
| KLHL5 | VAMP5 |
| KLHL7 | VAMP8 |
| KLRAQ1 | VEGFB |
| KPNA1 | VPS28 |
| KPNA3 | WASH2P |
| KPNA4 | WASH3P |
| KPNA6 | WASH5P |
| KPNB1 | WASH7P |
| KRAS | WBSCR22 |
| KRR1 | WDR13 |
| KSR1 | WDR18 |
| L3MBTL2 | WDR24 |
| LACTB | WDR34 |
| LAMC1 | WDR45 |
| LAMP1 | WDR74 |
| LAMP2 | WDR83 |
| LANCL1 | WDR90 |
| LAPTM5 | WIBG |
| LARP1 | WRAP53 |
| LARP1B | XPNPEP3 |
| LARP4 | YDJC |
| LARP4B | YIF1A |
| LARS | YIF1B |
| LASP1 | YIPF2 |
| LASS2 | YPEL3 |
| LASS6 | ZBTB17 |
| LATS2 | ZBTB48 |
| LBR | ZBTB8OS |
| LCP1 | ZC3H3 |
| LEMD3 | ZDHHC12 |
| LEPR | ZDHHC24 |
| LEPROT | ZFAND2A |
| LGALS8 | ZFPL1 |
| LGR4 | ZMAT5 |
| LIFR | ZMYND19 |
| LIMCH1 | ZNF32 |
| LIMD1 | ZNF335 |
| LIN7C | ZNF414 |
| LIPA | ZNF428 |
| LIX1L | ZNF444 |
| LMAN1 | ZNF511 |
| LMAN2L | ZNF524 |
| LMBR1 | ZNF585A |
| LMBRD2 | ZNF593 |
| LNPEP | ZNF688 |
| LOC339524 | ZNF692 |
| LOC651250 | ZNF706 |
| LOC729082 | ZNF787 |
| LONP2 | ZNF8 |
| LPGAT1 | ZNHIT1 |
| LPHN2 | ZNRD1 |
| LPIN2 | ZP3 |
| LRBA | |
| LRCH1 | |
| LRCH3 | |
| LRIG1 | |
| LRIG3 | |
| LRP1 | |
| LRP10 | |
| LRP2 | |
| LRPPRC | |
| LRRC40 | |
| LRRC58 | |
| LRRK2 | |
| LSG1 | |
| LSM14A | |
| LTA4H | |
| LUZP6 | |
| LYN | |
| LYSMD3 | |
| M6PR | |
| MACF1 | |
| MADD | |
| MAGT1 | |
| MAK16 | |
| MALT1 | |
| MAML1 | |
| MAML2 | |
| MAN1A1 | |
| MAN1A2 | |
| MAN2A1 | |
| MAN2B2 | |
| MANBA | |
| MANEA | |
| MAP2K4 | |
| MAP3K1 | |
| MAP3K2 | |
| MAP3K3 | |
| MAP3K5 | |
| MAP3K7 | |
| MAP4 | |
| MAP4K4 | |
| MAP4K5 | |
| MAPK1 | |
| MAPK14 | |
| MAPK1IP1L | |
| MAPK6 | |
| MAPK8 | |
| MAPK9 | |
| MAPRE1 | |
| MAPRE2 | |
| 1-Mar | |
| 6-Mar | |
| 7-Mar | |
| 8-Mar | |
| MARK2 | |
| MARK4 | |
| MASP1 | |
| MAT2A | |
| MAT2B | |
| MATR3 | |
| MAVS | |
| MBD1 | |
| MBNL1 | |
| MBNL2 | |
| MBP | |
| MBTPS1 | |
| MBTPS2 | |
| MCCC1 | |
| MCCC2 | |
| MCFD2 | |
| MCM3 | |
| MCM3AP | |
| MDFIC | |
| MDM2 | |
| ME2 | |
| MED1 | |
| MED13 | |
| MED13L | |
| MED14 | |
| MED17 | |
| MED23 | |
| MED26 | |
| MEF2A | |
| MEF2C | |
| MEF2D | |
| MEGF8 | |
| MERTK | |
| MET | |
| METAP1 | |
| METAP2 | |
| METTL13 | |
| METTL14 | |
| MFAP1 | |
| MFAP3 | |
| MFN1 | |
| MFN2 | |
| MFSD1 | |
| MFSD11 | |
| MFSD6 | |
| MGAT4A | |
| MGAT5 | |
| MGEA5 | |
| MGLL | |
| MGRN1 | |
| MIA2 | |
| MIB1 | |
| MICAL2 | |
| MICAL3 | |
| MIDI | |
| MIER1 | |
| MINPP1 | |
| MITF | |
| MKLN1 | |
| MKRN1 | |
| MLL | |
| MLL3 | |
| MLL5 | |
| MLLT10 | |
| MLLT4 | |
| MLXIP | |
| MMGT1 | |
| MOBKL1B | |
| MOBKL2B | |
| MON1B | |
| MON2 | |
| MORC3 | |
| MPDZ | |
| MPP5 | |
| MSH2 | |
| MSI2 | |
| MSN | |
| MTAP | |
| MTDH | |
| MTFR1 | |
| MTIF2 | |
| MTMR1 | |
| MTMR10 | |
| MTMR12 | |
| MTMR2 | |
| MTMR3 | |
| MTMR6 | |
| MTO1 | |
| MTOR | |
| MTR | |
| MTRF1L | |
| MTRR | |
| MTSS1 | |
| MUT | |
| MUTED | |
| MXI1 | |
| MYCBP2 | |
| MYLIP | |
| MYLK | |
| MYO18A | |
| MYO1C | |
| MYOID | |
| MYO1E | |
| MYO5A | |
| MYO6 | |
| MYO9A | |
| MYOF | |
| MYST2 | |
| N4BP1 | |
| NAA15 | |
| NAA30 | |
| NAA35 | |
| NAA50 | |
| NAB1 | |
| NAF1 | |
| NARS | |
| NAT10 | |
| NBAS | |
| NBN | |
| NBPF1 | |
| NBR1 | |
| NCEH1 | |
| NCK2 | |
| NCKAP1 | |
| NCOA1 | |
| NCOA2 | |
| NCOA3 | |
| NCOA4 | |
| NCOA5 | |
| NCOA7 | |
| NCOR1 | |
| NCOR2 | |
| NCSTN | |
| NDFIP2 | |
| NDRG1 | |
| NDRG3 | |
| NDST1 | |
| NEBL | |
| NECAP1 | |
| NECAP2 | |
| NEDD1 | |
| NEDD4 | |
| NEDD9 | |
| NEK7 | |
| NEO1 | |
| NETO2 | |
| NF1 | |
| NFATC3 | |
| NFE2L1 | |
| NFE2L2 | |
| NFIA | |
| NFIB | |
| NFIX | |
| NFKB1 | |
| NFX1 | |
| NFYA | |
| NGLY1 | |
| NHLRC3 | |
| NID1 | |
| NIPA2 | |
| NIPAL2 | |
| NIPAL3 | |
| NIPBL | |
| NLK | |
| NLN | |
| NMD3 | |
| NMT2 | |
| NOL10 | |
| NOL11 | |
| NOLC1 | |
| NOMO1 | |
| NOMO2 | |
| NOMO3 | |
| NONO | |
| NOP14 | |
| NOTCH2 | |
| NPC1 | |
| NPEPPS | |
| NPLOC4 | |
| NR1D2 | |
| NR3C1 | |
| NRAS | |
| NRD1 | |
| NRP1 | |
| NSD1 | |
| NSF | |
| NSL1 | |
| NSMAF | |
| NSUN2 | |
| NT5C2 | |
| NUAK1 | |
| NUB1 | |
| NUDCD3 | |
| NUFIP2 | |
| NUMB | |
| NUP133 | |
| NUP153 | |
| NUP155 | |
| NUP160 | |
| NUP205 | |
| NUP214 | |
| NUP43 | |
| NUP50 | |
| NUP54 | |
| NUP98 | |
| NUPL1 | |
| NUS1 | |
| OAS3 | |
| OCRL | |
| OGDH | |
| OLFML2A | |
| OPA1 | |
| ORAI2 | |
| ORC2L | |
| OS9 | |
| OSBP | |
| OSBPL10 | |
| OSBPL1A | |
| OSBPL8 | |
| OSBPL9 | |
| OSGIN2 | |
| OSMR | |
| OTUD4 | |
| OTUD7B | |
| OXR1 | |
| OXSR1 | |
| P4HA1 | |
| PACSIN2 | |
| PAFAH1B1 | |
| PAFAH1B2 | |
| PAFAH2 | |
| PAG1 | |
| PAICS | |
| PAK1 | |
| PAK2 | |
| PAM | |
| PAN3 | |
| PANK1 | |
| PANK3 | |
| PAPD4 | |
| PAPOLA | |
| PAPSS1 | |
| PAPSS2 | |
| PARD3 | |
| PARG | |
| PARK2 | |
| PARN | |
| PARP1 | |
| PARP14 | |
| PARP4 | |
| PARP8 | |
| PATL1 | |
| PBLD | |
| PBX1 | |
| PCDHGB7 | |
| PCDHGC3 | |
| PCGF5 | |
| PCM1 | |
| PCMTD1 | |
| PCMTD2 | |
| PCNX | |
| PCYOX1 | |
| PCYT1A | |
| PDCD6IP | |
| PDCD7 | |
| PDCL | |
| PDE4A | |
| PDE4B | |
| PDE4D | |
| PDE7A | |
| PDE8A | |
| PDGFC | |
| PDK1 | |
| PDLIM5 | |
| PDPK1 | |
| PDS5A | |
| PDS5B | |
| PDSS2 | |
| PDXDC1 | |
| PDZD8 | |
| PELI1 | |
| PER3 | |
| PEX11B | |
| PEX26 | |
| PEX5 | |
| PGGT1B | |
| PGM2 | |
| PGM3 | |
| PGRMC2 | |
| PHACTR2 | |
| PHACTR4 | |
| PHAX | |
| PHF10 | |
| PHF15 | |
| PHF17 | |
| PHF2 | |
| PHF20 | |
| PHF20L1 | |
| PHF21A | |
| PHF3 | |
| PHF8 | |
| PHIP | |
| PHKB | |
| PHLDB2 | |
| PHRF1 | |
| PHTF1 | |
| PI4K2A | |
| PI4K2B | |
| PI4KA | |
| PIAS1 | |
| PICALM | |
| PIGG | |
| PIGK | |
| PIGN | |
| PIGS | |
| PIGX | |
| PIK3AP1 | |
| PIK3C2A | |
| PIK3C3 | |
| PIK3CA | |
| PIK3CB | |
| PIK3R1 | |
| PIK3R3 | |
| PIKFYVE | |
| PIP4K2A | |
| PIP4K2B | |
| PIP4K2C | |
| PIP5K1A | |
| PIP5K1C | |
| PITPNA | |
| PITPNB | |
| PITRM1 | |
| PJA2 | |
| PKD2 | |
| PKN2 | |
| PKP2 | |
| PKP4 | |
| PLAA | |
| PLBD2 | |
| PLCB1 | |
| PLD1 | |
| PLDN | |
| PLEC | |
| PLEKHA1 | |
| PLEKHA2 | |
| PLEKHA3 | |
| PLEKHA7 | |
| PLEKHG1 | |
| PLEKHO2 | |
| PLS1 | |
| PLSCR4 | |
| PLXDC2 | |
| PLXNA2 | |
| PLXNC1 | |
| PM20D2 | |
| PMS2 | |
| PNPLA3 | |
| PNRC2 | |
| POC1B | |
| PODXL | |
| POFUT1 | |
| POGK | |
| POLD3 | |
| POLDIP3 | |
| POLH | |
| POLK | |
| POLR2A | |
| POLR2B | |
| POM121 | |
| POM121C | |
| PPAP2B | |
| PPARD | |
| PPFIA1 | |
| PPFIBP1 | |
| PPIG | |
| PPIP5K2 | |
| PPM1B | |
| PPM1D | |
| PPM1F | |
| PPME1 | |
| PPP1CB | |
| PPP1CC | |
| PPP1R10 | |
| PPP1R12A | |
| PPP1R16B | |
| PPP1R8 | |
| PPP2CB | |
| PPP2R1B | |
| PPP2R2A | |
| PPP2R3A | |
| PPP2R5D | |
| PPP2R5E | |
| PPP3CA | |
| PPP3R1 | |
| PPP4R1 | |
| PPP4R2 | |
| PPP6C | |
| PPT1 | |
| PPTC7 | |
| PRCP | |
| PRDM4 | |
| PREPL | |
| PREX1 | |
| PRKAA1 | |
| PRKAA2 | |
| PRKACB | |
| PRKAR1A | |
| PRKAR2A | |
| PRKCI | |
| PRKD1 | |
| PRKD3 | |
| PRKDC | |
| PRKX | |
| PRMT5 | |
| PROSC | |
| PRPF18 | |
| PRPF4 | |
| PRPF40A | |
| PRPF4B | |
| PRPF8 | |
| PRRC1 | |
| PRUNE | |
| PRUNE2 | |
| PSAP | |
| PSEN1 | |
| PSIP1 | |
| PSMD1 | |
| PSMD12 | |
| PSMD5 | |
| PSME4 | |
| PTAR1 | |
| PTBP1 | |
| PTDSS1 | |
| PTEN | |
| PTK2 | |
| PTP4A1 | |
| PTPLB | |
| PTPN1 | |
| PTPN11 | |
| PTPN12 | |
| PTPN13 | |
| PTPN14 | |
| PTPN23 | |
| PTPN9 | |
| PTPRB | |
| PTPRE | |
| PTPRG | |
| PTPRJ | |
| PTPRK | |
| PTPRM | |
| PUM1 | |
| PUM2 | |
| PVRL3 | |
| PWP1 | |
| PXN | |
| PYROXD1 | |
| QKI | |
| QRICH1 | |
| R3HDM1 | |
| R3HDM2 | |
| RAB10 | |
| RAB12 | |
| RAB14 | |
| RAB18 | |
| RAB1A | |
| RAB21 | |
| RAB31 | |
| RAB35 | |
| RAB36 | |
| RAB3GAP1 | |
| RAB3GAP2 | |
| RAB5A | |
| RAB5B | |
| RAB6A | |
| RAB7L1 | |
| RAB8B | |
| RABEP1 | |
| RABGAP1 | |
| RABGEF1 | |
| RABL3 | |
| RAD17 | |
| RAD21 | |
| RAD23B | |
| RAD50 | |
| RAI14 | |
| RALB | |
| RALBP1 | |
| RALGAPA2 | |
| RALGAPB | |
| RANBP10 | |
| RANBP2 | |
| RANBP9 | |
| RAP1A | |
| RAP1B | |
| RAPGEF1 | |
| RAPGEF2 | |
| RAPGEF5 | |
| RARS | |
| RASA1 | |
| RASA3 | |
| RASGRP1 | |
| RASSF2 | |
| RASSF3 | |
| RAVER2 | |
| RB1 | |
| RB1CC1 | |
| RBBP4 | |
| RBBP5 | |
| RBBP9 | |
| RBL2 | |
| RBM12 | |
| RBM16 | |
| RBM18 | |
| RBM22 | |
| RBM23 | |
| RBM26 | |
| RBM27 | |
| RBM47 | |
| RBM7 | |
| RBMS1 | |
| RBMS2 | |
| RBPJ | |
| RC3H2 | |
| RCBTB1 | |
| RCBTB2 | |
| RCC2 | |
| RCHY1 | |
| RCOR1 | |
| RCOR3 | |
| RCSD1 | |
| RDX | |
| RECQL | |
| REEP3 | |
| RELL1 | |
| REPS1 | |
| RERE | |
| RETSAT | |
| RFC1 | |
| RFFL | |
| RFX5 | |
| RGL1 | |
| RGNEF | |
| RGP1 | |
| RHBDD1 | |
| RHOA | |
| RHOBTB1 | |
| RHOBTB3 | |
| RHOT1 | |
| RHOU | |
| RHPN2 | |
| RIF1 | |
| RIN2 | |
| RIOK2 | |
| RIOK3 | |
| RIPK1 | |
| RIT1 | |
| RMND5A | |
| RNASEN | |
| RNF103 | |
| RNF11 | |
| RNF111 | |
| RNF115 | |
| RNF121 | |
| RNF128 | |
| RNF13 | |
| RNF138 | |
| RNF141 | |
| RNF144B | |
| RNF145 | |
| RNF146 | |
| RNF160 | |
| RNF170 | |
| RNF185 | |
| RNF19B | |
| RNF20 | |
| RNF216 | |
| RNF38 | |
| RNF4 | |
| RNF40 | |
| RNF6 | |
| RNMT | |
| ROCK1 | |
| ROCK2 | |
| ROD1 | |
| RORA | |
| RP2 | |
| RPA1 | |
| RPE | |
| RPL7L1 | |
| RPRD1A | |
| RPRD1B | |
| RPRD2 | |
| RPS6KA2 | |
| RPS6KA3 | |
| RPS6KB1 | |
| RPUSD4 | |
| RRM1 | |
| RRM2B | |
| RRN3 | |
| RRP1B | |
| RSBN1L | |
| RSF1 | |
| RSPRY1 | |
| RTN3 | |
| RUNDC2A | |
| RYBP | |
| RYK | |
| SAMD4B | |
| SAMHD1 | |
| SAP130 | |
| SAPS3 | |
| SART3 | |
| SASH1 | |
| SAV1 | |
| SBF2 | |
| SBNO1 | |
| SCAMP1 | |
| SCAPER | |
| SCARB2 | |
| SCD | |
| SCP2 | |
| SCRN1 | |
| SCRN3 | |
| SCYL2 | |
| SDAD1 | |
| SDCBP | |
| SDCCAG8 | |
| SEC14L1 | |
| SEC16A | |
| SEC22B | |
| SEC23A | |
| SEC23B | |
| SEC23IP | |
| SEC24A | |
| SEC24B | |
| SEC24C | |
| SEC24D | |
| SEC31A | |
| SEC61A1 | |
| SEC63 | |
| SECISBP2L | |
| SEH1L | |
| SEL1L | |
| SEMA4D | |
| SEMA5A | |
| SEMA6A | |
| SENP2 | |
| SENP6 | |
| SEPN1 | |
| SEPP1 | |
| SEPSECS | |
| 10-Sep | |
| 11-Sep | |
| 2-Sep | |
| 7-Sep | |
| 8-Sep | |
| 9-Sep | |
| SERINC1 | |
| SERINC3 | |
| SERINC5 | |
| SERPINB9 | |
| SESTD1 | |
| SETD3 | |
| SETD7 | |
| SETX | |
| SF1 | |
| SF3A1 | |
| SF3A3 | |
| SF3B3 | |
| SFRS13A | |
| SFRS2IP | |
| SFXN1 | |
| SFXN3 | |
| SGK3 | |
| SGMS1 | |
| SGMS2 | |
| SGPL1 | |
| SGPP2 | |
| SGSH | |
| SH2B3 | |
| SH3BGRL2 | |
| SH3D19 | |
| SHOC2 | |
| SHROOM4 | |
| SIAE | |
| SIK2 | |
| SIK3 | |
| SIN3A | |
| SIPA1L2 | |
| SIRT1 | |
| SKAP2 | |
| SKI | |
| SKIL | |
| SKIV2L2 | |
| SLAIN2 | |
| SLC11A2 | |
| SLC12A2 | |
| SLC12A6 | |
| SLC12A7 | |
| SLC12A8 | |
| SLC15A4 | |
| SLC16A1 | |
| SLC16A4 | |
| SLC17A5 | |
| SLC1A1 | |
| SLC1A3 | |
| SLC20A2 | |
| SLC23A2 | |
| SLC25A13 | |
| SLC25A24 | |
| SLC25A30 | |
| SLC25A36 | |
| SLC25A44 | |
| SLC25A46 | |
| SLC30A5 | |
| SLC30A6 | |
| SLC30A7 | |
| SLC30A9 | |
| SLC33A1 | |
| SLC35A3 | |
| SLC35A5 | |
| SLC35B3 | |
| SLC35B4 | |
| SLC35D1 | |
| SLC35E1 | |
| SLC35F5 | |
| SLC37A2 | |
| SLC37A3 | |
| SLC38A1 | |
| SLC38A2 | |
| SLC39A10 | |
| SLC39A8 | |
| SLC39A9 | |
| SLC40A1 | |
| SLC41A1 | |
| SLC41A2 | |
| SLC4A4 | |
| SLC6A6 | |
| SLC8A1 | |
| SLCO2B1 | |
| SLCO4C1 | |
| SLFN5 | |
| SLK | |
| SLMAP | |
| SLU7 | |
| SMAD3 | |
| SMAD4 | |
| SMAD5 | |
| SMAP1 | |
| SMAP2 | |
| SMARCA1 | |
| SMARCA2 | |
| SMARCA5 | |
| SMARCAD1 | |
| SMARCC1 | |
| SMARCC2 | |
| SMARCD1 | |
| SMC1A | |
| SMC3 | |
| SMCHD1 | |
| SMCR7L | |
| SMEK1 | |
| SMEK2 | |
| SMG1 | |
| SMG6 | |
| SMG7 | |
| SMPD4 | |
| SMU1 | |
| SMURF1 | |
| SMURF2 | |
| SNAP23 | |
| SNAPC3 | |
| SNRK | |
| SNRNP200 | |
| SNX1 | |
| SNX10 | |
| SNX12 | |
| SNX13 | |
| SNX19 | |
| SNX2 | |
| SNX25 | |
| SNX27 | |
| SNX29 | |
| SNX30 | |
| SNX4 | |
| SNX6 | |
| SNX9 | |
| SOAT1 | |
| SON | |
| SORBS1 | |
| SORBS2 | |
| SORL1 | |
| SORT1 | |
| SOS1 | |
| SOS2 | |
| SP1 | |
| SP2 | |
| SP3 | |
| SPAG9 | |
| SPAST | |
| SPATA13 | |
| SPATA18 | |
| SPATA6 | |
| SPATS2 | |
| SPDYA | |
| SPG11 | |
| SPG20 | |
| SPIN1 | |
| SPIRE1 | |
| SPOP | |
| SPOPL | |
| SPPL2A | |
| SPPL3 | |
| SPRED1 | |
| SPRED2 | |
| SPTAN1 | |
| SPTBN1 | |
| SPTLC1 | |
| SPTY2D1 | |
| SR140 | |
| SRBD1 | |
| SRCAP | |
| SRFBP1 | |
| SRGAP2 | |
| SRP68 | |
| SRP72 | |
| SRP9 | |
| SRPK1 | |
| SRPK2 | |
| SRPR | |
| SRRM1 | |
| SS18 | |
| SSFA2 | |
| SSR1 | |
| SSX2IP | |
| ST13 | |
| ST3GAL1 | |
| ST8SIA4 | |
| STAG1 | |
| STAG2 | |
| STAM | |
| STAM2 | |
| STARD13 | |
| STARD7 | |
| STARD8 | |
| STAT3 | |
| STAT5B | |
| STAT6 | |
| STAU1 | |
| STAU2 | |
| STK10 | |
| STK17B | |
| STK24 | |
| STK32B | |
| STK38 | |
| STK38L | |
| STK4 | |
| STK40 | |
| STOM | |
| STRN | |
| STRN3 | |
| STS | |
| STT3A | |
| STT3B | |
| STX12 | |
| STX17 | |
| STX2 | |
| STX3 | |
| STX6 | |
| STX7 | |
| STXBP3 | |
| SUCLG2 | |
| SUDS3 | |
| SUFU | |
| SUN1 | |
| SUN2 | |
| SUPT16H | |
| SUPT6H | |
| SUV420H1 | |
| SUZ12 | |
| SWAP70 | |
| SYK | |
| SYNCRIP | |
| SYNE1 | |
| SYNE2 | |
| SYNM | |
| SYNRG | |
| SYPL1 | |
| TAB1 | |
| TAB2 | |
| TAB3 | |
| TACC1 | |
| TAF1 | |
| TAF1B | |
| TAF2 | |
| TAF9B | |
| TANC1 | |
| TANK | |
| TAOK1 | |
| TAOK3 | |
| TAPBP | |
| TAPT1 | |
| TARDBP | |
| TARSL2 | |
| TBC1D13 | |
| TBC1D15 | |
| TBC1D16 | |
| TBC1D19 | |
| TBC1D20 | |
| TBC1D22B | |
| TBC1D23 | |
| TBC1D2B | |
| TBC1D5 | |
| TBC1D8B | |
| TBC1D9 | |
| TBC1D9B | |
| TBCK | |
| TBK1 | |
| TBL1X | |
| TBL1XR1 | |
| TCEA1 | |
| TCF12 | |
| TCF7L2 | |
| TCHP | |
| TCTN2 | |
| TCTN3 | |
| TDG | |
| TDP2 | |
| TEAD1 | |
| TERF1 | |
| TERF2 | |
| TEX2 | |
| TEX261 | |
| TFCP2 | |
| TFDP1 | |
| TFRC | |
| TGFA | |
| TGFBR1 | |
| TGFBR2 | |
| TGFBR3 | |
| TGFBRAP1 | |
| TGOLN2 | |
| TGS1 | |
| THBS1 | |
| THOC2 | |
| THRAP3 | |
| THUMPD1 | |
| TIMM17A | |
| TJP1 | |
| TJP2 | |
| TK2 | |
| TLE3 | |
| TLK1 | |
| TLK2 | |
| TLN1 | |
| TLN2 | |
| TLR3 | |
| TLR4 | |
| TM7SF3 | |
| TM9SF2 | |
| TM9SF3 | |
| TM9SF4 | |
| TMCC3 | |
| TMCO3 | |
| TMED2 | |
| TMED5 | |
| TMED7-TICAM2 | |
| TMED7 | |
| TMEM123 | |
| TMEM127 | |
| TMEM131 | |
| TMEM135 | |
| TMEM144 | |
| TMEM150C | |
| TMEM167B | |
| TMEM181 | |
| TMEM184B | |
| TMEM185A | |
| TMEM192 | |
| TMEM2 | |
| TMEM200A | |
| TMEM209 | |
| TMEM30A | |
| TMEM41B | |
| TMEM43 | |
| TMEM48 | |
| TMEM57 | |
| TMEM63B | |
| TMEM66 | |
| TMEM87A | |
| TMEM87B | |
| TMF1 | |
| TMOD3 | |
| TMPO | |
| TMTC1 | |
| TMTC2 | |
| TMTC3 | |
| TMX1 | |
| TMX3 | |
| TNFAIP1 | |
| TNFRSF10A | |
| TNFRSF19 | |
| TNFRSF1B | |
| TNKS | |
| TNKS2 | |
| TNNI3K | |
| TNPO1 | |
| TNPO2 | |
| TNPO3 | |
| TNS1 | |
| TNS3 | |
| TOM1L2 | |
| TOP1 | |
| TOP2B | |
| TOR1AIP1 | |
| TOR1B | |
| TOX4 | |
| TP53 | |
| TP53BP2 | |
| TPMT | |
| TPP1 | |
| TPP2 | |
| TPR | |
| TPRG1L | |
| TRA2B | |
| TRAF3IP1 | |
| TRAF6 | |
| TRAK2 | |
| TRAM1 | |
| TRAM2 | |
| TRAPPC10 | |
| TRAPPC6B | |
| TRIM23 | |
| TRIM25 | |
| TRIM26 | |
| TRIM33 | |
| TRIM4 | |
| TRIM44 | |
| TRIM56 | |
| TRIO | |
| TRIP12 | |
| TRPM7 | |
| TRRAP | |
| TSN | |
| TSNAX-DISC1 | |
| TSNAX | |
| TSPAN12 | |
| TSPAN9 | |
| TSR1 | |
| TTC17 | |
| TTC19 | |
| TTC28 | |
| TTC3 | |
| TTC33 | |
| TTC37 | |
| TUBB | |
| TUBGCP3 | |
| TUG1 | |
| TULP3 | |
| TXLNA | |
| TXLNG | |
| TXNDC5 | |
| TXNIP | |
| TYW1 | |
| UBA6 | |
| UBE2D3 | |
| UBE2G1 | |
| UBE2G2 | |
| UBE2H | |
| UBE2J1 | |
| UBE2W | |
| UBE2Z | |
| UBE3A | |
| UBE3B | |
| UBE3C | |
| UBE4A | |
| UBE4B | |
| UBLCP1 | |
| UBN1 | |
| UBP1 | |
| UBQLN1 | |
| UBR1 | |
| UBR2 | |
| UBR3 | |
| UBR4 | |
| UBR5 | |
| UBR7 | |
| UBTD2 | |
| UBXN2B | |
| UBXN4 | |
| UCHL5 | |
| UEVLD | |
| UGCG | |
| UGGT1 | |
| UGT2A3 | |
| UHMK1 | |
| ULK2 | |
| UNC119B | |
| UNC13B | |
| UNC5B | |
| UNG | |
| UPF1 | |
| UPF2 | |
| UPRT | |
| USO1 | |
| USP1 | |
| USP10 | |
| USP12 | |
| USP14 | |
| USP15 | |
| USP16 | |
| USP19 | |
| USP22 | |
| USP24 | |
| USP25 | |
| USP3 | |
| USP30 | |
| USP32 | |
| USP33 | |
| USP34 | |
| USP36 | |
| USP38 | |
| USP40 | |
| USP47 | |
| USP48 | |
| USP53 | |
| USP7 | |
| USP8 | |
| USP9X | |
| UTRN | |
| UVRAG | |
| VAMP3 | |
| VAMP7 | |
| VAPB | |
| VAV2 | |
| VAV3 | |
| VCL | |
| VEZF1 | |
| VEZT | |
| VIPAR | |
| VPS13C | |
| VPS24 | |
| VPS26A | |
| VPS35 | |
| VPS36 | |
| VPS39 | |
| VPS41 | |
| VPS4B | |
| VPS52 | |
| VPS54 | |
| VRK2 | |
| VTA1 | |
| VWA5A | |
| WAC | |
| WAPAL | |
| WARS2 | |
| WASF2 | |
| WASL | |
| WBP11 | |
| WDFY1 | |
| WDFY3 | |
| WDR11 | |
| WDR26 | |
| WDR3 | |
| WDR33 | |
| WDR36 | |
| WDR43 | |
| WDR45L | |
| WDR48 | |
| WDR55 | |
| WDR72 | |
| WDR82 | |
| WDTC1 | |
| WHSC1L1 | |
| WIPF1 | |
| WIPF2 | |
| WNK1 | |
| WRB | |
| WWC2 | |
| WWC3 | |
| WWOX | |
| WWP1 | |
| WWP2 | |
| WWTR1 | |
| XIAP | |
| XPC | |
| XPNPEP1 | |
| XPO1 | |
| XPO5 | |
| XPO6 | |
| XPO7 | |
| XPR1 | |
| XRCC5 | |
| XRCC6 | |
| XRN1 | |
| XRN2 | |
| YAP1 | |
| YEATS2 | |
| YES1 | |
| YIPF5 | |
| YIPF6 | |
| YLPM1 | |
| YME1L1 | |
| YPEL2 | |
| YTHDC1 | |
| YTHDC2 | |
| YTHDF1 | |
| YTHDF2 | |
| YTHDF3 | |
| YWHAZ | |
| YY1 | |
| YY1AP1 | |
| YY2 | |
| ZAK | |
| ZBTB10 | |
| ZBTB38 | |
| ZBTB4 | |
| ZBTB44 | |
| ZC3H11A | |
| ZC3H13 | |
| ZC3H15 | |
| ZC3H4 | |
| ZC3H7A | |
| ZC3H7B | |
| ZC3HAV1 | |
| ZCCHC14 | |
| ZCCHC8 | |
| ZDHHC17 | |
| ZDHHC20 | |
| ZDHHC5 | |
| ZDHHC7 | |
| ZDHHC9 | |
| ZEB1 | |
| ZEB2 | |
| ZFAND1 | |
| ZFAND3 | |
| ZFP106 | |
| ZFP64 | |
| ZFP91-CNTF | |
| ZFP91 | |
| ZFR | |
| ZFYVE16 | |
| ZFYVE9 | |
| ZKSCAN1 | |
| ZMIZ1 | |
| ZMPSTE24 | |
| ZMYM2 | |
| ZMYM3 | |
| ZMYM4 | |
| ZMYND11 | |
| ZNF12 | |
| ZNF146 | |
| ZNF148 | |
| ZNF185 | |
| ZNF217 | |
| ZNF24 | |
| ZNF25 | |
| ZNF280D | |
| ZNF282 | |
| ZNF302 | |
| ZNF331 | |
| ZNF33A | |
| ZNF33B | |
| ZNF362 | |
| ZNF395 | |
| ZNF496 | |
| ZNF512 | |
| ZNF592 | |
| ZNF638 | |
| ZNF639 | |
| ZNF664 | |
| ZNF704 | |
| ZNF740 | |
| ZNF770 | |
| ZNFX1 | |
| ZRANB1 | |
| ZRANB2 | |
| ZW10 | |
| ZXDC | |
| ZYG11B | |
| ZZEF1 | |
| ZZZ3 | |

Strikingly, in tumor samples that displayed TS, some genes were characterized by interspersed expression of short intronic regions without any apparent exon expression (see FIG. 3E and FIG. 4), indicating spurious cryptic transcription. By mapping RNAseq reads onto exon-exon and the corresponding exon-intron junctions, genome-wide intron retention and poor exon definition were quantified, finding that tumors with TS had widespread defects in intron splicing, especially in the Type I genes (FIG. 1E-G). There was no significant difference in the number of reads mapping to intergenic regions, indicating that intron retention in tumors with TS is not an artifact of DNA contamination.

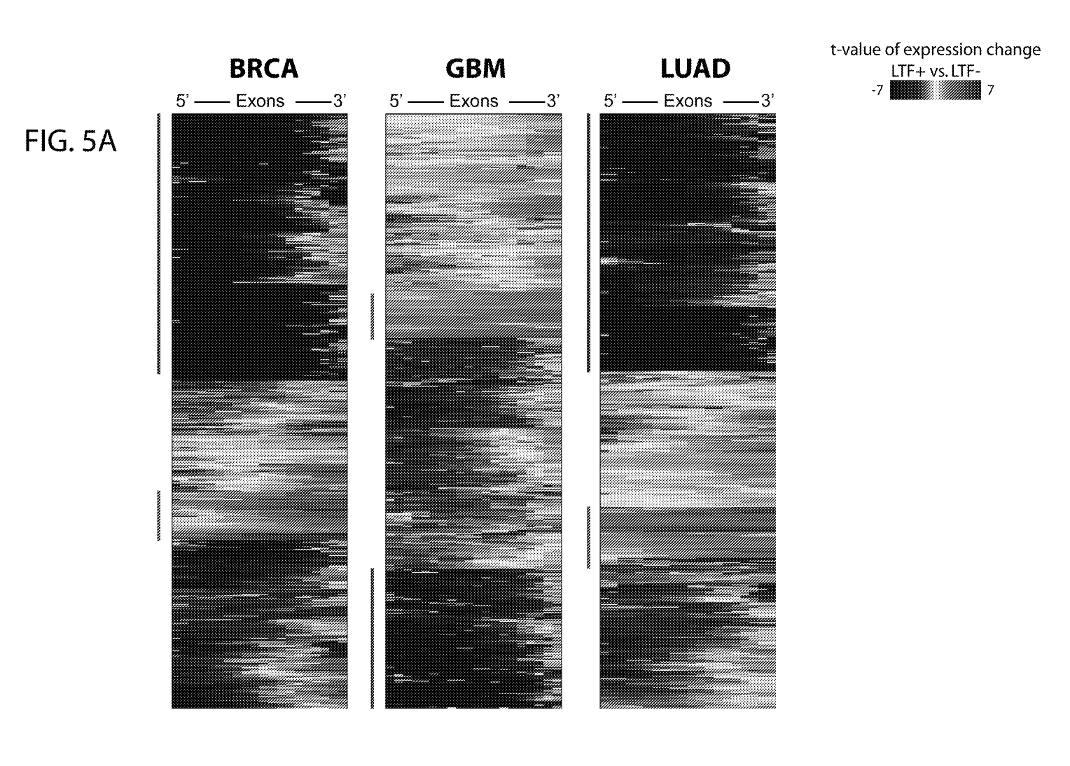

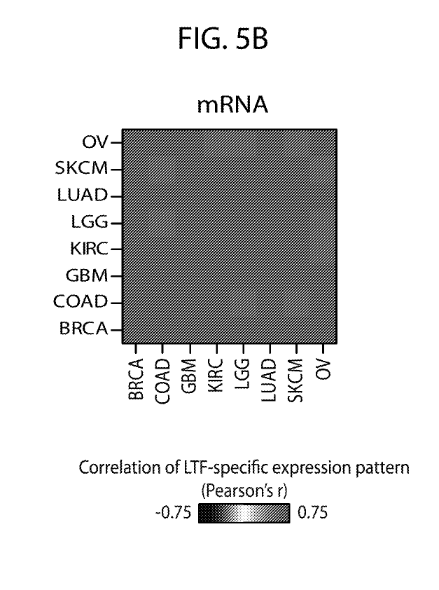

Widespread intron retention and spurious transcription indicate that TS is a phenotype of widespread loss of transcriptional fidelity (LTF), and, importantly, that the 5′ shortening in mRNAs is not an artifact of RNA degradation, but of severely defective RNA polymerase II transcriptional machinery. Remarkably, the transcript and exon-level expression patterns were highly consistent among LTF+ tumors of different cancers (FIG. 5), indicating that the LTF phenotype is highly conserved across tissues and imposes a well-defined aberrant molecular profile.

### Example 3

### LTF is Observed in Cancer Cell Lines and Involves Defective mRNA Transcription Initiation, Elongation and Processing

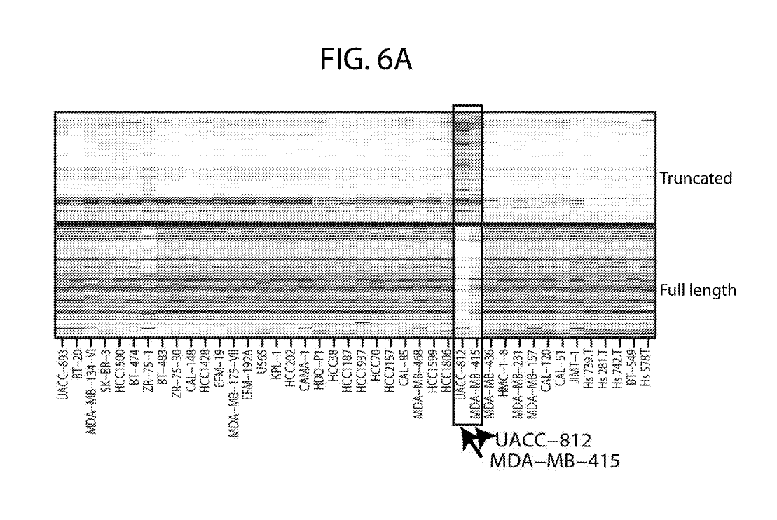

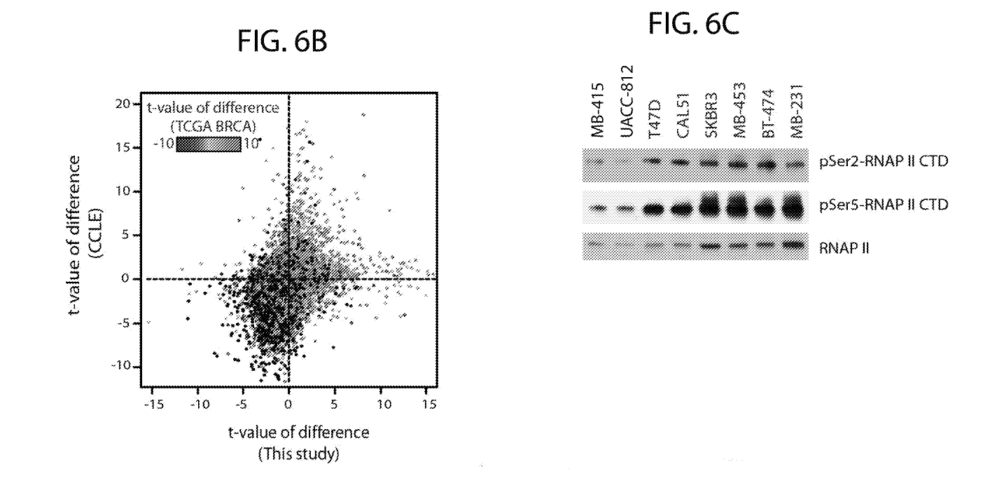

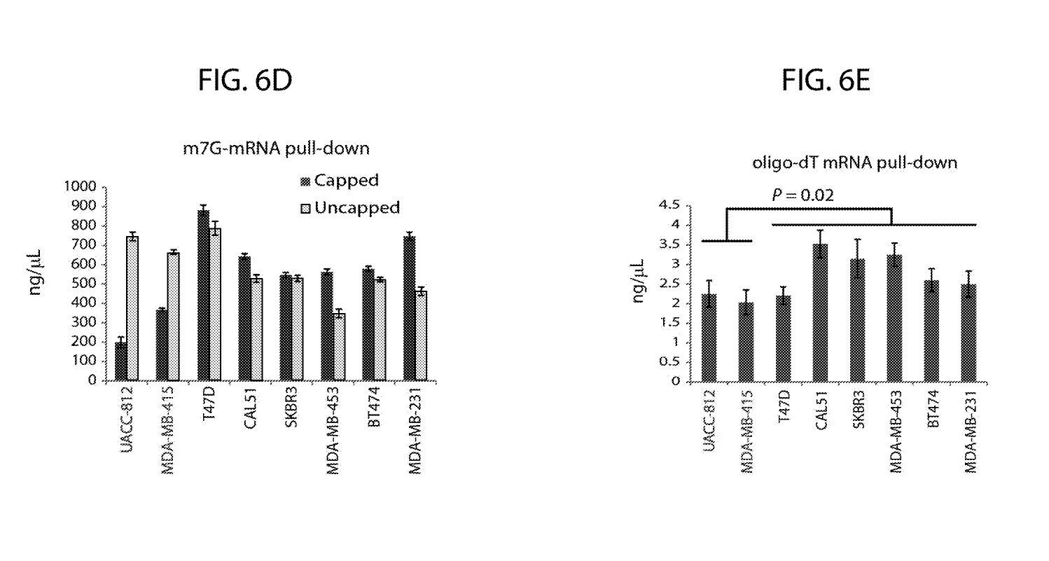

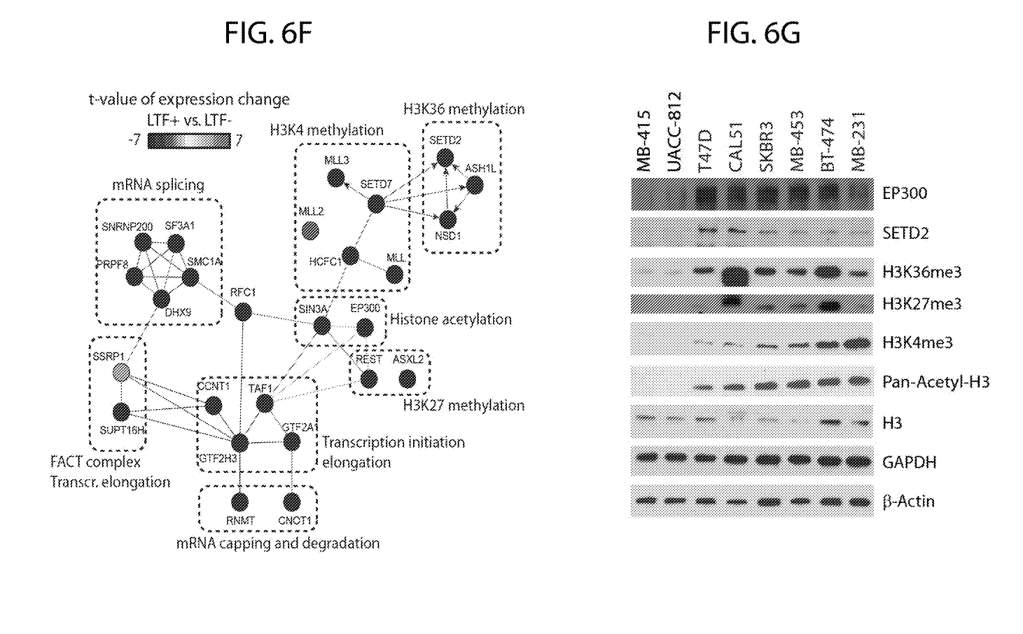

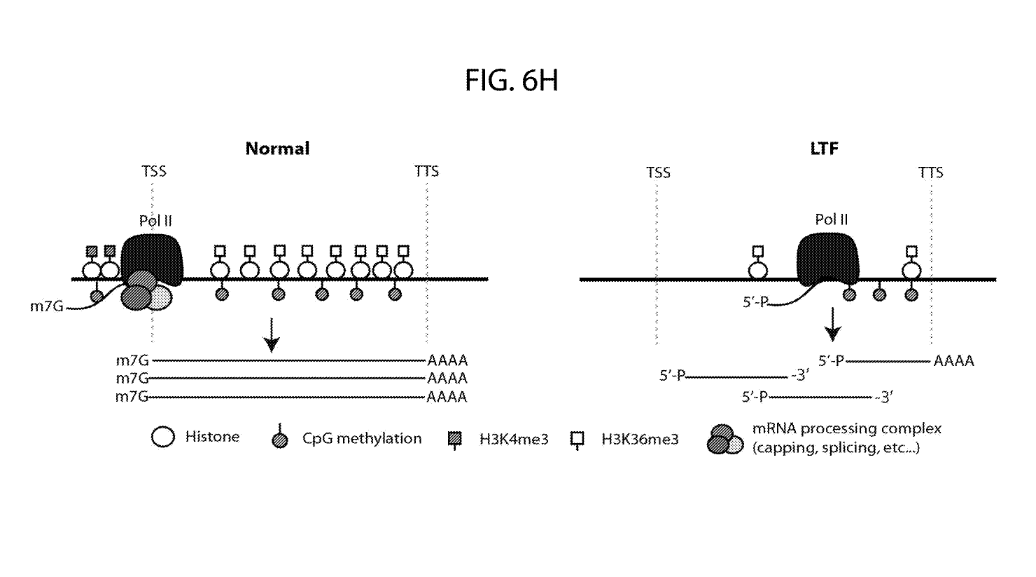

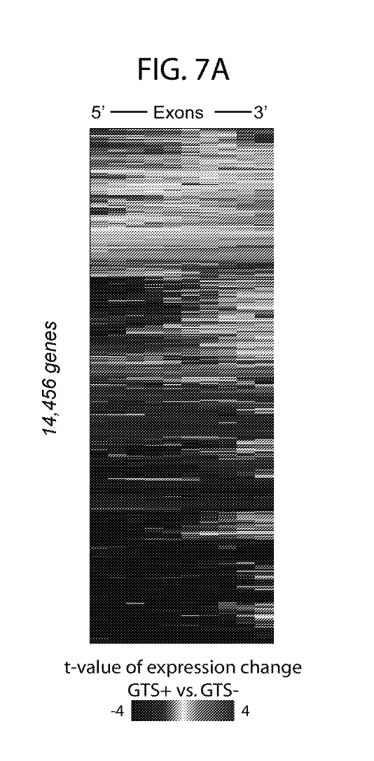

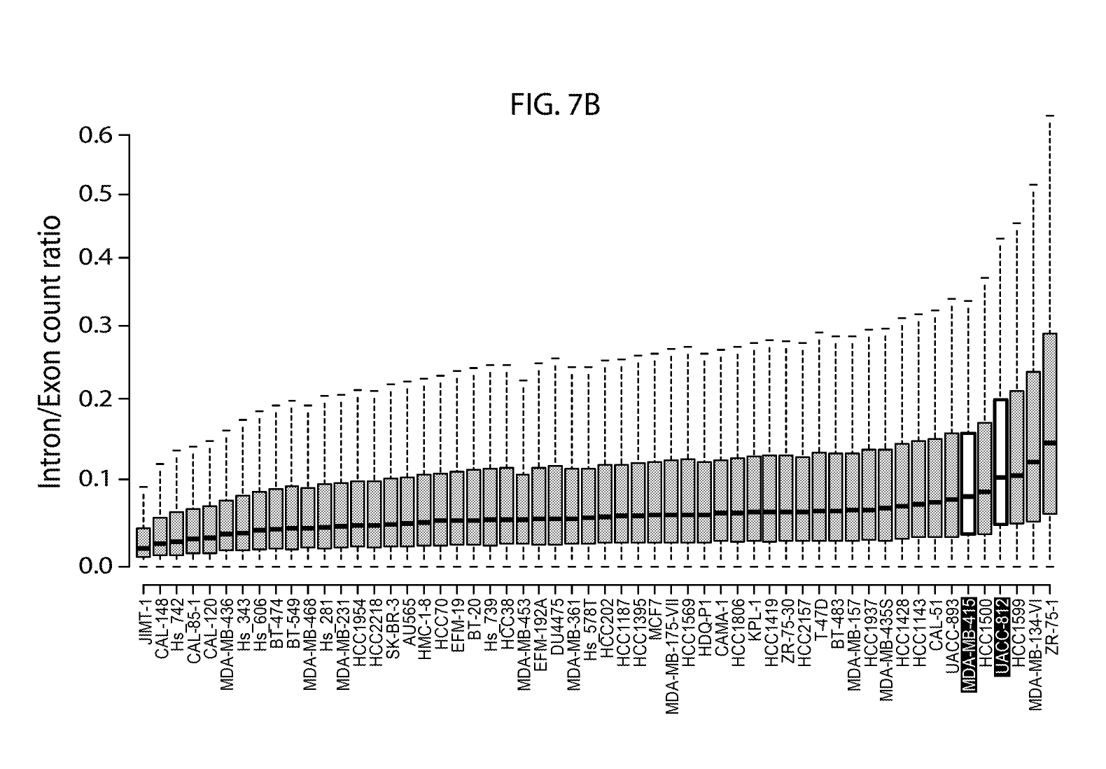

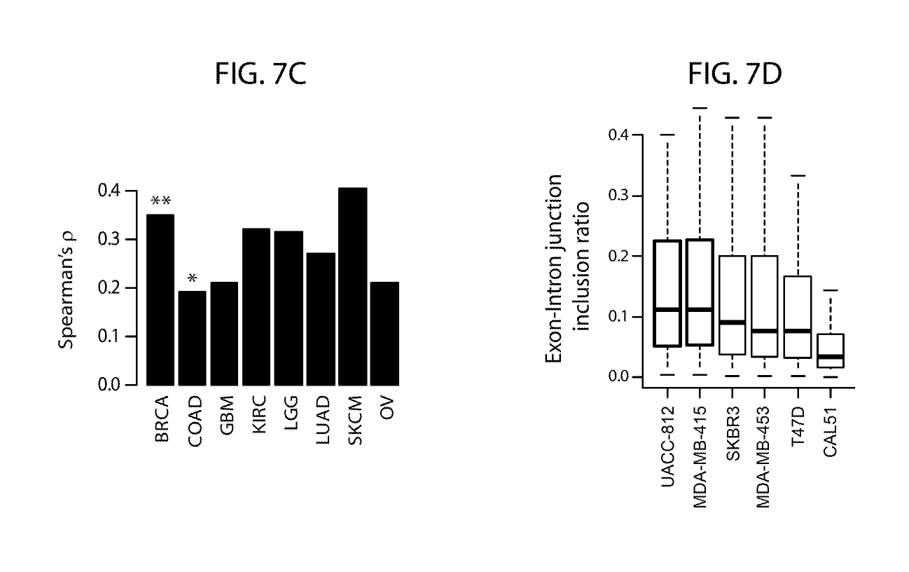

Through a similar analysis of RNA sequencing data from a panel of breast cancer cell lines, it was found that two lines (UACC-812 and MDA-MB-415), displayed a transcript shortening phenotype consistent with LTF in clinical datasets from TCGA (FIG. 6A). The differential exon expression heatmap revealed widespread 5′ shortening in these two lines, again consistent with the TCGA samples (FIG. 7A), and increased global intron retention (FIG. 7B). The overall gene expression profile of these cell lines relative to LTF− cells (i.e. t-values of difference) was also highly similar to that of LTF+ clinical samples (FIG. 7C).

In order to rule out a possibility that a technical artifact of RNA sequencing in TCGA and Cancer Cell Line Encyclopedia (CCLE) samples could have caused the LTF-like phenotype, independent RNA-seq analyses of these and several other breast cancer cell lines were performed. Importantly, the differential gene expression profile of UACC-812 and MDA-MB-415 cells relative to other cells in this experiment was highly similar to the similar analysis in CCLE samples, and more importantly, to the LTF− specific profiles observed in TCGA samples (FIG. 6B and FIG. 7D). These observations strongly indicate that UACC-812 and MDA-MB-415 cells display a LTF-like phenotype that is highly consistent with the LTF phenotype seen in patient samples.

The cryptic expression profile in LTF+ cells indicates severe defects in RNAP II transcription initiation and elongation functions. During transcription initiation, RNAP II is phosphorylated at the Ser5 position of its C-terminal domain (CTD), and later at the Ser2 position in the elongation phase, which is mediated by CCNT1/CDK9 (p-TEFb complex) (Jonkers and Lis, 2015). Interestingly, UACC-812 and MDA-MB-415 cells had significantly reduced levels of RNAP II CTD phosphorylation at both Ser5 and Ser2 positions (FIG. 6E), indicating that transcription initiation and elongation functions of RNAP II are defective in these cells. An important function of RNAP II CTD phosphorylation is to recruit various transcription-associated complexes required for mRNA capping and splicing, histone remodeling, and transcript elongation (Ho and Shuman, 1999; Jonkers and Lis, 2015; Nilson et al., 2015; Venkatesh and Workman, 2015).

Consistent with defective transcription and mRNA splicing in LTF+ tumor cells, the present biochemical analyses showed that UACC-812 and MDA-MB-415 cells were also defective in mRNA 5′-capping and 3′-poly-adenylation (FIG. 6D-E). Therefore, UACC-812 and MDA-MB-415 cells display a LTF phenotype highly consistent with LTF+ cancer tissues, characterized by defective mRNA transcription and processing machineries.

### Example 4

### LTF is Associated with Defective Chromatin Remodeling

Widespread intragenic cryptic transcription has been reported in yeasts and human cells with impaired gene body chromatin remodeling and transcription elongation machineries (Carrozza et al., 2005; Carvalho et al., 2013; Cheung et al., 2008; Kaplan et al., 2003; Venkatesh and Workman, 2015; Xie et al., 2011). Indeed, the present network-based analyses of the most consistent gene expression changes in LTF+ tumors across different lineages revealed that genes involved in chromatin remodeling, histone H3 methylation at K4, K27 and K36, as well as histone acetylations, demethylations and RNAP II transcription initiation and elongation, were consistently the most downregulated genes in LTF+ tumors (FIG. 6F), all of which have important roles in promoting proper transcription and splicing, and suppressing spurious transcription (Carrozza et al., 2005; Carvalho et al., 2013; Cheung et al., 2008; Kaplan et al., 2003; Mason and Struhl, 2003; Venkatesh and Workman, 2015; Xie et al., 2011).

Strikingly, it was found that LTF+ cell lines had widespread loss of histone modifications, including significant loss of histone H3 methylations at K4, K27 and K36 positions as well as acetylations (FIG. 6G), confirming that LTF is associated with severe defects in genic histone remodeling, manifesting as impaired RNAP II function and cryptic transcription. In addition, consistent with widespread epigenetic defects, LTF+ cells also had reduced genome-wide DNA methylation.

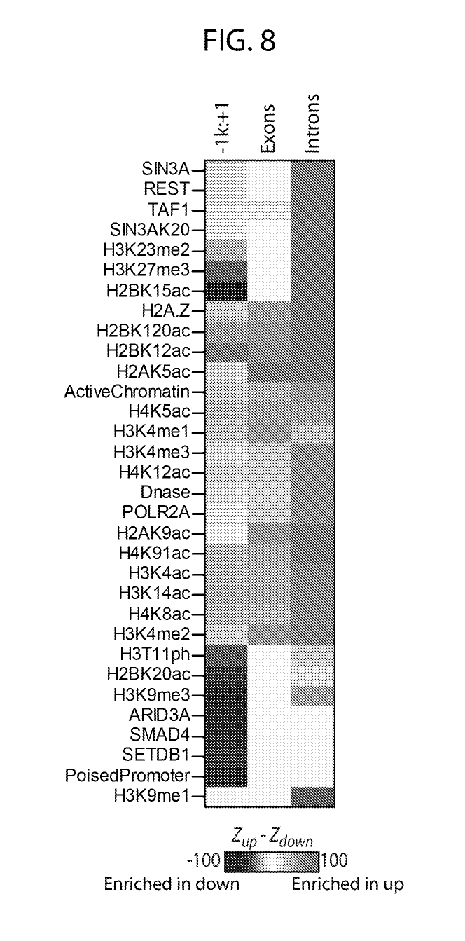

Intriguingly, the Type I and Type II genes (see FIG. 3B) were characterized by markedly distinct chromatin profiles based on the data from Roadmap Epigenome (Chadwick, 2012) (FIG. 8), which can indicate the differential impact of global epigenetic defects on these two classes of genes (see FIG. 8). These results demonstrate that LTF is a phenotype of severe epigenetic, transcription initiation, elongation, capping, mRNA splicing and poly-adenylation defects. Therefore, the 5′-shortening of mRNAs is an expected outcome of poly-A-selected mRNA sequencing of a transcriptome enriched for cryptic unprocessed transcripts, as only the transcripts that were properly terminated would have been captured for sequencing (FIG. 6H).

### Example 5

### LTF+ Tumors have Aberrant Regulation of Long Versus Short Genes

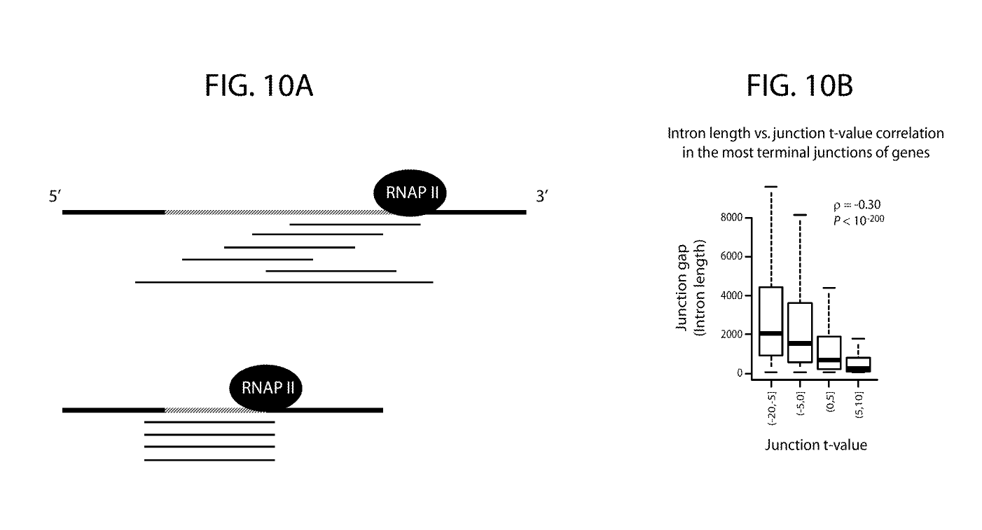

Defective histone remodeling and ensuing impaired transcriptional elongation are expected to have the greatest impact on the transcription of long genes in the genome (Carrozza et al., 2005; Li et al., 2007; Venkatesh and Workman, 2015). Indeed, genes with the most severe shortening and intron retention in LTF (Type I genes, see FIG. 3B) were significantly longer, while those that were overexpressed were among the shortest genes in the genome (FIG. 9A). In addition, exon-exon junctions spanning longer introns were consistently less represented in LTF+ samples, reflecting defective RNAP II elongation along longer DNA segments (FIG. 10).

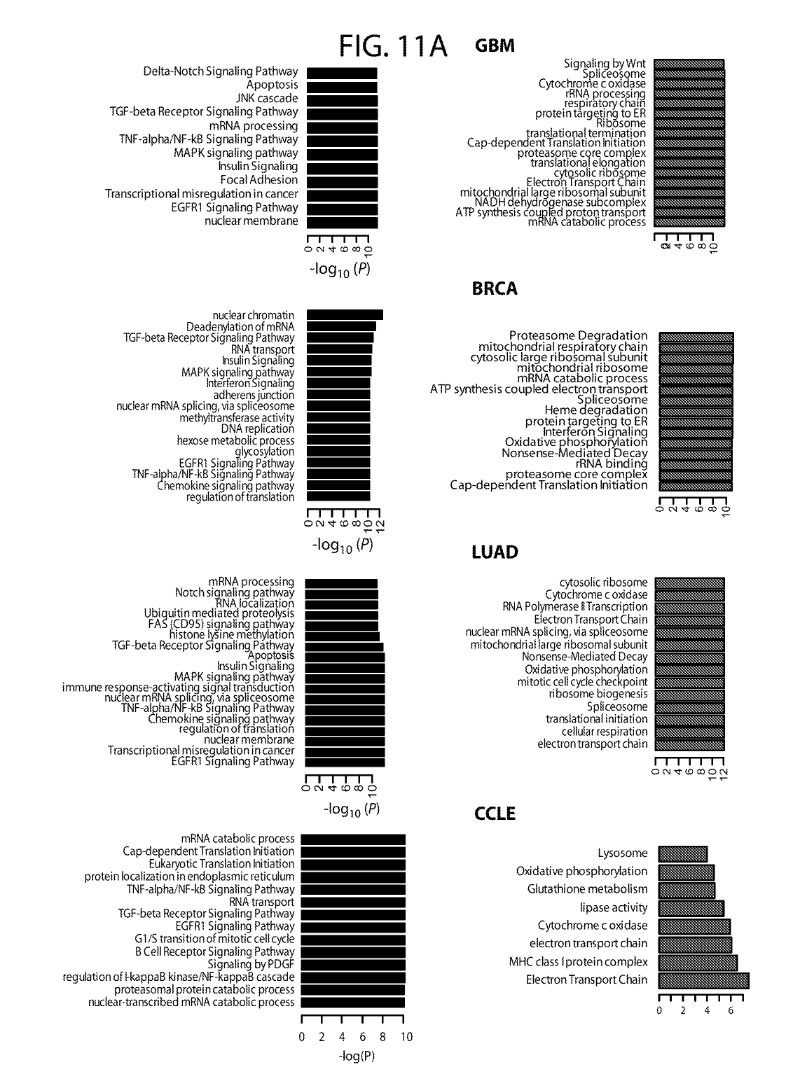

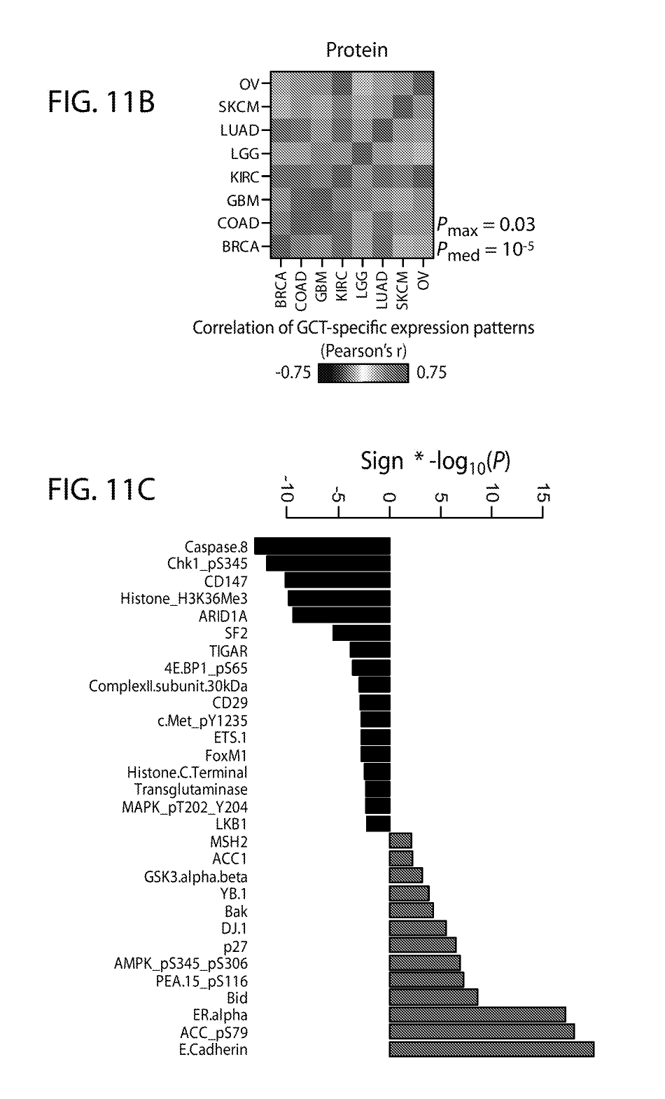

Importantly, pathway enrichment profiles of repressed/shortened (Type I) and overexpressed (Type II) genes in LTF strongly reflect the gene length distributions of their constituent genes (FIG. 9B), where pathways primarily regulated by long genes, such as MAP kinase and immune response signaling, are down-regulated, while those regulated by short genes, such as mitochondrial OXPHOS and ribosome biogenesis, are overexpressed, which was reproduced in LTF+ tumors of many other lineages and in LTF+ cell lines (FIG. 11). The correlation of expression with gene length can also be observed at the protein level; proteins that were consistently repressed in LTF+ tumors had longer gene, mRNA and protein lengths (FIG. 9C-D). Accordingly, LTF+ tumors displayed defective activation of EGFR, MAPK and NF-κB pathways at the protein level, while overexpressing protein synthesis pathway proteins (FIG. 9E-F).

### Example 6

### Some LTF+ Cancers have Mutations in Histone Remodeling Genes

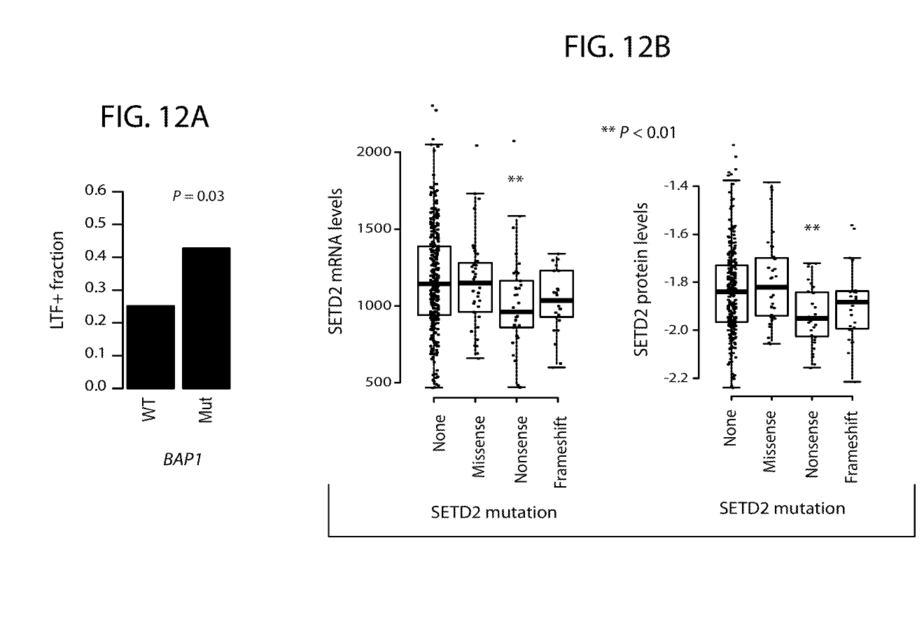

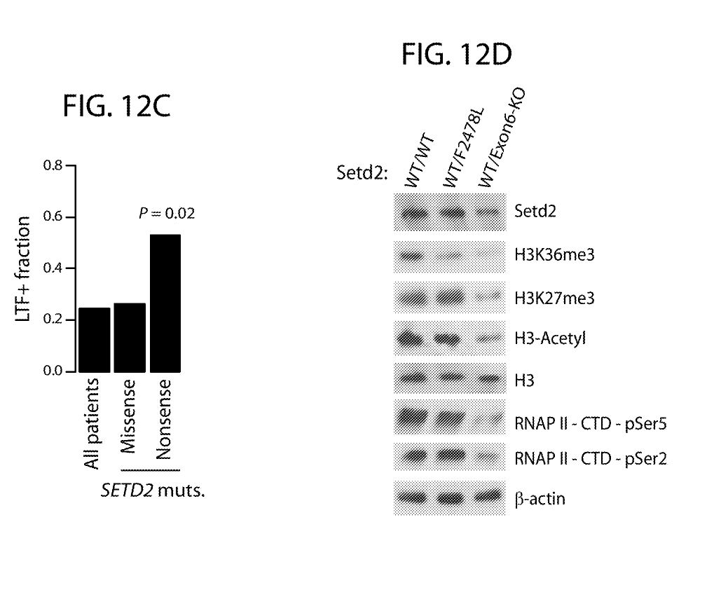

At the genetic level, LTF did not significantly correlate with the most frequent mutations in any of the cancers. However, in clear cell renal cell carcinomas (KIRC), LTF correlated with mutations in BAP1, a histone deubiquitinase involved in DNA damage response and chromatin remodeling, and with nonsense, but not missense, mutations in SETD2, a histone H3 lysine 36 trimethyl-transferase (FIG. 12A-C). The role of gene body histone methylations by SETD2 in suppressing cryptic intragenic transcription has been well-established (Carrozza et al., 2005; Mason and Struhl, 2003; Venkatesh and Workman, 2015), and loss of SETD2 has been shown to lead to widespread spurious intragenic transcription in yeasts (Carrozza et al., 2005), especially in long genes (Li et al., 2007), and in human cells (Carvalho et al., 2013).

Protein-truncating mutations in SETD2, compared to missense mutations, have been reported to have more severe effects on H3K36me3 levels in KIRC tissues, and can lead to widespread mRNA transcription and processing defects (Simon et al., 2014). Accordingly, targeted mutagenesis in the Setd2 gene in mice show that Setd2 nonsense, but not missense, mutations have severe and more widespread effect on histone modifications and RNAP II function (FIG. 12D). Therefore, strong chromatin defects can lead, or predispose, to LTF in cancers. Nevertheless, the mutations in these genes only accounted for less than 15% of LTF+ cases in KIRC, and none of the frequent chromatin modifiers in other cancers correlated with LTF. Therefore, the LTF phenotype is mostly epigenetically, rather than genetically, defined. Accordingly, a significant portion of KIRC tumors with loss of SETD2 expression and H3K36me3 did not have any mutations in SETD2 (Simon et al., 2014).

### Example 7

### LTF Confers Resistance to Immunotherapy in the Clinic

Next, it was determined whether LTF confers worse prognosis to cancer patients. LTF was associated with significantly poor survival only in clear-cell renal cell carcinomas (ccRCC, TCGA code: KIRC). However, stratification of KIRC patients by their therapy modalities reveals that poor prognosis of LTF+ KIRC patients largely reflects their markedly poor response to immunotherapy (primarily with interleukin and interferon (IFN)) compared to LTF− patients (FIG. 13A), although they had a significantly better response to targeted therapy (FIG. 14). Importantly, LTF also correlated with significantly worse outcome in immunotherapy-treated melanoma patients (FIG. 15B), where immunotherapy is also among the primary options (Drake et al., 2014). LTF also predicted worse prognosis in melanoma patients treated with the new immunotherapeutic drugs ilipimumab, nivolumab and pembrolizumab (FIG. 13B), monoclonal antibodies against immune checkpoint pathway inhibitors (Sharma and Allison, 2015), indicating that LTF may confer a generic resistance to anti-tumor immune response.

Next, the correlation of the LTF signature with the clinical response to ipilimumab, a CTLA4 inhibiting antibody, was assessed in the melanoma cohort from Van Allen et al. (Van Allen et al., 2015a), which is the largest published immune checkpoint inhibitor cohort with RNA sequencing data (42 patients). LTF was defined in this cohort as the overall extent of intron retention in Type I genes, as intron retention in Type I genes highly correlated with LTF in TCGA samples (see FIG. 3E-G) (Table 2).

TABLE 2
Intron retention correlating with LTF status.

| | Ratio | |
|---|---|---|
| Patient ID | (Exon_Intron/Exon_Exon) | LTF status |
| Pat02 | 0.07887 | LTF+ |
| Pat03 | 0.07143 | LTF+ |
| Pat04 | 0.06061 | LTF− |
| Pat06 | 0.06329 | LTF− |
| Pat08 | 0.08239 | LTF+ |
| Pat118 | 0.02703 | LTF− |
| Pat119 | 0.04337 | LTF− |
| Pat123 | 0.05357 | LTF− |
| Pat126 | 0.04828 | LTF− |
| Pat14 | 0.06452 | LTF+ |
| Pat15 | 0.06818 | LTF+ |
| Pat16 | 0.07692 | LTF+ |
| Pat19 | 0.06667 | LTF+ |
| Pat20 | 0.07143 | LTF+ |
| Pat25 | 0.08333 | LTF+ |
| Pat27 | 0.06164 | LTF− |
| Pat28 | 0.05988 | LTF− |
| Pat29 | 0.06349 | LTF+ |
| Pat33 | 0.07692 | LTF+ |
| Pat36 | 0.05882 | LTF− |
| Pat37 | 0.04444 | LTF− |
| Pat38 | 0.05422 | LTF− |
| Pat39 | 0.01852 | LTF− |
| Pat40 | 0.07306 | LTF+ |
| Pat41 | 0.125 | LTF+ |
| Pat43 | 0.06 | LTF− |
| Pat44 | 0.05882 | LTF− |
| Pat45 | 0.07692 | LTF+ |
| Pat46 | 0.08485 | LTF+ |
| Pat47 | 0.05556 | LTF− |
| Pat49 | 0.0622 | LTF− |
| Pat50 | 0.07692 | LTF+ |
| Pat79 | 0.0566 | LTF− |
| Pat80 | 0.06667 | LTF+ |
| Pat81 | 0.06667 | LTF+ |
| Pat83 | 0.05707 | LTF− |
| Pat85 | 0.08333 | LTF+ |
| Pat86 | 0.05769 | LTF− |
| Pat88 | 0.06159 | LTF− |
| Pat90 | 0.07634 | LTF+ |
| Pat91 | 0.06452 | LTF+ |
| Pat98 | 0.05714 | LTF− |

Overall intron retention significantly correlated with the non-responding population in this cohort, and predicted worse progression-free (PFS) and overall survival (OS) (FIG. 13C). The LTF-like tumor subgroup (i.e. higher intron retention) in this cohort did not correlate with the total number of non-synonymous mutations (P=0.83, Wilcoxon test), or the expression of cytolytic cell-specific mRNAs, such as GZMA, GZMK and PRF1 (P >0.15, Wilcoxon test), indicating that LTF predicts immunotherapy response independently of tumor mutational burden and tumor infiltration by lymphocytes (TIL), the two factors reported to predict clinical benefits in the original (Van Allen et al., 2015a) and other studies (Tumeh et al., 2014). Importantly, TIL (measured by average expression of GZMK and PRF1) and LTF together had stronger prognostic power in this cohort compared to each alone: LTF− tumors with high TIL did significantly better, while LTF+ and TIL-low tumors did significantly worse (FIG. 13D) (compared with P=0.02 and P=0.09 for OS and PFS, respectively for TIL alone), indicating that the combination of these two variables can be strong predictors of immunotherapy response.

### Example 8

### LTF Impairs Response to Inflammatory Anti-Tumor Cytokines

Resistance to anti-tumor immune responses may be due to immune ignorance to cancer antigens or resistance to immune-mediated anti-tumor attack. For example, many cancers have mutations in the Caspase 8 and 10 genes (CASP8 and CASP10), upstream initiator caspases in the Fas apoptotic pathway used by the cytotoxic T-lymphocytes (CTLs) and Natural Killer cells (NKs) to induce tumor cell death (Abrams, 2005), and these mutations generally correlate with high TIL. Interestingly, LTF+ ccRCC and melanoma samples in TCCA also had higher infiltration by CTLs and NKs compared to LTF− tumors, as judged by the expression of their respective marker genes (GZMA and GZMB, which encode the cytolytic enzymes granzyme A and B) in the bulk tumor samples (FIG. 14B). This indicates that LTF can be an epigenetic mechanism of resistance to immune-mediated anti-tumor attack mechanisms. Indeed, LTF+ tumors display significant repression of the “Fas (CD95) signaling pathway” (FIG. 13E, and see FIG. 9B and FIG. 11A). In addition, PEA-15, a 15 kDa death-effector domain protein encoded by a small gene (˜10 kb), and a negative regulator of the Fas apoptotic pathway (Condorelli et al., 1999), was one of the most consistently overexpressed proteins in LTF+ tumors (see FIG. 9E) and cell lines (see FIG. 11C).

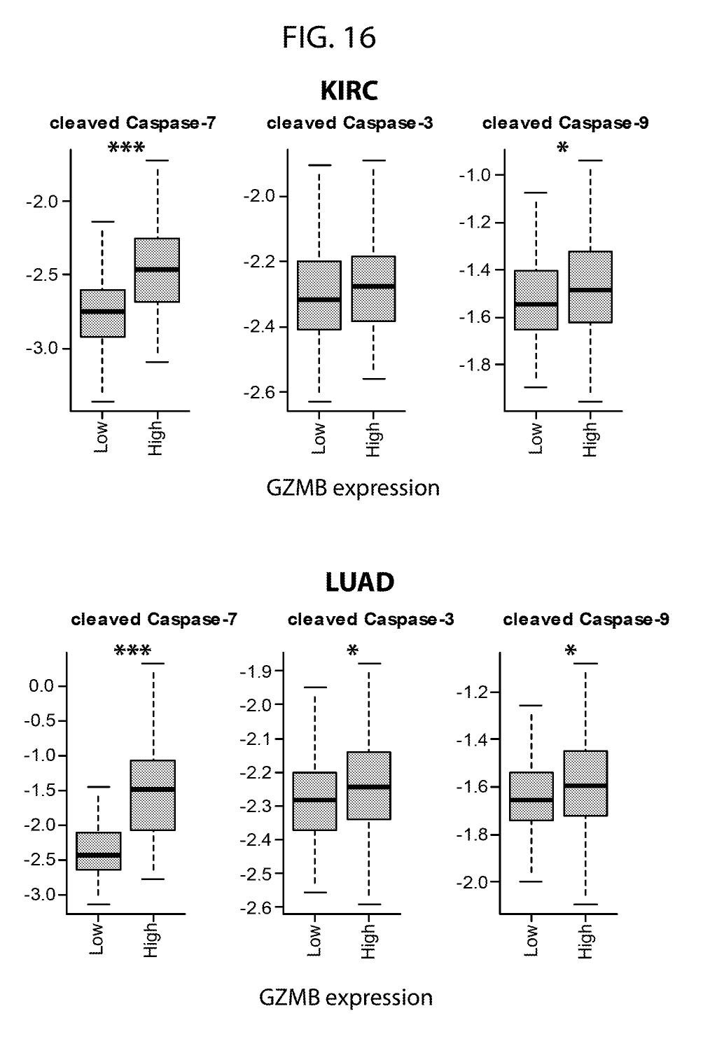

To test whether LTF correlates with reduced TIL-mediated tumor cytolytic activity in patient samples, the correlation of LTF with the levels of cleaved (i.e. active) Caspase 7 (measured by RPPA) in KIRC and SKCM tumor samples was measured. Caspase 7 cleavage is a major milestone in both FasL and granzyme-mediated cell death, and, importantly, cleaved Caspase 7 was the only caspase protein that strongly correlated with immune infiltration in different cancers, indicating that Caspase 7 cleavage reflects TIL-mediated tumor cell killing (FIG. 16). Importantly, LTF+ tumors characterized by high TIL (measured by GZMB expression) had significantly less cleaved Caspase 7 relative to LTF− cells (FIG. 13F), indicating that LTF suppresses TIL-mediated tumor killing. Accordingly, LTF+ cell lines had reduced expression and activity of Caspase 8, and were more resistant to cell killing induced by FasL in vitro (FIG. 13G-H, and see FIG. 11C for Caspase 8 levels in LTF+ cell lines based on published RPPA data).

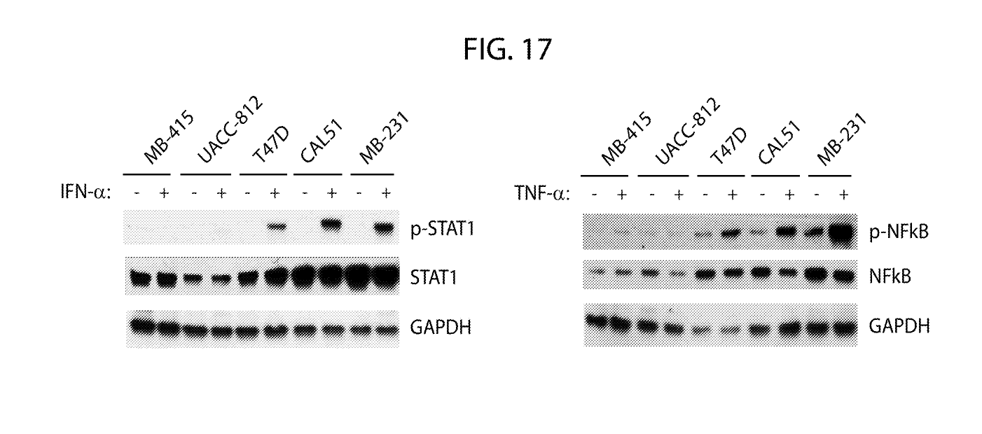

In addition to the Fas pathway, the Type I genes include multiple inflammatory pathway genes; and the levels of total or activated NF-κB, STAT3 and STATS proteins are consistently reduced in LTF+ cancers (see FIG. 9E-F). Interferon signaling through STAT1 in the resident tumor cells was found to highly correlate with immunotherapy response (Tumeh et al., 2014), and tumor cell-intrinsic interferon and NF-κB signaling have been found to be required for the priming of tumor cells for CTL-mediated killing (Ahn et al., 2002; Bald et al., 2014; Liu et al., 2012; Wigginton et al., 2001), indicating that impaired inflammatory response signaling in LTF+ tumors can also contribute to immunotherapy resistance. Consistent with this, LTF+ cell lines had reduced expression of several inflammatory response proteins, and, importantly, were defective in their response to IFN and TNF-α (FIG. 17 Supp.FIG. 11).

### Example 9

### Disruption of Epigenetic and Transcriptional Functions Confers Resistance to Anti-Tumor Immunity

The present observations show that LTF correlates with defective inflammatory response phenotype on cancer cells, conferring escape from anti-tumor immunity. Next, it was determined whether the disruption of gene body histone remodeling and transcriptional elongation is sufficient to impair the transcription of inflammatory response genes, and dampen response to immune-mediated anti-tumor insults. To test this, SETD2 expression was stably silenced in LTF− breast cancer cell lines T47D and CAL51, as SETD2 loss has been shown to lead to LTF-like transcriptional defects and, furthermore, it correlates with LTF in KIRC (see FIG. 12). Intriguingly, SETD2 knock-down led to widespread reduction of histone modifications in addition to H3K36me3, including acetylations of H3, and trimethylations at K4 and K27 (FIG. 15A). In addition, SETD2 ablation led to significant reduction in total RNAP II levels, and in its Ser5 and Ser2 phosphorylations (FIG. 15A), consistent with LTF (see FIG. 6) and Setd2 knock-out in mouse cells (see FIG. 12D). Also consistent with LTF and impaired RNAP II function, SETD2 silencing led to significant defects in mRNA capping and poly-adenylation (FIG. 15B-E). Importantly, SETD2-silenced cells had reduced expression of multiple inflammatory pathway proteins, impaired response to pro-inflammatory stimuli (FIG. 15D) and significant resistance to FasL-mediated cell death (FIG. 15E-F).

To test if the direct inhibition of RNAP II elongation can cause a similar effect, cells were treated with the sublethal doses of flavopiridol, a CDK9 (kinase component of p-TEFb) inhibitor. Intriguingly, prolonged inhibition of RNAP II Ser2 phosphorylation by CDK9 mimicked both LTF and SETD2 silencing in terms of widespread epigenetic and transcriptional defects, and resistance to pro-inflammatory stimuli and FasL challenge (FIG. 15G-I). In order to test if flavopiridol can confer escape from anti-tumor immune attack in vivo, the effect of prolonged sublethal flavopiridol treatment on the ability of B16/F10 mouse melanoma cells to escape from NK-mediated tumor rejection was tested. Seeding of B16/F10 cells in the lungs of C57BL6 mice following tail vein injection has been shown to be highly sensitive to NK cell function (Shehata et al., 2015). Intriguingly, CDK9 inhibition significantly increased the ability of B16 cells for lung seeding compared to control cells, whereas the effect was diminished in NK-depleted mice (FIG. 15J).

These results strongly indicate that 1) the gene body histone remodeling and RNAP II functions are highly inter-dependent, 2) they are required to maintain immune response competence of cells, and 3) their perturbation in cancer cells can confer resistance to immune-mediated anti-tumor attacks.

### Example 10

### LTF Impairs Response to Inflammatory Anti-Tumor Cytokines

A sample having tumor cells is obtained from a patient having cancer, or one or more symptoms thereof. The sample is analyzed, by RNA and/or protein analysis to determine whether the tumor cells have a loss of transcriptional fidelity (LTF) phenotype. The LTF phenotype is characterized by: having a preferential expression or higher proportion of one or more aberrant or non-canonical mRNA isoforms, relative to a control value for expression or proportion; and/or by reduced expression or reduced presence of one or more proteins selected from the group consisting of RNAP II Ser2, RNAP II Ser5, H3K4me3, H3K27me3, and H3K36me3 relative to a respective control value of expression or presence of RNAP II Ser2, RNAP II Ser5, H3K4me3, H3K27me3, and H3K36me3. The LTF phenotype can also be evaluated on the basis of presence of severe epigenetic, transcription initiation, elongation, capping, mRNA splicing and poly-adenylation defects.

The patient is then treated based on a lack of suitability of immunotherapy where the tumor cells of the subject have an LTF phenotype, or determining a suitability of immunotherapy where the tumor cells of the subject lack an LTF phenotype. Where the patient has the LTF phenotype, the patient is administered or assigned a treatment which does not include immunotherapy, but which does include at least one of chemotherapy and/or targeted therapy and/or alternative therapy, provided that the targeted therapy is not an immunotherapy. Where the patient lacks the LTF phenotype, the patient is administered or assigned a treatment which includes immunotherapy.

The methods and materials used in the above-described experiments are described below.

Cells and Reagents:

UACC-812 and MDA-MB-415 cells were purchased from ATCC (Manassas, Va.). UACC-812 cells were grown in Leibovitz's L-15 (Gibco) medium with 2 mM L-glutamine containing 20% fetal bovine serum (FBS) and 0.1% antibiotic and antimycotic (Gibco). MDA-MB-415 cells were grown in Leibovitz's L-15 (Gibco) medium with 2 mM L-glutamine supplemented with 10 μg/ml insulin (Sigma), 10 μg/ml glutathione (Calbiochem), 15% FBS and 0.1% antibiotic and antimycotic (Gibco). SKBR3, BT474, MDA-MB-231, CAL51, T47D cells were cultured in RPMI 1640 (Gibco) containing 10% FBS with 0.1% antibiotic and antimycotic (Gibco). MDA-MB-453 cells were cultured in improved minimum essential medium (Gibco) containing 20% FBS with 0.1% antibiotic and antimycotic (Gibco). All cells were cultured in a humidified atmosphere in 5% CO2 at 37° C.

Immunoblotting:

Total proteins were extracted with RIPA buffer (Santa Cruz Biotechnology, sc-24948), and 15 μg protein from each sample was run in a 4-18% SDS polyacrylamide gel (Bio-Rad), and transferred onto polyvinylidene difluoride membranes. The membranes were blocked in 5% dry milk in tris-buffered saline—Tween 20 for 1 hour. Blocked membranes were incubated overnight with primary antibodies against pSer5-RNA polymerase II (1:1000, Active motif), pSer2-RNA polymerase II (1:1000, Active motif), RNA polymerase II (1:1000, Active motif), SETD2 (1:1000, abcam), CyclinT1 (1:1000, Santa Cruz), H3K36me3 (1:5000, abcam), H3K27me3 (1:5000, Active motif), Pan-acetyl-H3 (1:5000, Cell Signaling), Histone H3 (1:5000, Cell Signaling), pMAPK (1;1000, Cell Signaling), MAPK (1;1000, Cell Signaling), pAKT (1:1000, Cell Signaling), STAT1 (1:1000, Cell Signaling), pSTAT1 (1:1000, Cell Signaling), NF-κB (1:1000, Cell Signaling), pNF-κB (1:1000), Cleaved-PARP(1:1000, Cell Signaling), Caspase-3 (1;1000, Cell Signaling), β-Actin (1;1000, Cell Signaling), GAPDH (1:1000, Cell Signaling) in 5% bovine serum albumin. After washing and incubating with the appropriate secondary antibody (anti-rabbit IgG or anti-Rat IgG (1:5000, Cell signaling)), protein signals were detected with enhanced chemiluminescence (Millipore).

Cytokine Treatments:

Equal numbers of cells (10⁵) cells were seeded into 12 well culture plates in their corresponding growth medium. Next day, cells were treated with IFN-α (5 ng/ml) or TNF-α (5 ng/ml) for 45 minutes and protein was extracted in RIPA buffer.

PolyA Tail mRNA Capture:

Total RNA was extracted from the cells using Tri reagent (Sigma), followed by rRNA depletion and subsequent concentration of rRNA-depleted samples using RiboMinus™ Eukaryote Kit (Ambion) according to manufacturer's instructions. PolyA+-RNA was isolated from rRNA-depleted samples using Dynabeads® Oligo(dT)25 (Ambion) according to the manufacturer's instructions. Purity and concentration of RNA yield were measured by NanoDrop (Thermo Scientific). The 260/280 ratio was 1.90-2.00, and the 260/230 ratio was 2.00-2.20 for all RNA Samples.

5′ Capped RNA IMMUNOPRECIPITATION:

Five-prime capped RNAs were immunoprecipitated with the monoclonal 7-Methylguanosine antibody (BioVision) coated protein A columns, from total RNA devoid of rRNA using RiboMinus™ Eukaryote Kit (Ambion) according to manufacturer's instructions. Purity and concentration of RNA yield were measured by NanoDrop (Thermo Scientific). The 260/280 ratio was 1.90-2.00, and the 260/230 ratio was 2.00-2.20 for all RNA Samples.

Cytotoxicity Assay:

Equal number of cells was seeded into the wells of 96-well culture plates in their corresponding medium and incubated overnight in a 5% CO2 humidified incubator. Cells were then treated with different concentrations of hhis6FasL (0.1 ng/ml-1000 ng/ml) in the presence of 10 μg/ml anti-His antibody (Cell Signaling) for 24 hours. Dead cells were removed by washing with PBS buffer and the attached cells were fixed and stained with crystal violet solution [20% methanol, 0.5% crystal violet (Sigma) in 1× phosphate-buffered saline (PBS)] for 30 min. Excess stain was removed by gently rinsing the plates in tap water, and the plates were dried at room temperature. Crystal violet crystals were redissolved in Triton (Amresco), and cell density was determined by measuring the absorbance at 570 nm in a microplate reader (Bio-Tek Instruments).

Caspase 8 Activity Assay:

Equal number of cells (105) were seeded into 96-well plates, and treated with hhis6FasL (long/mL) in the presence of 10 μg/ml anti-His antibody. Caspase 8 activity was assessed after 6 hours using colorimetric Caspase 8 assay kit (Abcam ab39700) according to manufacturer protocol. The absorbance was measured at 400 nm using the microplate reader (Bio-Tek Instruments).

RNA Isolation:

Total RNAs were extracted from the cells using Tri reagent (Sigma). RNase-free DNase was used for removing all genomic DNA contamination. The RNA was precipitated by Isopropanol (Sigma), washed by ice cold 75% ethanol (Sigma), and air dried prior to resuspension in 20 μl of DEPCtreated water. Purity and concentration of RNA was measured by NanoDrops (Thermo Scientific). The 260/280 ratio was 1.90-2.00 and the 260/230 ratio was 2.00-2.20 for all RNA Samples.

Sequencing:

RNA-seq was performed by Genomics, Epigenomics and Sequencing Core (GESC) in the University of Cincinnati. Using PrepX mRNA Library kit (WaferGen) and Apollo 324 NGS automatic library prep system, the isolated RNA was RNase III fragmented, adaptor-ligated and Superscript III reverse transcriptase (Lifetech, Grand Island, N.Y.) converted into cDNA, followed by automatic purification using Agencourt AMPure XP beads (Beckman Coulter, Indianapolis Ind.). The targeted cDNA fragment is around 200 bp. Indexed libraries were proportionally pooled (20-50 million reads per sample in general) for clustering in cBot system (Illumina, San Diego, Calif.). Libraries at the final concentration of 15.0 pM was clustered onto a single read (SR) flow cell using Illumina's TruSeq SR Cluster kit v3, and sequenced for 50 bp using TruSeq SBS kit on Illumina HiSeq system.

Data Processing:

All RNA-seq data were prepared using a slightly modified UNC RNA-seq pipeline v2. Briefly, single- (for the RNAseq data) or paired-end (TCGA and CCLE) FASTQ files were formatted using UNC-Chapel Hill Bioinformatics Utilities (ubu v1.2, https <colon slash slash> github <dot> corn <slash> mozack <slash> ubu) and aligned against reference genome (hg19) using MapSplice (v2.1.9) (Wang et al., 2010). Resulting BAM files were sorted by chromosome, then translated to transcriptome coordinates using ubu package. Indels, large inserts (max=10,000), zero mapping quality reads were all filtered out from the transcriptome BAM files.

Transcript quantification from these filtered BAMs were done using RSEM (v1.2.20) (Li and Dewey, 2011). After stripping trailing tabs from isoform quantification files, isoforms were pruned from gene quantification files. Normalized gene and isoform counts were calculated from raw counts divided by the 75-percentile and then multiplied by 1000. Junction and exon/intron quantifications were calculated using ubu package and coverageBed (BedTools v2.17.0, http <colon slash slash> bedtools <dot> readthedocs <dot> org <slash> en<slash> latest), respectively.

Differential Exon Expression Heatmap:

For exon-level heatmap in FIG. 6A, junction and intron analyses, the t-statistic of difference (t-value) in the expression of each exon, junction and intron was calculated between LTF+ and LTF− tumor samples. Every “expressed” gene (i.e. has a 90%-ile value of >30 in a given cancer (e.g. KIRC) dataset) was defined by 20 exon (or junction) bins (genes with <20 exons (or junctions) were stretched, and those >20 exons were compressed, into 20 bins), and corresponding exon (or junction) t-values were visualized in a heatmap where columns (bins) were ordered from 5′ to 3′. For exon and junction analyses, pre-computed RPKM and raw read values, respectively, were used as provided in TCGA data matrix. Intron RPKM values were obtained from analyses of the mRNA-seq FASTQ files for 9 LTF− and 7 LTF+ KIRC samples from TCGA using the UCSC definition for introns.

Intron to exon expression ratios were calculated for each gene by taking the ratio of total intron expression (sum of all intron RPKM values) to that of exon expression.

Intron Retention Analyses:

RNAseq reads were mapped using TopHat (Trapnell et al., 2010). The barn files were then processed using custom python script using the pysam library to extract read counts of exon-exon junctions and exon-intron junctions. Briefly: for each gene, reads were extracted from the genomic regions defined by the start and stop site. Split reads with 8 bp anchors (a minimum of 8 bp mapped to each exon) and read mapping quality >20 were extracted and the junction was annotated by the start and stop positions of the gap. The number of reads mapping to each exon-exon junction was counted. For evert exon-exon junction, identified reads+/−150 bp around the exon-intron and intron-exon junctions were extracted, and the expression of these junctions was counted as the number of reads that span across the exon-intron/intron-exon junction with read mapping quality >20 and at least 8 bp on each corresponding exon and intron. For the ratio analyses of exon-intron and exon-exon junction reads, only exon-exon junctions with at least 5 mapped reads and the intron length >500 bp were used. Using different cutoffs for either of these parameters did not significantly affect the results.

Datasets:

All processed RNAseq, somatic mutations and clinical data were obtained from TCGA data portal. The raw RNAseq data (FASTQ files) from TCGA (with authorization) and Cancer Cell Line Encyclopedia (public) were obtained from the Cancer Genomics Hub (http <colon slash slash> cghub <dot> ucsc <dot> edu). RPPA data for breast cancer cell lines was obtained from the TCPA (Li et al., 2013) web site (http <colon slash slash> bioinformatics <dot> mdanderson <dot> org <slash> main <slash> Public Datasets). For RNAseq data, normalized count values were used for all gene and isoform analyses. RPKM values were used for exon-level analyses, and raw read numbers were used for junction analyses. Gene-to-isoform and gene-to-exon mappings were obtained from TCGA gaf file.

Gene, mRNA and Protein Lengths:

Gene and mRNA lengths were obtained from UCSC genome browser. Protein lengths were obtained from Human Protein Reference Database. Relative protein lengths were obtained by dividing the length of each mRNA or protein isoform by that of the longest isoform of the corresponding gene. Relative isoform expression in the heatmap in FIG. 3E was calculated by dividing the expression value of an isoform by sum of all isoforms for its corresponding gene.

Modified Pearson's Correlation:

In correlation of t-values to each other, majority of values usually lie in the “non-significant” (i.e. absolute value <2) region of the t distribution. These values are likely to contribute to “noise” in the correlation analyses of two t-value distributions. Therefore, correlating two t-value distributions only considered cases that had |t|>2 in either of the two samples being analyzed (i.e. cases with <2 in both samples are discarded from correlation analysis).

ChIP-Seq Data Analyses in FIG. 11:

Different regions of the gene bodies of gene sets that were repressed (Type I genes in FIG. 9) or overexpressed (Type II genes in FIG. 9) in LTF+ tumors were analyzed for overlap with a large collection of genome-wide functional genomics datasets. First, data relevant to gene regulation from a variety of sources was compiled, including ENCODE (Consortium, 2012), Roadmap Epigenomics (Roadmap Epigenomics et al., 2015), the UCSC Genome Browser (Kent et al., 2002), and Pazar (Portales-Casamar et al., 2009). For both gene sets, the constitutive genes were broken into different regions, and these regions were overlapped with each of the 2,345 functional genomics datasets. Three regions were considered in total: (−1,000,+1) relative to the transcription start site (TSS) (promoter), all exons and all introns.

To illustrate, consider the promoter regions of the Type I gene set. For each gene in the set, the genomic coordinates of its promoter were looked up, and these coordinates were then intersected with each of the 2,345 datasets. The observed overlap between the set of promoters and a given dataset were then calculated as the number of promoters that overlap that dataset by at least one base. Next it was determined how significantly different the observed overlap was from the expected overlap with each dataset. To do so, a matched random set of promoters was created. For each gene in the Type I set, a gene was randomly picked from the background set of 10,448 expressed genes (from the heatmap in FIG. 9D), and a simulated promoter was generated by matching the promoter length of the corresponding gene in the Type I set. This procedure therefore guarantees that the promoter length distribution of the random set will match the real set. For this random set, the overlap with each dataset was then calculated. This procedure was repeated 1,000 times, resulting in a distribution of expected overlaps between the promoters and each dataset that follows a normal distribution, which was used to generate a Z-score and P-value for the observed number of overlaps. For example, if 50/100 promoters overlapped peaks from a given ChIP-seq dataset, and 10+/−5 was expected, this yields a Z-score of 8. This procedure was repeated for each of the 3 gene regions listed above. To compare between the Type I and II gene sets, delta values were calculated based on the difference between the two Z-scores. This resulted in a list of genomic features specific to the gene regions of the “up” set relative to the “down” set, and vice versa.

Setd2-Mutant Mice:

The CRISPR-cas9 technology was used to generate the point mutation F2478L, which is equivalent of SETD2-F2505L mutation found in an AML patient (Zhu et al., 2014). Setd2-F2478L mutation is in the SRI domain, causes complete loss of the interaction with the C-terminal domain (CTD) of RNA pol II (Li et al., 2005). The SRI domain in SETD2, along with the catalytic SET domain, is frequently mutated in human cancers. The same CRISPR-cas9 technology was used to generate the Setd2-Exon6 KO/WT mice, which has a deletion of exon 6 of Setd2 mediated by NHEJ after cut by two guide RNAs (gRNAs). This resulted in a frameshift in the middle of the SET methyltransferase domain and nonsense mediated decay of mRNA. Both alleles were validated by TA cloning and sequencing of genomic DNA (YD and GH, in preparation).

Survival Analyses:

Clinical survival data were obtained from TCGA. Patient stratification was done by classifying patients into non-exclusive lists based on drugs they received. Since drug annotations were not consistent (i.e. the same drug was annotated with different spellings for different patients), a vocabulary of immunotherapy drug annotations in the TCGA clinical samples for SKCM and KIRC was compiled. For immunotherapy drugs, our vocabulary included Alferon, GM-CSF, IL-18, IL-2, IL2, interferon, Interferon, Interferon-?2, Interferon-alfa, Interferon alfa, Interferon alfa-2b, interferon alpha, Interferon alpha, Interferon Alpha, Interleukin-2, Interleukin-2, Laferon, Leukine, Alpha Interferon, IFN-Alpha (Intron), IL-2 (high dose), IL-2 Thearpy (interleukin), INF, interferon-alpha, interleukin-2, Interleukin 2-high dose, Intron A, Proleukin, proleukin (IL-2), Imiquimod, Sylatron, Resiquimod and Diphencyprone. For checkpoint inhibitor therapy, ipilimumab, Yervoy, pembrolizumab, Pembrolizumab and Ipilimumab annotations were considered.

Analyses of the Van Allen Cohort:

The LTF-like phenotype in this cohort was defined as increased global retention of exon-junction reads in Type I genes, same as in FIG. 9E-G. LTF+ and LTF− populations were defined by a cutoff at the median (see Table 2 for LTF assignments) (21 samples each). To score TIL, the average expressions of GZMK and PRF1 (perforin) were used, and TIL-high and TIL-low populations were again determined by a cutoff at the median. Using just GZMK instead of the average, or just PRF1, or GZMA instead of GZMK, in lieu of TIL gave similar results.

Testing NK-Mediated Tumor Cell Killing In Vivo:

C57Bl/6 mice were injected with control or flavopiridol (100 μM) treated 2×105 B16-OVA cells into tail veins. One hour later, the lungs were harvested, digested in liberase and the frequency of tumor cells was assessed using quantitative PCR (Shehata et al., 2015). mRNA levels for OVA (B16-OVA) were assessed and normalized to GAPDH. To demonstrate that the observed effect is NK cell dependent, parallel groups were treated with NK depleting agent anti-asialo GM1 (20 ul, 24 hr before the start of the experiment). Six mice for each group were used. The protocol and use of mice were performed with the approval of the Cincinnati Children's Institutional Animal Care and Use Committee.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the invention. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

Abrams, S. I. (2005). Positive and negative consequences of Fas/Fas ligand interactions in the antitumor response. Front Biosci 10, 809-821.
Ahn, E. Y., Pan, G., Vickers, S. M., and McDonald, J. M. (2002). IFN-gammaupregulates apoptosis-related molecules and enhances Fas-mediated apoptosis in human cholangiocarcinoma. Int J Cancer 100, 445-451.
Bald, T., Landsberg, J., Lopez-Ramos, D., Renn, M., Glodde, N., Jansen, P., Gaffal, E., Steitz, J., Tolba, R., Kalinke, U., et al. (2014). Immune cell-poor melanomas benefit from PD-1 blockade after targeted type I IFN activation. Cancer Discov 4, 674-687.
Carrozza, M. J., Li, B., Florens, L., Suganuma, T., Swanson, S. K., Lee, K. K., Shia, W. J., Anderson, S., Yates, J., Washburn, M. P., et al. (2005). Histone H3 methylation by Set2 directs deacetylation of coding regions by Rpd3S to suppress spurious intragenic transcription. Cell 123, 581-592.
Carvalho, S., Raposo, A. C., Martins, F. B., Grosso, A. R., Sridhara, S. C., Rino, J., Carmo-Fonseca, M., and de Almeida, S. F. (2013). Histone methyltransferase SETD2 coordinates FACT recruitment with nucleosome dynamics during transcription. Nucleic acids research 41, 2881-2893.
Chadwick, L. H. (2012). The NIH Roadmap Epigenomics Program data resource. Epigenomics 4, 317-324.
Cheung, V., Chua, G., Batada, N. N., Landry, C. R., Michnick, S. W., Hughes, T. R., and Winston, F. (2008). Chromatin- and transcription-related factors repress transcription from within coding regions throughout the Saccharomyces cerevisiae genome. PLoS biology 6, e277.
Condorelli, G., Vigliotta, G., Cafieri, A., Trencia, A., Andalo, P., Oriente, F., Miele, C., Caruso, M., Formisano, P., and Beguinot, F. (1999). PED/PEA-15: an anti-apoptotic molecule that regulates FAS/TNFR1-induced apoptosis. Oncogene 18, 4409-4415.
Consortium, E. P. (2012). An integrated encyclopedia of DNA elements in the human genome. Nature 489, 57-74.
Drake, C. G., Lipson, E. J., and Brahmer, J. R. (2014). Breathing new life into immunotherapy: review of melanoma, lung and kidney cancer. Nature reviews Clinical oncology 11, 24-37.
Dvinge, H., and Bradley, R. K. (2015). Widespread intron retention diversifies most cancer transcriptomes. Genome Med 7, 45.
Ho, C. K., and Shuman, S. (1999). Distinct roles for CTD Ser-2 and Ser-5 phosphorylation in the recruitment and allosteric activation of mammalian mRNA capping enzyme. Molecular cell 3, 405-411.
Jonkers, I., and Lis, J. T. (2015). Getting up to speed with transcription elongation by RNA polymerase II. Nature reviews Molecular cell biology 16, 167-177.
Kanu, N., Gronroos, E., Martinez, P., Burrell, R. A., Yi Goh, X., Bartkova, J., Maya-Mendoza, A., Mistrik, M., Rowan, A. J., Patel, H., et al. (2015). SETD2 loss-of-function promotes renal cancer branched evolution through replication stress and impaired DNA repair. Oncogene 34, 5699-5708.
Kaplan, C. D., Laprade, L., and Winston, F. (2003). Transcription elongation factors repress transcription initiation from cryptic sites. Science 301, 1096-1099.
Kent, W. J., Sugnet, C. W., Furey, T. S., Roskin, K. M., Pringle, T. H., Zahler, A. M., and Haussler, D. (2002). The human genome browser at UCSC. Genome research 12, 996-1006.
Li, B., and Dewey, C. N. (2011). RSEM: accurate transcript quantification from RNA-Seq data with or without a reference genome. BMC bioinformatics 12, 323.
Li, B., Gogol, M., Carey, M., Pattenden, S. G., Seidel, C., and Workman, J. L. (2007). Infrequently transcribed long genes depend on the Set2/Rpd3S pathway for accurate transcription. Genes & development 21, 1422-1430.
Li, J., Lu, Y., Akbani, R., Ju, Z., Roebuck, P. L., Liu, W., Yang, J. Y., Broom, B. M., Verhaak, R. G., Kane, D. W., et al. (2013). TCPA: a resource for cancer functional proteomics data. Nature methods 10, 1046-1047.
Li, M., Phatnani, H. P., Guan, Z., Sage, H., Greenleaf, A. L., and Zhou, P. (2005). Solution structure of the Set2-Rpb1 interacting domain of human Set2 and its interaction with the hyperphosphorylated C-terminal domain of Rpb1. Proc Natl Acad Sci USA 102, 17636-17641.
Liu, F., Bardhan, K., Yang, D., Thangaraju, M., Ganapathy, V., Waller, J. L., Liles, G. B., Lee, J. R., and Liu, K. (2012). NF-kappaB directly regulates Fas transcription to modulate Fas-mediated apoptosis and tumor suppression. J Biol Chem 287, 25530-25540.
Luco, R. F., Pan, Q., Tominaga, K., Blencowe, B. J., Pereira-Smith, O. M., and Misteli, T. (2010). Regulation of alternative splicing by histone modifications. Science 327, 996-1000.
Mason, P. B., and Struhl, K. (2003). The FACT complex travels with elongating RNA polymerase II and is important for the fidelity of transcriptional initiation in vivo. Molecular and cellular biology 23, 8323-8333.
Mayr, C., and Bartel, D. P. (2009). Widespread shortening of 3′UTRs by alternative cleavage and polyadenylation activates oncogenes in cancer cells. Cell 138, 673-684.
Nilson, K. A., Guo, J., Turek, M. E., Brogie, J. E., Delaney, E., Luse, D. S., and Price, D. H. (2015). THZ1 Reveals Roles for Cdk7 in Co-transcriptional Capping and Pausing. Molecular cell 59, 576-587.
Pfister, S. X., Ahrabi, S., Zalmas, L. P., Sarkar, S., Aymard, F., Bachrati, C. Z., Helleday, T., Legube, G., La Thangue, N. B., Porter, A. C., et al. (2014). SETD2-dependent histone H3K36 trimethylation is required for homologous recombination repair and genome stability. Cell Rep 7, 2006-2018.
Plass, C., Pfister, S. M., Lindroth, A. M., Bogatyrova, O., Claus, R., and Lichter, P. (2013). Mutations in regulators of the epigenome and their connections to global chromatin patterns in cancer. Nat Rev Genet 14, 765-780.
Portales-Casamar, E., Arenillas, D., Lim, J., Swanson, M. I., Jiang, S., McCallum, A., Kirov, S., and Wasserman, W. W. (2009). The PAZAR database of gene regulatory information coupled to the ORCA toolkit for the study of regulatory sequences. Nucleic acids research 37, D54-60.
Roadmap Epigenomics, C., Kundaje, A., Meuleman, W., Ernst, J., Bilenky, M., Yen, A., Heravi-Moussavi, A., Kheradpour, P., Zhang, Z., Wang, J., et al. (2015). Integrative analysis of 111 reference human epigenomes. Nature 518, 317-330.
Sharma, P., and Allison, J. P. (2015). Immune checkpoint targeting in cancer therapy: toward combination strategies with curative potential. Cell 161, 205-214.
Shehata, H. M., Hoebe, K., and Chougnet, C. A. (2015). The aged nonhematopoietic environment impairs natural killer cell maturation and function. Aging Cell 14, 191-199.
Simon, J. M., Hacker, K. E., Singh, D., Brannon, A. R., Parker, J. S., Weiser, M., Ho, T. H., Kuan, P. F., Jonasch, E., Furey, T. S., et al. (2014). Variation in chromatin accessibility in human kidney cancer links H3K36 methyltransferase loss with widespread RNA processing defects. Genome research 24, 241-250.
Sowalsky, A. G., Xia, Z., Wang, L., Zhao, H., Chen, S., Bubley, G. J., Balk, S. P., and Li, W. (2015). Whole transcriptome sequencing reveals extensive unspliced mRNA in metastatic castration-resistant prostate cancer. Mol Cancer Res 13, 98-106.
Thorvaldsdottir, H., Robinson, J. T., and Mesirov, J. P. (2013). Integrative Genomics Viewer (IGV): high-performance genomics data visualization and exploration. Brief Bioinform 14, 178-192.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nat Biotechnol 28, 511-515.
Tumeh, P. C., Harview, C. L., Yearley, J. H., Shintaku, I. P., Taylor, E. J., Robert, L., Chmielowski, B., Spasic, M., Henry, G., Ciobanu, V., et al. (2014). PD-1 blockade induces responses by inhibiting adaptive immune resistance. Nature 515, 568-571.
Van Allen, E. M., Miao, D., Schilling, B., Shukla, S. A., Blank, C., Zimmer, L., Sucker, A., Hillen, U., Foppen, M. H., Goldinger, S. M., et al. (2015a). Genomic correlates of response to CTLA-4 blockade in metastatic melanoma. Science 350, 207-211.
Van Allen, E. M., Miao, D., Schilling, B., Shukla, S. A., Blank, C., Zimmer, L., Sucker, A., Hillen, U., Geukes Foppen, M. H., Goldinger, S. M., et al. (2015b). Genomic correlates of response to CTLA-4 blockade in metastatic melanoma. Science 350, 207-211.
Venkatesh, S., and Workman, J. L. (2015). Histone exchange, chromatin structure and the regulation of transcription. Nature reviews Molecular cell biology 16, 178-189.
Wang, K., Singh, D., Zeng, Z., Coleman, S. J., Huang, Y., Savich, G. L., He, X., Mieczkowski, P., Grimm, S. A., Perou, C. M., et al. (2010). MapSplice: accurate mapping of RNA-seq reads for splice junction discovery. Nucleic acids research 38, e178.
Watson, I. R., Takahashi, K., Futreal, P. A., and Chin, L. (2013). Emerging patterns of somatic mutations in cancer. Nat Rev Genet 14, 703-718.
Wigginton, J. M., Gruys, E., Geiselhart, L., Subleski, J., Komschlies, K. L., Park, J. W., Wiltrout, T. A., Nagashima, K., Back, T. C., and Wiltrout, R. H. (2001). IFN-gamma and Fas/FasL are required for the antitumor and antiangiogenic effects of IL-12/pulse IL-2 therapy. J Clin Invest 108, 51-62.
Xie, L., Pelz, C., Wang, W., Bashar, A., Varlamova, O., Shadle, S., and Impey, S. (2011). KDMSB regulates embryonic stem cell self-renewal and represses cryptic intragenic transcription. The EMBO journal 30, 1473-1484.
Zhu, X., He, F., Zeng, H., Ling, S., Chen, A., Wang, Y., Yan, X., Wei, W., Pang, Y., Cheng, H., et al. (2014). Identification of functional cooperative mutations of SETD2 in human acute leukemia. Nat Genet 46, 287-293.

1. A method for determining suitability of immunotherapy for a subject having cancer, comprising:
analyzing, by RNA analysis, a sample having tumor cells from a subject having cancer to determine whether the tumor cells have a loss of transcriptional fidelity (LTF) phenotype characterized by having a preferential expression or higher proportion of one or more aberrant or non-canonical mRNA isoforms, relative to a control value; and
determining a lack of suitability of immunotherapy where the tumor cells of the subject have an LTF phenotype, or determining a suitability of immunotherapy where the tumor cells of the subject lack an LTF phenotype.

2. The method of claim 1, wherein the control value is that of normal cells, that of non-LTF tumor cells, or that of mRNA corresponding to one or more internal control genes of the tumor cells not affected by LTF.

3. The method of claim 2, wherein the one or more internal control genes of the tumor cells not affected by LTF, comprises one or more type II genes as defined herein.

4. The method of claim 1, wherein the one or more aberrant or non-canonical mRNA isoform(s) comprises aberrant or non-canonical mRNA isoform(s) lacking exon and/or intron sequences found in the corresponding normal or canonical mRNA isoform(s), including full-length isoforms, or retaining exon and/or intron sequences not found in the corresponding normal or canonical mRNA isoform(s), including full-length isoforms.

5. The method of claim 1, wherein the one or more aberrant or non-canonical mRNA isoform(s) comprises aberrant or non-canonical mRNA isoform(s) lacking 5′-exon sequences found in the corresponding normal or canonical mRNA isoform(s), including full-length isoforms, or retaining 5′exon sequences not found in the corresponding normal or canonical mRNA isoform(s), including full-length isoforms.

6. The method of claim 1, wherein the one or more aberrant or non-canonical mRNA isoform(s) comprises aberrant or non-canonical mRNA isoform(s) having an increased amount of retained intron-exon junctions compared to the corresponding normal or canonical mRNA isoform(s), including full-length isoforms.

7. The method of claim 1, wherein the one or more aberrant or non-canonical mRNA isoform(s) comprises an aberrant or non-canonical mRNA lacking exon sequences required for encoding a protein encoded by a corresponding normal or canonical mRNA isoform including full-length mRNA isoforms thereof.

8. The method of claim 7, wherein the aberrant or non-canonical mRNA isoform(s) encode one or more protein(s) that are shorter than the corresponding full-length protein by an amount selected from the group consisting of less than 98%, less than 97%, less than 95%, less than 90%, less than 85%, less than 80%, less than 75%, less than 70%, and less than 60%.

9. The method of claim 1, wherein for a given mRNA, greater than 50%, greater than 60%, greater than 70%, greater than 80%, greater than 90%, or greater than 95% of the mRNA is present as corresponding aberrant or non-canonical mRNA isoforms.

10. The method of claim 1, wherein, for a given mRNA, greater than 50%, greater than 60%, greater than 70%, greater than 80%, greater than 90%, or greater than 95% of the mRNA expression is of the corresponding aberrant or non-canonical mRNA isoform.

11. The method of claim 1, wherein the one or more aberrant or non-canonical mRNA isoforms are aberrant or non-canonical mRNA isoforms of corresponding normal or canonical mRNAs, including full-length mRNAs, having lengths of greater than 10 kb, greater than 25 kb, greater than 40 kb, greater than 50 kb, greater than 75 kb, greater than 100 kb, greater than 150 kb, or greater than 200 kb.

12. The method of claim 1, wherein the one or more aberrant or non-canonical mRNA isoforms are encoded by one or more corresponding genes involved in RNA polymerase II (RNAP II) transcription and/or processing and/or in histone H3 modification and/or chromatin remodeling.

13. The method of claim 12, wherein the RNAP II genes comprise genes involved in RNAP II phosphorylation and/or wherein the genes involved in histone H3 modification and/or chromatin remodeling comprise genes in involved in histone H3 methylation and/or acetylation.

14. The method of claim 13, wherein the genes involved in RNAP II phosphorylation comprise genes involved in RNAP II phosphorylation at amino acid positions Ser2 and/or Ser5.

15. The method of claim 13, wherein the genes involved in histone H3 methylation comprise genes involved in histone H3 methylation at amino acid positions K4, K27, and/or K36.

16. The method of claim 12, wherein the one or more genes involved in RNA polymerase II (RNAP II) transcription and/or processing and/or histone H3 modification and/or chromatin remodeling comprise BAP1, CDK9, CDK7, ASXL2, REST, CCNT1, and/or SETD2.

17. The method of claim 1, wherein the LTF phenotype further comprises reduced expression or reduced presence of one or more proteins selected from the group consisting of RNAP II Ser2, RNAP II Ser5, H3K4me3, H3K27me3, and H3K36me3.

18. The method of claim 17, wherein the sample has reduced expression or reduced presence of at least one of RNAP II Ser2 and/or RNAP II Ser5, and at least one of H3K4me3, and/or H3K27me3, and/or H3K36me3.

19. The method of claim 17, wherein the sample has reduced expression or reduced presence of both RNAP II Ser2 and RNAP II Ser5, and at least one of H3K4me3, and/or H3K27me3, and/or H3K36me3.

20. The method of claim 17, wherein the sample has reduced expression or reduced presence of at least one of RNAP II Ser2 and/or RNAP II Ser5, and at least two of H3K4me3, and/or H3K27me3, and/or H3K36me3.

21. The method of claim 17, wherein the sample has reduced expression or reduced presence of at least one of RNAP II Ser2 and/or RNAP II Ser5, and all three of H3K4me3, and/or H3K27me3, and/or H3K36me3.

22. The method of claim 17, wherein the sample has reduced expression or reduced presence of each of the RNAP II Ser2, RNAP II Ser5, H3K4me3, H3K27me3, and H3K36me3 proteins.

23. The method of claim 17, further comprising overexpression of PEA-15 protein and/or one or more protein synthesis pathway protein(s) and/or reduced expression of one or more proteins selected from the group consisting of NF-κB, EGFR, STAT3, STATS, MAPK, MEK1 (MAP2K1), and derivatives thereof including phosphorylated derivatives thereof including phosphorylated MAPK and phosphorylated NF-κB, and inflammatory response proteins.

24. The method of claim 1, wherein the LTF phenotype further comprises reduced expression of one or more aberrant or non-canonical mRNA isoforms selected from the group consisting of CCNT1, REST, ASXL2, KIF2A, PRKAR1A, NUP84, and NUP100, and/or overexpression of one or more aberrant or non-canonical mRNA isoforms selected from the group consisting of NDUFA3, NDUFA1, PFDN5, PFDN5, DGUOK, and MRPL11.

25. The method of claim 1, wherein the type of cancer comprises one or more selected from the group consisting of cancers of the skin, breast, bladder, kidney, brain, head and neck, pancreas, prostate, liver, lung, ovary, blood, and colon.

26. The method of claim 1, further comprising treating the subject based on the lack of suitability of immunotherapy where the tumor cells of the subject have an LTF phenotype, or based on the suitability of immunotherapy where the tumor cells of the subject lack an LTF phenotype.

27. The method of claim 26, wherein the subject has the LTF phenotype, and wherein the treatment does not comprise immunotherapy, but comprises at least one of chemotherapy and/or targeted therapy and/or alternative therapy, provided that the targeted therapy is not an immunotherapy, or wherein the chemotherapy and/or targeted therapy comprises at least one of sunitinib, everolimus, sirolimus, vemurafenib, and/or trametinib.

28. The method of claim 26, wherein the subject lacks the LTF phenotype, and wherein the treatment comprises immunotherapy.

29. The method of claim 28, wherein the treatment further comprises at least one of chemotherapy and/or targeted therapy and/or alternative therapy, or wherein the chemotherapy and/or targeted therapy comprises at least one of sunitinib, everolimus, sirolimus, vemurafenib, and/or trametinib.

30. The method of claim 28, wherein the immunotherapy comprises administration of one or more interleukin, interferon (IFN), and/or small molecule indoleamine 2,3-dioxygenase (IDO) inhibitor, and/or one or more suitable antibody-based reagent, or one or more checkpoint inhibitory antibodies, including ipilimumab.

31. The method of claim 30, wherein the immunotherapy comprises administration of denileukin diftitox and/or administration of an antibody-based reagent selected from the group consisting of ado-trastuzumab emtansine, alemtuzumab, atezolizumab, bevacizumab, blinatumomab, brentuximab vedotin, cetuximab, catumaxomab, gemtuzumab, ibritumomab tiuxetan, ilipimumab, natalizumab, nimotuzumab, nivolumab, ofatumumab, panitumumab, pembrolizumab, rituximab, tositumomab, trastuzumab, and vivatuxin.

32. The method of claim 26, wherein the treatment is conducted as part of a clinical trial.

33. The method of claim 1, wherein the preferential expression or the higher proportion of the one or more aberrant or non-canonical mRNA isoforms is that of one or more type I genes as defined herein.

34. A method for determining suitability of immunotherapy for a subject having cancer, comprising:
analyzing, by protein analysis, a sample having tumor cells from a subject having cancer to determine whether the tumor cells have a loss of transcriptional fidelity (LTF) phenotype characterized by reduced expression or reduced presence of one or more proteins selected from the group consisting of RNAP II Ser2, RNAP II Ser5, H3K4me3, H3K27me3, and H3K36me3 relative to a respective control value; and
determining a lack of suitability of immunotherapy where the tumor cells of the subject have an LTF phenotype, or determining a suitability of immunotherapy where the tumor cells of the subject lack an LTF phenotype.

35. The method of claim 34, wherein the control value is that of normal cells, or that of non-LTF tumor cells.

36. The method of claim 34, wherein the sample has reduced expression or reduced presence of at least one of RNAP II Ser2 and/or RNAP II Ser5, and at least one of H3K4me3, and/or H3K27me3, and/or H3K36me3.

37. The method of claim 34, wherein the sample has reduced expression or reduced presence of both RNAP II Ser2 and RNAP II Ser5, and at least one of H3K4me3, and/or H3K27me3, and/or H3K36me3.

38. The method of claim 34, wherein the sample has reduced expression or reduced presence of at least one of RNAP II Ser2 and/or RNAP II Ser5, and at least two of H3K4me3, and/or H3K27me3, and/or H3K36me3.

39. The method of claim 34, wherein the sample has reduced expression or reduced presence of at least one of RNAP II Ser2 and/or RNAP II Ser5, and all three of H3K4me3, and/or H3K27me3, and/or H3K36me3.

40. The method of claim 34, wherein the sample has reduced expression or reduced presence of each of the RNAP II Ser2, RNAP II Ser5, H3K4me3, H3K27me3, and H3K36me3.

41. The method of claim 34, wherein the LTF phenotype comprises a preferential expression or higher proportion, relative to that of normal cells, to that of non-LTF tumor cells, or to that of mRNA corresponding to one or more internal control genes of the tumor cells not affected by LTF, of one or more aberrant or non-canonical mRNA isoform(s) of corresponding normal or canonical mRNA isoform(s), including full-length isoforms.

42. The method of claim 41, wherein the one or more aberrant or non-canonical mRNA isoform(s) comprises aberrant or non-canonical mRNA isoform(s) lacking exon sequences required for encoding a protein encoded by a corresponding normal or canonical mRNA isoform, including full-length isoforms.

43. The method of claim 42, wherein the aberrant or non-canonical mRNA isoform(s) encode protein that is is shorter than the corresponding full-length protein by an amount selected from the group consisting of less than 98%, less than 97%, less than 95%, less than 90%, less than 85%, less than 80%, less than 75%, less than 70%, and less than 60%.

44. The method of claim 43, wherein for a given mRNA, greater than 50%, greater than 60%, greater than 70%, greater than 80%, greater than 90%, or greater than 95% of the mRNA is present as corresponding aberrant or non-canonical mRNA isoforms.

45. The method of claim 42, wherein, for a given mRNA, greater than 50%, greater than 60%, greater than 70%, greater than 80%, greater than 90%, or greater than 95% of the mRNA expression is of the corresponding aberrant or non-canonical mRNA isoform.

46. The method of claim 41, wherein the one or more aberrant or non-canonical mRNA isoforms are aberrant or non-canonical mRNA isoforms of corresponding normal or canonical mRNAs, including full-length mRNAs having lengths of greater than 10 kb, greater than 25 kb, greater than 40 kb, greater than 50 kb, greater than 75 kb, greater than 100 kb, greater than 150 kb, or greater than 200 kb.

47. The method of claim 41, wherein the one or more aberrant or non-canonical mRNA isoforms are encoded by one or more corresponding genes involved in RNA polymerase II (RNAP II) transcription and/or processing and/or in histone H3 modification and/or chromatin remodeling.

48. The method of claim 47, wherein the RNAP II genes comprise genes involved in RNAP II phosphorylation and/or wherein the genes involved in histone H3 modification and/or chromatin remodeling comprise genes in involved in histone H3 methylation and/or acetylation.

49. The method of claim 48, wherein the genes involved in RNAP II phosphorylation comprise genes involved in RNAP II phosphorylation at amino acid positions Ser2 and/or Ser5.

50. The method of claim 48, wherein the genes involved in histone H3 methylation comprise genes involved in histone H3 methylation at amino acid positions K4, K27, and/or K36.

51. The method of claim 47, wherein the one or more genes involved in RNA polymerase II (RNAP II) transcription and/or processing and/or histone H3 modification and/or chromatin remodeling comprise BAP1, CDK9, CDK7, ASXL2, REST, CCNT1, and/or SETD2.

52. The method of claim 34, comprising overexpression of PEA-15 protein and/or one or more protein synthesis pathway protein(s) and/or reduced expression of one or more proteins selected from the group consisting of NF-κB, EGFR, STAT3, STATS, MAPK, MEK1 (MAP2K1), and derivatives thereof including phosphorylated derivatives thereof including phosphorylated MAPK and phosphorylated NF-κB, and inflammatory response proteins.

53. The method of claim 34, wherein the LTF phenotype further comprises reduced expression of one or more aberrant or non-canonical mRNA isoforms selected from the group consisting of CCNT1, REST, ASXL2, KIF2A, PRKAR1A, NUP84, and NUP100, and/or overexpression of one or more aberrant or non-canonical mRNA isoforms selected from the group consisting of NDUFA3, NDUFA1, PFDN5, PFDN5, DGUOK, and MRPL11.

54. The method of claim 34, wherein the type of cancer comprises one or more selected from the group consisting of cancers of the skin, breast, bladder, kidney, brain, head and neck, pancreas, prostate, liver, lung, ovary, blood, and colon.

55. The method of claim 34, further comprising treating the subject based on the lack of suitability of immunotherapy where the tumor cells of the subject have an LTF phenotype, or based on the suitability of immunotherapy where the tumor cells of the subject lack an LTF phenotype.

56. The method of claim 55, wherein the subject has the LTF phenotype, and wherein the treatment does not comprise immunotherapy, but comprises at least one of chemotherapy and/or targeted therapy and/or alternative therapy, provided that the targeted therapy is not an immunotherapy, or wherein the chemotherapy and/or targeted therapy comprises at least one of sunitinib, everolimus, sirolimus, vemurafenib, and/or trametinib.

57. The method of claim 55, wherein the subject lacks the LTF phenotype, and wherein the treatment comprises immunotherapy.

58. The method of claim 57, wherein the treatment further comprises at least one of chemotherapy and/or targeted therapy and/or alternative therapy, or wherein the chemotherapy and/or targeted therapy comprises at least one of sunitinib, everolimus, sirolimus, vemurafenib, and/or trametinib.

59. The method of claim 57, wherein the immunotherapy comprises administration of one or more interleukin, interferon (IFN), and/or small molecule indoleamine 2,3-dioxygenase (IDO) inhibitor, and/or one or more suitable antibody-based reagent, including one or more checkpoint inhibitory antibodies including ipilimumab.

60. The method of claim 59, wherein the immunotherapy comprises administration of denileukin diftitox and/or administration of an antibody-based reagent selected from the group consisting of ado-trastuzumab emtansine, alemtuzumab, atezolizumab, bevacizumab, blinatumomab, brentuximab vedotin, cetuximab, catumaxomab, gemtuzumab, ibritumomab tiuxetan, ilipimumab, natalizumab, nimotuzumab, nivolumab, ofatumumab, panitumumab, pembrolizumab, rituximab, tositumomab, trastuzumab, vivatuxin.

61. The method of claim 55, wherein the treatment is conducted as part of a clinical trial.

62. A method of stratifying one or more subjects in a clinical trial, comprising:
analyzing, by RNA and/or protein analysis, a sample having tumor cells from one or more subject(s) having cancer to determine whether the tumor cells have a loss of transcriptional fidelity (LTF) phenotype, wherein the LTF phenotype is characterized by: having a preferential expression or higher proportion of one or more aberrant or non-canonical mRNA isoforms, relative to a control value for expression or proportion; and/or by reduced expression or reduced presence of one or more proteins selected from the group consisting of RNAP II Ser2, RNAP II Ser5, H3K4me3, H3K27me3, and H3K36me3 relative to a respective control value of expression or presence of RNAP II Ser2, RNAP II Ser5, H3K4me3, H3K27me3, and H3K36me3; and
determining a lack of suitability of immunotherapy where the tumor cells of the subject have an LTF phenotype, or determining a suitability of immunotherapy where the tumor cells of the subject lack an LTF phenotype.

63. The method of claim 62, wherein the control value for expression or proportion is that of normal cells, that of non-LTF tumor cells, or that of mRNA corresponding to one or more internal control genes of the tumor cells not affected by LTF.

64. The method of claim 63, wherein the one or more internal control genes of the tumor cells not affected by LTF, comprises one or more type II genes as defined herein.

65. The method of claim 62, wherein the control value of expression or presence of RNAP II Ser2, RNAP II Ser5, H3K4me3, H3K27me3, and H3K36me3 is that of normal cells, or that of non-LTF tumor cells.

66. The method of claim 62, further comprising treating the subject based on the lack of suitability of immunotherapy where the tumor cells of the subject have an LTF phenotype, or based on the suitability of immunotherapy where the tumor cells of the subject lack an LTF phenotype.

67. A diagnostic kit, test, or array to test for presence of a loss of transcriptional fidelity (LTF) phenotype in a sample, comprising:
materials for quantification of phosphorylation at amino acid position RNAP II Ser2, and/or RNAP II Ser5; and/or
materials for methylation analysis at amino acid position H3K4me3, H3K27me3, and H3K36me3 proteins; and/or
materials for determining the presence or absence of transcriptional fidelity (LTF) phenotype characterized by having a preferential expression or higher proportion, relative to normal cells or to non-LTF tumor cells, of one or more aberrant or non-canonical mRNA isoform(s), relative to a control value.

68. The kit of claim 67, wherein the control value is that of normal cells, that of non-LTF tumor cells, or that of mRNA corresponding to one or more internal control genes of the tumor cells not affected by LTF.

69. The kit of claim 68, wherein the one or more internal control genes of the tumor cells not affected by LTF, comprises one or more type II genes as defined herein.
